(19) United States
(12) Patent Application Publication
(10) Pub. No.: US 2014/0066410 A1
(43) Pub. Date: Mar. 6, 2014

(54) INHIBITORS OF BROMODOMAINS AS MODULATORS OF GENE EXPRESSION

(71) Applicants: Ming-Ming Zhou, Old Greenwich, CT (US); Michael Ohlmeyer, Plainsboro, NJ (US); Shiraz Mujtaba, Flushing, NY (US); Alexander Plotnikov, Brooklyn, NY (US); David Kastrinsky, Fair Lawn, NJ (US); Guangtao Zhang, New York, NY (US)

(72) Inventors: Ming-Ming Zhou, Old Greenwich, CT (US); Michael Ohlmeyer, Plainsboro, NJ (US); Shiraz Mujtaba, Flushing, NY (US); Alexander Plotnikov, Brooklyn, NY (US); David Kastrinsky, Fair Lawn, NJ (US); Guangtao Zhang, New York, NY (US)

(73) Assignee: ICAHN SCHOOL OF MEDICINE AT MOUNT SINAI, New York, NY (US)

(21) Appl. No.: 14/001,074

(22) PCT Filed: Feb. 23, 2012
(86) PCT No.: PCT/US12/26308

§ 371 Date: Nov. 18, 2013

Related U.S. Application Data
(60) Provisional application No. 61/445,859, filed on Feb. 23, 2011.

(51) Int. Cl. C07D 213/76; C07C 311/44

(52) U.S. Cl. CPC C07D 213/76; C07C 311/44
USPC 514/150; 546/312; 514/352; 534/730; 534/731

(57) ABSTRACT

This disclosure relates generally to compounds and compositions comprising one or more diphenylethylene, diphenylethylyne, and azobenzene analogs. These compounds are useful for treating diseases associated with NF-kB and p53 activity, such as cancer and inflammatory disease.

### CROSS-REFERENCE TO RELATED APPLICATIONS

This application claims priority to U.S. Application Ser. No. 61/445,859, filed on Feb. 23, 2011, which is incorporated by reference in its entirety herein.

### FEDERALLY SPONSORED RESEARCH OR DEVELOPMENT

The U.S. Government has certain rights in this invention pursuant to Grant No. R01HG004508-03 awarded by the National Institutes of Health/National Human Genome Research Institute.

### TECHNICAL FIELD

This disclosure relates generally to compounds and compositions comprising one or more diphenylethylene, diphenylethylyne, and azobenzene analogs. These compounds are useful for treating diseases associated with NF-kB and p53 activity, such as cancer and inflammatory diseases.

### BACKGROUND

Cardiovascular diseases continue to be an epidemic in the United States and the Western world. The salient feature of cardiac ischemia, which is mainly due to coronary syndromes, includes lack of oxygen and nutrition, which generates stress signals to activate pathways leading to cardiac myocyte death. It has been reported that ischemia-induced myocyte DNA damage results in enhanced transcriptional activity of the tumor suppressor p53 as well as p53-dependent cardiac myocyte apoptosis; the latter is a key feature in the progression of ischemic heart disease. Myocardial ischemia can also induce inflammatory responses and cardiomyocyte necrosis, depending on the intensity and duration of ischemia and reperfusion. Previous studies have shown that exposure of myocytes to hypoxia results in increased p53 trans-activating activity and protein accumulation along with the expression of p21/WAF-1/CIP-1, a well-characterized target of p53 transactivation. While p53 activation has been recognized for therapeutic potential in cancer treatments, its hyper-activation could also be detrimental in both normal and ischemic conditions. Therefore, in a different biological context, modulation of p53 function as a transcriptional regulator, either activation or inhibition, could present valid therapeutic opportunities.

### SUMMARY

As a transcription factor in cellular responses to external stress, tumor suppressor p53 is tightly regulated. Excessive p53 activity during myocardial ischemia can cause irreversible cellular injury and cardiomyocyte death. p53 activation is dependent on lysine acetylation by the lysine acetyltransferase and transcriptional co-activator CBP (CREB-binding protein) and on acetylation-directed CBP recruitment for p53 target gene expression. Provided herein are inhibitors (e.g., compounds of formula (1) and (2)) of the acetyl-lysine binding activity of the bromodomain of CBP. In some embodiments, a compound provided herein can alter post-translational modifications on p53 and histones, inhibit p53 interaction with CBP and transcriptional activity in cells, and prevent apoptosis in ischemic cardiomyocytes. In addition, the compounds provided herein provide are useful in the treatment of human disorders such as myocardial ischemia, cancer, and inflammatory diseases.

Provided herein is a compound of formula (1):

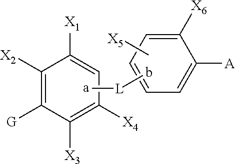

or a pharmaceutically acceptable salt form thereof, wherein:
A is selected from the group consisting of:

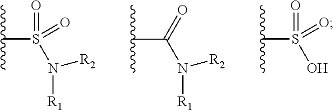

L is a linking group selected from:

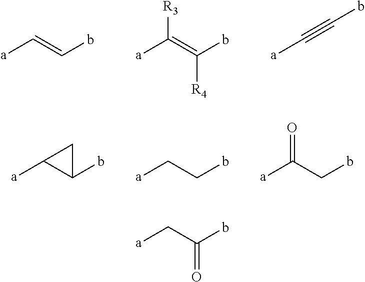

G is a heteroatom containing group capable of accepting a hydrogen bond or donating a hydrogen bond, or G is fused to X₂ or X₃ to form a heterocyclic ring system capable of accepting or donating a hydrogen bond;
X₁, and X₄ are independently selected from the group consisting of: H, C₁₋₁₀ alkyl, C₁₋₁₀ perfluoroalkyl, halogen, nitrile, hydroxy, C₁₋₁₀ alkoxy, C₁₋₁₀ perfluoroalkoxy, C₁₋₁₀ thioalkyl, C₁₋₁₀ perfluoroalkyl, amine, alkylamino, C₁₋₁₀ acylamino, aryl, heteroaryl, carboxamido, carboxyl, and carboalkoxy;
X₂ and X₃ are independently selected from the group consisting of: H, C₁₋₁₀ alkyl, C₁₋₁₀ perfluoroalkyl, halogen, nitrile, hydroxy, C₁₋₁₀ alkoxy, C₁₋₁₀ perfluoroalkoxy, C₁₋₁₀ thioalkyl, C₁₋₁₀ perfluoroalkyl, amine, alkylamino, C₁₋₁₀ acylamino, aryl, heteroaryl, carboxamide, and C₂₋₁₀ acyl;
optionally, X₁ and X₂ may come together to form a cycloalkyl, heterocycloalkyl, aromatic or heteroaromatic ring system;
X₅ and X₆ are independently selected from the group consisting of: H, C₁₋₁₀ alkyl, C₁₋₁₀ alkoxy, C₁₋₁₀ perfluoroalkyl, halogen, and nitrile;
R₁ is selected from the group consisting of: substituted or unsubstituted aryl, substituted or unsubstituted heteroaryl, and substituted or unsubstituted C₁₋₁₀ alkyl;
R₂ is selected from the group consisting of: H and C₁₋₁₀ alkyl;
optionally, R₁ and R₂ may come together to form a substituted or unsubstituted heterocycloalkyl ring system; and
R₃ and R₄ are independently selected from the group consisting of: H and C₁₋₁₀ alkyl.

In some embodiments, A is:

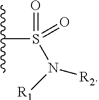

In some embodiments, L is selected from the group consisting of:

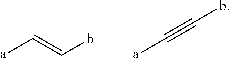

In some embodiments, G is fused to X₂ or X₃ to form a heterocyclic ring system capable of accepting or donating a hydrogen bond. For example, the heterocyclic ring system can be selected from the group consisting of: azetidinyl, pyrrolyl, imidazolyl, pyrazolyl, pyridinyl, pyrazinyl, pyrimidinyl, pyridazinyl, indolizinyl, isoindolyl, indolyl, dihydroindolyl, indazolyl, furanyl, purinyl, quinolizinyl, isoquinolinyl, quinolinyl, phthalazinyl, naphthylpyridinyl, quinoxalinyl, quinazolinyl, cinnolinyl, pteridinyl, carbazolyl, carbolinyl, phenanthridinyl, acridinyl, phenanthrolinyl, isothiazolyl, phenazinyl, isoxazolyl, phenoxazinyl, phenothiazinyl, imidazolidinyl, imidazolinyl, imidazolyl, piperidinyl, piperazinyl, indolinyl, phthalimidyl, 1,2,3,4-tetrahydroisoquinolinyl, 4,5,6,7-tetrahydrobenzo[b]thiophenyl, thiazolyl, thiazolidinyl, thiophenyl, benzo[b]thiophenyl, morpholino, thiomorpholino, piperidinyl, pyrrolidinyl, and tetrahydrofuranyl. In some embodiments, the heterocyclic ring system is selected from imidazolyl, and pyrrolyl.

In some embodiments, G is selected from the group consisting of: OH, CH₂OH, NH₂, SH, C(O)H, CO₂H, OC(O)HCN, NHC(O)H, NH(SO₂)H, NHC(O)NH₂, NHCN, CH(CN)₂, F, Cl, OSO₃H, ONO₂H, and NO₂. For example, G can be selected from OH and OH bioisosteres. In some embodiments, G is OH.

In some embodiments, X₁ is selected from the group consisting of: H and amine. For example, X₁ can be an amine, such as a protected amine. In some embodiments, the protected amine is selected from the group consisting of: acylamine and alkoxycarbonylamine.

In some embodiments, X₂ is selected from H and C₁₋₁₀ alkyl. For example, X₂ can be CH₃.

In some embodiments, X₃ is selected from H and C₁₋₁₀ alkyl. For example, X₃ is CH₃.

In some embodiments, X₄ is H. In some embodiments, X₅ and X₆ are H.

In some embodiments, R₁ is a substituted aryl. For example, the substituted aryl can be a naphyl or anthracyl moiety. In some embodiments, R₁ is a substituted or unsubstituted heteroaryl. For example, the substituted heteroaryl can be a quinolyl moiety. In some embodiments, R₁ the unsubstituted heteroaryl is pyridinyl.

In some embodiments, R₁ and R₂ come together to form a substituted or unsubstituted heterocycloalkyl ring system. For example, the heterocycloalkyl ring system can be selected from piperidinyl, morpholino, and tetrahydroquinolinyl.

In some embodiments, R₁ is H.

In some embodiments, the compound is a compound of formula (1A):

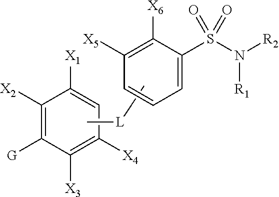

or a pharmaceutically acceptable salt form thereof, wherein:
L is selected from the group consisting of:

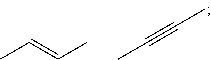

G is selected from the group consisting of: OH, CH₂OH, NH₂, SH, C(O)H, CO₂H, OC(O)HCN, NHC(O)H, NH(SO₂)H, NHC(O)NH₂, NHCN, CH(CN)₂, F, Cl, OSO₃H, ONO₂H, and NO₂, or G is fused to X₂ to form a heterocyclic ring system capable of accepting or donating a hydrogen bond;
X₁ is a protected or unprotected amine;
X₂ and X₃ are independently selected from the group consisting of: H, C₁₋₁₀ alkyl, halogen;
X₄, X₅, and X₆ are H;
R₁ is selected the group consisting of: substituted C₁₋₁₀ alkyl, aryl, and heteroaryl;
R₂ is H.

In some embodiments, G is OH. In some embodiments, X₁ is a protected amine. For example, the protected amine can be selected from the group consisting of: acylamine and alkoxycarbonylamine. In some embodiments, X₂ is selected from H and C₁₋₁₀ alkyl. For example, X₂ can be CH₃. In some embodiments, X₃ is selected from H and C₁₋₁₀ alkyl. For example, X₃ can be CH₃. In some embodiments, R₁ is a heteroaryl. For example, the unsubstituted heteroaryl can be pyridinyl.

Non-limiting examples of a compound of formula (1) includes:

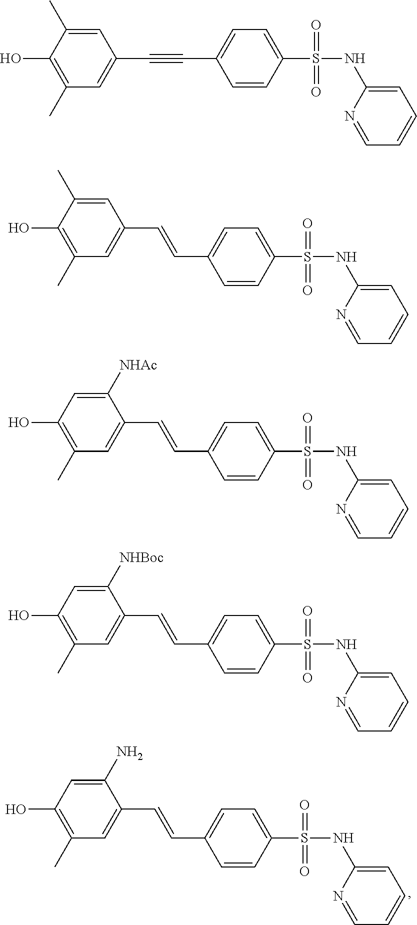 [SMILES: Cc1cc(C#Cc2ccc(S(=O)(=O)Nc3ccccn3)cc2)cc(C)c1O ; Cc1cc(/C=C/c2ccc(S(=O)(=O)Nc3ccccn3)cc2)cc(C)c1O ; CC(=O)Nc1cc(O)c(C)cc1/C=C/c1ccc(S(=O)(=O)Nc2ccccn2)cc1 ; Cc1cc(/C=C/c2ccc(S(=O)(=O)Nc3ccccn3)cc2)c(NC(=O)OC(C)(C)C)cc1O ; Cc1cc(/C=C/c2ccc(S(=O)(=O)Nc3ccccn3)cc2)c(N)cc1O]

or a pharmaceutically acceptable salt thereof.

Also provided herein is a compound of formula (2):

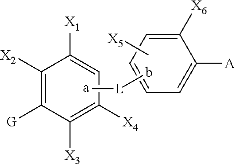

or a pharmaceutically acceptable salt form thereof, wherein:
A is selected from the group consisting of:

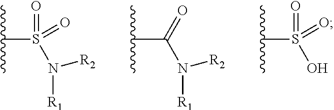

L is:

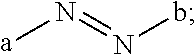

G is a heteroatom containing group capable of accepting a hydrogen bond or donating a hydrogen bond, or G is fused to X₂ or X₁ to form a heterocyclic ring system capable of accepting or donating a hydrogen bond;
X₁ and X₄ are independently selected from the group consisting of: H, C₁₋₁₀ alkyl, C₁₋₁₀ perfluoroalkyl, halogen, nitrile, hydroxy, C₁₋₁₀ alkoxy, C₁₋₁₀ perfluoroalkoxy, C₁₋₁₀ thioalkyl, C₁₋₁₀ perfluoroalkyl, amine, alkylamino, C₁₋₁₀ acylamino, aryl, heteroaryl, carboxamido, carboxyl, and carboalkoxy;
X₂ and X₃ are independently selected from the group consisting of: H, C₁₋₁₀ alkyl, C₁₋₁₀ perfluoroalkyl, halogen, nitrite, hydroxy, C₁₋₁₀ alkoxy, C₁₋₁₀ perfluoroalkoxy, C₁₋₁₀ thioalkyl, C₁₋₁₀ perfluoroalkyl, amine, alkylamino, C₁₋₁₀ acylamino, aryl, heteroaryl, carboxamide, and C₂₋₁₀ acyl;
optionally, X₁ and X₂ may come together to form a cycloalkyl, heterocycloalkyl, aromatic or heteroaromatic ring system;
X₅ and X₆ are independently selected from the group consisting of: H, C₁₋₁₀ alkyl, C₁₋₁₀ alkoxy, C₁₋₁₀ perfluoroalkyl, halogen, and nitrile;
R₁ is selected from the group consisting of: substituted or unsubstituted aryl, substituted or unsubstituted heteroaryl, and substituted or unsubstituted C₁₋₁₀ alkyl;
R₂ is selected from the group consisting of: H and C₁₋₁₀ alkyl;
optionally, R₁ and R₂ may come together to form a substituted or unsubstituted heterocycloalkyl ring system; and
R₃ and R₄ are independently selected from the group consisting of: H and C₁₋₁₀ alkyl.

In some embodiments, A is:

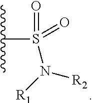

In some embodiments, G is fused to X₂ or X₃ to form a heterocyclic ring system capable of accepting or donating a hydrogen bond. For example, G can be selected from the group consisting of: azetidinyl, pyrrolyl, imidazolyl, pyrazolyl, pyridinyl, pyrazinyl, pyrimidinyl, pyridazinyl, indolizinyl, isoindolyl, indolyl, dihydroindolyl, indazolyl, furanyl, purinyl, quinolizinyl, isoquinolinyl, quinolinyl, phthalazinyl, naphthylpyridinyl, quinoxalinyl, quinazolinyl, cinnolinyl, pteridinyl, carbazolyl, carbolinyl, phenanthridinyl, acridinyl, phenanthrolinyl, isothiazolyl, phenazinyl, isoxazolyl, phenoxazinyl, phenothiazinyl, imidazolidinyl, imidazolinyl, imidazolyl, piperidinyl, piperazinyl, indolinyl, phthalimidyl, 1,2,3,4-tetrahydroisoquinolinyl, 4,5,6,7-tetrahydrobenzo[b]thiophenyl, thiazolyl, thiazolidinyl, thiophenyl, benzo[b]thiophenyl, morpholino, thiomorpholino, piperidinyl, pyrrolidinyl, and tetrahydrofuranyl. In some embodiments, the heterocyclic ring system is selected from imidazolyl, and pyrrolyl. In some embodiments, G is selected from OH and OH bioisosteres. For example, G can be OH.

In some embodiments, X₁ is selected from the group consisting of: H, C₁₋₁₀ alkyl, and amine. For example, X₁ can be H.

In some embodiments, X₂ and X₃ are independently selected from the group consisting of: H, halogen, C₁₋₁₀ alkyl, C₁₋₁₀ perfluoroalkyl, and C₁₋₁₀ alkoxy.

In some embodiments, X₄ is H. In some embodiments, X₅ and X₆ are H.

In some embodiments, R₁ is a substituted aryl. For example, the substituted aryl is a naphyl or anthracyl moiety. In some embodiments, R₁ is a substituted or unsubstituted heteroaryl. For example, the heteroaryl can be selected from quinolyl and pyridinyl. In some embodiments, R₁ and R₂ come together to form a substituted or unsubstituted heterocycloalkyl ring system. For example, the heterocycloalkyl ring system is selected from piperidinyl, morpholino, and tetrahydroquinolinyl. In some embodiments, R₂ is H.

In some embodiments, the compound is a compound of formula (2A):

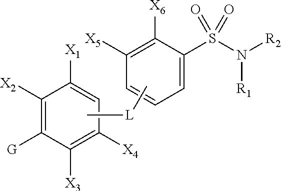

or a pharmaceutically acceptable salt form thereof, wherein:
L is:

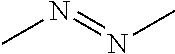

G is selected from the group consisting of: OH, CH₂OH, NH₂, SH, C(O)H, CO₂H, OC(O)HCN, NHC(O)H, NH(SO₂)H, NHC(O)NH₂, NHCN, CH(CN)₂, F, Cl, OSO₃H, ONO₂H, and NO₂;
X₁ is H or a protected or unprotected amine;
X₂ and X₃ are independently selected from the group consisting of: H, halogen, hydroxyl, Cl₁₋₁₀ alkyl, C₁₋₁₀ perfluoroalkyl, and C₁₋₁₀ alkoxy;
X₄ is H;
X₅ and X₆ are independently selected from the group consisting of: H, halogen, hydroxyl, C₁₋₁₀ alkyl, and C₁₋₁₀ alkoxy;
R₁ is selected the group consisting of: substituted C₁₋₁₀ alkyl, aryl, and heteroaryl; and
R₂ is H.

In some embodiments, G is OH. In some embodiments, X₁ is an unprotected amine. In some embodiments, X₂ is selected from H and C₁₋₁₀ alkyl. In some embodiments, X₃ is selected from H and C₁₋₁₀ alkyl. In some embodiments, R₁ is a heteroaryl. For example, the heteroaryl can be a pyridinyl.

In some embodiments, the compound is a compound of formula (2B):

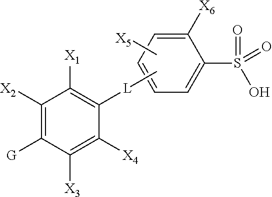

or a pharmaceutically acceptable salt form thereof, wherein:
L is:

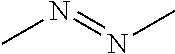

G is OH;
X₁ and X₄ are H;
X₂ and X₃ are independently selected from the group consisting of: H, halogen, hydroxyl, C₁₋₁₀ alkyl, C₁₋₁₀ perfluoroalkyl, and C₁₋₁₀ alkoxy; and
X₅ and X₆ are independently selected from the group consisting of: H, halogen, hydroxyl, C₁₋₁₀ alkyl, and C₁₋₁₀ alkoxy.

Non-limiting examples of a compound of formula (2) include:

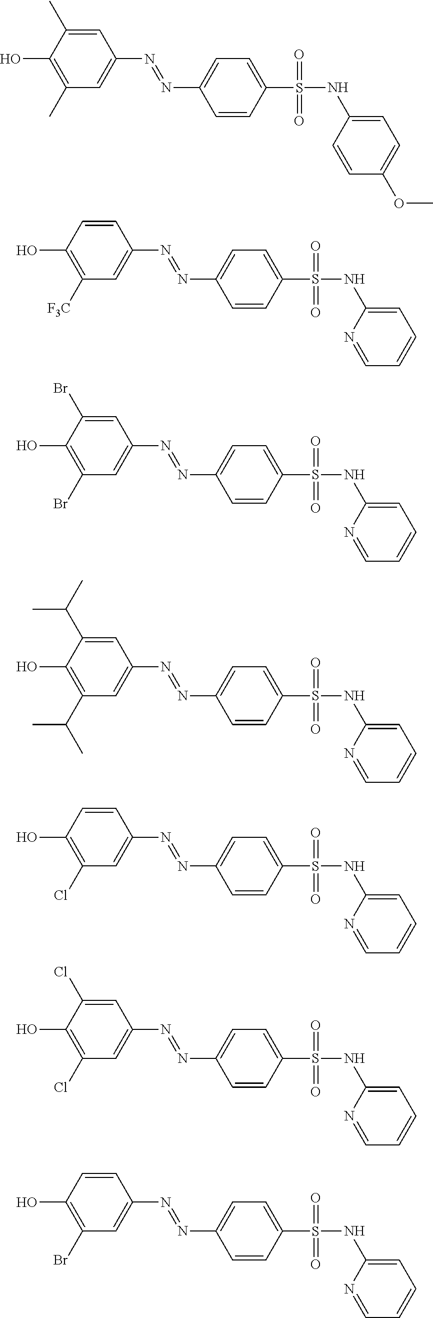

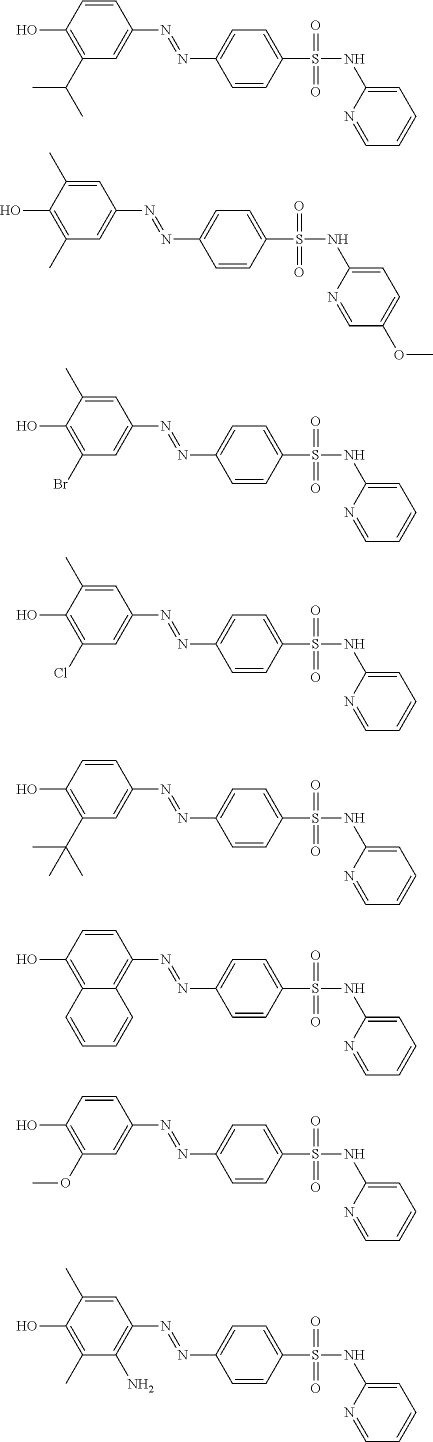

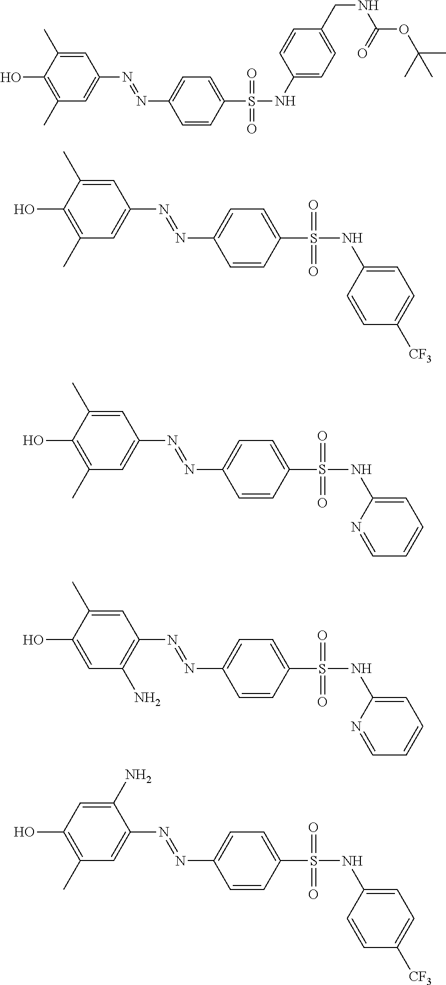

or a pharmaceutically acceptable salt form thereof.

Further provided herein are pharmaceutical compositions comprising a compound of formula (1) or (2), or a pharmaceutically acceptable salt thereof, and a pharmaceutically acceptable excipient.

The compounds provided herein are useful in a number of therapeutic methods. For example, provided herein is a method of treating cancer in a patient, the method comprising administering a therapeutically effective amount of a compound of formula (1) or (2), or a pharmaceutically acceptable salt form thereof, to the patient. In some embodiments, the cancer is selected from the group consisting of: B cell lymphoma, Hodgkins disease, T cell lymphoma, adult T cell lymphoma, adult T cell leukemia, acute lymphoblastic leukemia, breast cancer, liver cancer, thyroid cancer, pancreatic cancer, prostate cancer, melanoma, head and neck SCC, colon cancer, multiple myeloma, ovarian cancer, bladder cancer, and lung carcinoma. In some embodiments, the method further comprises administering a therapeutically effective amount of an anticancer agent to the patient. For example, the anticancer agent can be selected from the group consisting of: irinotecan, daunorubicin, doxorubicin, vinblastine, vincristine, etoposide, actinmycin D, cisplatin, paclitaxel, gemcitabine, SAHA, and combinations thereof. In some embodiments, the patient is resistant to one or more cytotoxic chemotherapeutic agents.

Also provided herein is a method for modulating gene transcription in a patient by inhibiting recruitment of bromodomain containing transcriptional co-activators, transcription regulator proteins, or chromatin remodeling regulator proteins to chromatin, the method comprising administering a therapeutically effective amount of a compound of formula (1) or (2), or a pharmaceutically acceptable salt form thereof, to the patient.

A method for modulating gene transcription in a patient by inhibiting lysine acetylation of histones, transcription regulator proteins, transcriptional co-activators, or other chromatin-associated proteins by bromodomain containing histone acetyltransferase (HAT) transcriptional co-activators is provided herein, the method comprising administering a therapeutically effective amount of a compound of formula (1) or (2), or a pharmaceutically acceptable salt form thereof, to the patient.

Further provided herein is a method for modulating gene transcription in a patient by inhibiting interactions between bromodomain containing transcriptional co-activators, transcription regulator proteins, chromatin remodeling regulator proteins, and other chromatin-associated proteins in complexes that are required for gene transcription, the method comprising administering a therapeutically effective amount of a compound of formula (1) or (2), or a pharmaceutically acceptable salt form thereof, to the patient.

In the methods described above, the transcriptional co-activator, transcription regulator protein, or chromatin remodeling regulator protein can be selected from the group selected from: PCAF, GCN5L2, p300/CBP, TAF1, TAF1L, Ash1L, MLLx, SMARCA2, SMARCA4, BRPF1, ATAD2, BRD7, BRD2, BRD3, BRD4, BRDT, BAZ1B (WSTF), BAZ2B, BPTF, SP140L, TRIM24, TRIM33, or a combination thereof. In some embodiments, the methods can further comprise administrating a therapeutically effective amount of a histone acetyltransferase inhibitor to the patient.

Also provided herein is a method for modulating the transcriptional activity of PCAF in HIV transcriptional activity and replication in a patient, the method comprising administering a therapeutically effective amount of a compound of formula (1) or (2), or a pharmaceutically acceptable salt form thereof, to the patient. For example, a method for treating HIV/AIDS in a patient is provided, the method comprising administering a therapeutically effective amount of a compound of formula (1) or (2), or a pharmaceutically acceptable salt form thereof, to the patient. In some embodiments, PCAF transcriptional activity in the patient is modulated.

Further provided herein is a method for modulating the transcriptional activity of NF-kB and its target genes in a patient, the method comprising, administering a therapeutically effective amount of a compound of formula (1) or (2), or a pharmaceutically acceptable salt form thereof, to the patient.

This disclosure also provides a method of treating a disease where NF-kB is over-activated in a patient, the method comprising administering a therapeutically effective amount of a compound of formula (1) or (2), or a pharmaceutically acceptable salt form thereof, to the patient. In some embodiments, the disease is cancer. For example, the cancer can be selected from the group consisting of: B cell lymphoma, Hodgkins disease, T cell lymphoma, adult T cell lymphoma, adult T cell leukemia, acute lymphoblastic leukemia, breast cancer, liver cancer, thyroid cancer, pancreatic cancer, prostate cancer, melanoma, head and neck SCC, colon cancer, multiple myeloma, ovarian cancer, bladder cancer, and lung carcinoma.

Also provided herein is a method of inducing stem cell differentiation in a patient, the method comprising administering a therapeutically effective amount of a compound of claim 1 or 39, or a pharmaceutically acceptable salt form thereof, to the patient. For example, the stem cells can be cancer stem cells. In some embodiments, the method further comprises administrating a therapeutically effective amount of a histone acetyltransferase inhibitor to the patient.

Further provided herein is a method of inducing apoptosis of malignant cells in a patient, the method comprising administering a therapeutically effective amount of a compound of formula (1) or (2), or a pharmaceutically acceptable salt form thereof, to the patient.

This disclosure provides a method of treating an inflammatory disease or autoimmune disease in a patient, the method comprising administering a therapeutically effective amount of a compound of formula (1) or (2), or a pharmaceutically acceptable salt form thereof, to the patient. In some embodiments, NF-kB is implicated in the pathology of the disease. In some embodiments, the inflammatory disease or autoimmune disease is selected from the group consisting of: rheumatoid arthritis (RA), inflammatory bowel disease (IBD), multiple sclerosis (MS), type 1 diabetes, lupus, asthma, psoriasis, and post ischemic inflammation. For example, the post ischemic inflammation can be selected from stroke and myocardial infarction.

Also provided herein is a method of treating a neurological disorder in a patient where NF-kB is implicated in the pathology of the disorder, the method comprising administering a therapeutically effective amount of a compound of claim 1 or 39, or a pharmaceutically acceptable salt form thereof, to the patient. In some embodiments, the neurological disorder is selected from Alzheimer's disease and Parkinson's disease.

Further provided herein is a method of treating a metabolic disease in a patient where NF-kB is implicated in the pathology of the disease, the method comprising administering a therapeutically effective amount of a compound of formula (1) or (2), or a pharmaceutically acceptable salt form thereof, to the patient. In some embodiments, the metabolic disease is type 2 diabetes mellitus.

This disclosure also provides a method for regulating P-TEFb in a patient, the method comprising administering a therapeutically effective amount of a compound of claim 1 or 39, or a pharmaceutically acceptable salt form thereof, to the patient. In some embodiments, P-TEFb is regulated by binding the bromodomains of BRD4.

Also provided herein is a method for treating a retroviral infection in a patient, the method comprising administering a therapeutically effective amount of a compound of formula (1) or (2), or a pharmaceutically acceptable salt form thereof, to the patient.

Further provided herein is a method for treating myocardial hypertrophy in a patient, the method comprising administering a therapeutically effective amount of a compound of formula (1) or (2), or a pharmaceutically acceptable salt form thereof, to the patient.

This disclosure provides a method for modulating the transcriptional activity of human p53 and activation of its target genes in a patient, the method comprising administering a therapeutically effective amount of a compound of claim 1 or 39, or a pharmaceutically acceptable salt form thereof, to the patient. In some embodiments, the modulating is down-regulating. For example, the down-regulating of p53 transcription activity enhances the reprogramming efficiency of induced pluripotent stem cells using one or more stem cell factors selected from Oct3/4, Sox2, Klf4, and c-Myc. In some embodiments, the modulating is useful in the treatment of disease or condition wherein p53 activity is hyper-activated under a stress-induced event. For example, the stress-induced event is selected from the group selected from: trauma, hyperthermia, hypoxia, ischemia, stroke, a burn, a seizure, a tissue or organ prior to transplantation, and a chemo- or radiation therapy treatment.

Further provided herein is a method for modulating the transcriptional activity of transcription co-activators CBP/p300 by binding to the bromodomain in a patient, the method comprising administering a therapeutically effective amount of a compound of formula (1) or (2), or a pharmaceutically acceptable salt form thereof, to the patient. In some embodiments, CBP/p300 activity is associated with inducing or promoting a disease or condition selected from the group consisting of: cancer, acute myeloid leukemia (AML), chronic myeloid leukemia, circadian rhythm disorders, and drug addiction.

This disclosure provides a method for modulating the transcriptional activity of Williams-Beuren syndrome transcription factor (WSTF) by binding to the bromodomain in a patient, the method comprising administering a therapeutically effective amount of a compound of formula (1) or (2), or a pharmaceutically acceptable salt form thereof, to the patient. In some embodiments, the WSTF hyper-activity modulated occurs in an over-expressed vitamin A receptor complex in one or more of a cancer of the breast, head and neck, and lungs, leukemia, and skin cancers.

Also provided herein is a method for modulating gene transcription in a cell by inhibiting recruitment of bromodomain containing transcriptional co-activators, transcription regulator proteins, or chromatin remodeling regulator proteins to chromatin, the method comprising contacting the cell with a therapeutically effective amount of a compound of formula (1) or (2), or a pharmaceutically acceptable salt form thereof.

Further provided herein is a method for modulating gene transcription in a cell by inhibiting lysine acetylation of histones, transcription regulator proteins, transcriptional co-activators, or other chromatin-associated proteins by bromodomain containing histone acetyltransferase (HAT) transcriptional co-activators, the method comprising contacting the cell with a therapeutically effective amount of a compound of formula (1) or (2), or a pharmaceutically acceptable salt form thereof.

This disclosure also provides a method for modulating gene transcription in a cell by inhibiting interactions between bromodomain containing transcriptional co-activators, transcription regulator proteins, chromatin remodeling regulator proteins, and other chromatin-associated proteins in complexes that are required for gene transcription, the method comprising contacting the cell with a therapeutically effective amount of a compound of formula (1) or (2), or a pharmaceutically acceptable salt form thereof. In some embodiments, the transcriptional co-activator, transcription regulator protein, or chromatin remodeling regulator protein is selected from the group selected from: PCAF, GCN5L2, p300/CBP, TAF1, TAF1L, Ash1L, MLL, SMARCA2, SMARCA4, BRPF1, ATAD2, BRD7, BRD2, BRD3, BRD4, BRDT, BAZ1B (WSTF), BAZ2B, BPTF, SP140L, TRIM24, TRIM33, or a combination thereof.

In the methods described above, the method can further comprise contacting the cell with a therapeutically effective amount of a histone acetyltransferase inhibitor.

Also provided herein is a method for modulating the transcriptional activity of PCAF in HIV transcriptional activity and replication in a cell, the method comprising contacting the cell with a therapeutically effective amount of a compound of formula (1) or (2), or a pharmaceutically acceptable salt form thereof.

Further provided herein is a method for modulating the transcriptional activity of NF-kB and its target genes in a cell, the method comprising contacting the cell with a therapeutically effective amount of a compound of formula (1) or (2), or a pharmaceutically acceptable salt form thereof.

This disclosure also provides a method of inducing stem cell differentiation in a cell, the method comprising contacting the cell with a therapeutically effective amount of a compound of formula (1) or (2), or a pharmaceutically acceptable salt form thereof. In some embodiments, the stem cells are cancer stem cells. In some embodiments, the method further comprises contacting the cell with a therapeutically effective amount of a histone acetyltransferase inhibitor.

Also provided herein is a method of inducing apoptosis of a malignant cell, the method comprising contacting the cell with a therapeutically effective amount of a compound of formula (1) or (2), or a pharmaceutically acceptable salt form thereof.

Further provided herein is a method for regulating P-TEFb in a cell, the method comprising contacting the cell with a therapeutically effective amount of a compound of formula (1) or (2), or a pharmaceutically acceptable salt form thereof. In some embodiments, P-TEFb is regulated by binding the bromodomains of BRD4.

This disclosure also provides a method for modulating the transcriptional activity of human p53 and activation of its target genes in a cell, the method comprising contacting the cell with a therapeutically effective amount of a compound of formula (1) or (2), or a pharmaceutically acceptable salt form thereof. In some embodiments, the modulating is down-regulating. For example, the down-regulating of p53 transcription activity enhances the reprogramming efficiency of induced pluripotent stem cells using one or more stem cell factors selected from Oct3/4, Sox2, Klf4, and c-Myc.

Also provided herein is a method for modulating the transcriptional activity of transcription co-activators CBP/p300 by binding to the bromodomain in a cell, the method comprising contacting the cell with a therapeutically effective amount of a compound of formula (1) or (2), or a pharmaceutically acceptable salt form thereof.

Further provided herein is a method for modulating the transcriptional activity of Williams-Beuren syndrome transcription factor (WSTF) by binding to the bromodomain in a cell, the method comprising contacting the cell with a therapeutically effective amount of a compound of formula (1) or (2), or a pharmaceutically acceptable salt form thereof, to the patient.

This disclosure also provides a method of treating disease or disorder with a compound that blocks the acetyl-lysine binding activity of a bromodomain containing transcriptional co-activator, transcription regulator protein or chromatin remodeling regulator protein, leading to attenuated gene transcriptional activity that induces or contributes to said disease or disorder. In some embodiments, the compound makes hydrogen bond contacts with an acetyl-lysine binding asparagine residue of a bromodomain containing transcriptional co-activator, transcription regulator protein, or chromatin remodeling regulator protein, leading to attenuated transcriptional activity that induces or contributes to said disease or disorder.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF THE DRAWINGS

FIG. 1. Functional characterization of CBP BRD chemical modulators in transcription. (A) Dose-dependent inhibition of p21 luciferase activity in U20S cells upon treatment of ischemin or MS119. The luciferase activity was normalized to renilla luciferase as a control. The IC₅₀ was calculated using PRISM software. (B) Effects of the CBP BRD ligands on BRDU incorporation in U20S cells upon doxorubicin treatment. The data showing that ischemin or MS119 prevents a doxorubicin-induced decrease of BRDU incorporation.

FIG. 2. Effects of ischemin on p53 activation induced by DNA damage. (A) Immunoblots showing ischemin effects on levels of endogenous p53, p53 phosphorylation on serine 15, p53 acetylation on lysine 382, as well as p53 target genes. (B) Immunoblots showing ischemin effects on levels of correlated H3K9 acetylation and H3S10 phosphorylation, and unaffected upstream kinases CHK1 and ATM upon doxorubicin treatment. (C) Inhibition of over-expressed HA-tagged CBP and flag-tagged p53 interaction in 293T cells by ischemin in a concentration-dependent manner under doxorubicin-induced DNA damaging condition. An arrow indicates the expressed Flag-tagged p53 in the HEK 293T cells.

FIG. 3. TUNEL assay showing doxorubicin induced p53 apoptosis in rat primary cardiomyocytes as visualized by the presences of nicks (green) in DNA. The latter is identified by terminal deoxynucleotidyl transferase that addes dUTPs to 3′-OH end of DNA and labeled with FITC for visualization.

FIG. 4. Ischemin functions a cellular protective agent against myocardial ischemic stress. (A) TUNEL assay showing ischemin inhibition of doxorubicin-induced apoptosis in rat neonatal cardiomyocytes. (B) Evaluation of ischemin effects in U20S cells and cardiomyocytes. The immunoblots show down-regulation of doxorubicin-induced activated p53 in both cell types in the presence of ischemin, while levels of H2XS139p remained the same. (C) Inhibition of doxorubicin-induced caspase 3/7 activation in cardiomyocytes by ischemin.

FIG. 5. BRD inhibitors down regulate TNFa-induced NF-kB activation. A. NF-kB activation by TNFa (10 ng/mL). HEK 293 cells (105/well) in a 24-well plate were stabilized with NF-kB response element (NF-kB_RE) was treated with TNF. Twenty-four hours after the treatment, the cells were harvested and lysed, and luciferase activity was determined. B. Dose-dependent inhibition of NF-kB activation by MS0129433 and MS0129436 (compounds of formula (1) and (2)).

FIG. 6 illustrates the inhibition of melanoma cell proliferation by MS0129436 (CM436).

FIG. 7 illustrates the inhibition of melanoma cell proliferation by CM225 and CM279 as compared to MS0129436 (CM436).

### DETAILED DESCRIPTION

For the terms “for example” and “such as,” and grammatical equivalences thereof, the phrase “and without limitation” is understood to follow unless explicitly stated otherwise. As used herein, the term “about” is meant to account for variations due to experimental error. All measurements reported herein are understood to be modified by the term “about”, whether or not the term is explicitly used, unless explicitly stated otherwise. As used herein, the singular forms “a,” “an,” and “the” include plural referents unless the context clearly dictates otherwise.

A “patient,” as used herein, includes both humans and other animals, particularly mammals. Thus the methods are applicable to both human therapy and veterinary applications. In some embodiments, the patient is a mammal, for example, a primate. In some embodiments, the patient is a human.

The terms “treating” and “treatment” mean causing a therapeutically beneficial effect, such as ameliorating existing symptoms, preventing additional symptoms, ameliorating or preventing the underlying metabolic causes of symptoms, postponing or preventing the further development of a disorder and/or reducing the severity of symptoms that will or are expected to develop.

A “therapeutically effective” amount of the compounds described herein is typically one which is sufficient to achieve the desired effect and may vary according to the nature and severity of the disease condition, and the potency of the compound. It will be appreciated that different concentrations may be employed for prophylaxis than for treatment of an active disease.

The term “contacting” means bringing at least two moieties together, whether in an in vitro system or an in vivo system.

The term “bioisostere” means a substituent that is believed to impart similar biological properties to a compound as an identified substituent. Accordingly, a hydroxy bioisostere, as used herein, refers to a substituent that is believed to impart similar biological properties as a hydroxyl moiety to the compounds described herein in conjunction with the phenyl ring on which it resides.

In general, reference to a certain element such as hydrogen or H is meant to include all isotopes of that element. For example if a R group is defined to represent hydrogen or H, it also includes deuterium and tritium.

The term “alkyl” includes straight-chain alkyl groups (e.g., methyl, ethyl, propyl, butyl, pentyl, hexyl, heptyl, octyl, nonyl, decyl, etc.) and branched-chain alkyl groups (isopropyl, tert-butyl, isobutyl, etc.), cycloalkyl (alicyclic) groups (cyclopropyl, cyclopentyl, cyclohexyl, cycloheptyl, cyclooctyl), alkyl substituted cycloalkyl groups, and cycloalkyl substituted alkyl groups. In certain embodiments, a straight chain or branched chain alkyl has 10 or fewer carbon atoms in its backbone (e.g., C₁₋₁₀ for straight chain, C₃₋₁₀ for branched chain). The term C₁₋₁₀ includes alkyl groups containing 1 to 10 carbon atoms.

The term “cycloalkyl” includes a cyclic aliphatic group which may be saturated or unsaturated. For example, cycloalkyl groups include cyclopropyl, cyclopentyl, cyclohexyl, cycloheptyl, and cyclooctyl. In some embodiments, cycloalkyls have from 3-8 carbon atoms in their ring structure, for example, they can have 3, 4, 5 or 6 carbons in the ring structure.

In general, the term “aryl” includes groups, including 5- and 6-membered single-ring aromatic groups, such as benzene and phenyl. Furthermore, the term “aryl” includes multicyclic aryl groups, e.g., tricyclic, bicyclic, such as naphthalene and anthracene.

The term “heteroaryl” includes groups, including 5- and 6-membered single-ring aromatic groups, that have from one to four heteroatoms, for example, pyrrole, furan, thiophene, thiazole, isothiaozole, imidazole, triazole, tetrazole, pyrazole, oxazole, isooxazole, pyridine, pyrazine, pyridazine, and pyrimidine, and the like. Furthermore, the term “heteroaryl” includes multicyclic heteroaryl groups, e.g., tricyclic, bicyclic, such as benzoxazole, benzodioxazole, benzothiazole, benzoimidazole, benzothiophene, methylenedioxyphenyl, quinoline, isoquinoline, napthyridine, indole, benzofuran, purine, benzofuran, quinazoline, deazapurine, indazole, or indolizine.

The term “heterocycloalkyl” includes groups, including but not limited to, 3- to 10-membered single or multiple rings having one to five heteroatoms, for example, piperazine, pyrrolidine, piperidine, or homopiperazine.

The term “substituted” means that an atom or group of atoms formally replaces hydrogen as a “substituent” attached to another group. For aryl and heteroaryl groups, the term “substituted”, unless otherwise indicated, refers to any level of substitution, namely mono, di, tri, tetra, or penta substitution, where such substitution is permitted. The substituents are independently selected, and substitution may be at any chemically accessible position. In some cases two sites of substitution may come together to form a 3-10 membered cycloalkyl or heterocycloalkyl ring.

As used herein, “administration” refers to delivery of a compound or composition as described herein by any external route, including, without limitation, IV, intramuscular, SC, intranasal, inhalation, transdermal, oral, buccal, rectal, sublingual, and parenteral administration.

Compounds described herein, including pharmaceutically acceptable salts thereof, can be prepared using known organic synthesis techniques and can be synthesized according to any of numerous possible synthetic routes.

The reactions for preparing the compounds described herein can be carried out in suitable solvents which can be readily selected by one of skill in the art of organic synthesis. Suitable solvents can be substantially non-reactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of compounds can involve the protection and deprotection of various chemical groups. The need for protection and deprotection, and the selection of appropriate protecting groups, can be readily determined by one skilled in the art. The chemistry of protecting groups can be found, for example, in Protecting Group Chemistry, 1^{st} Ed., Oxford University Press, 2000; and March's Advanced Organic chemistry: Reactions, Mechanisms, and Structure, 5^{th} Ed., Wiley-Interscience Publication, 2001 (each of which is incorporated herein by reference in their entirety).

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatographic methods such as high performance liquid chromatography (HPLC), liquid chromatography-mass spectroscopy (LCMS) or thin layer chromatography (TLC). Compounds can be purified by those skilled in the art by a variety of methods, including high performance liquid chromatography (HPLC) (“Preparative LC-MS Purification: Improved Compound Specific Method Optimization” K. F. Blom, et al., J. Combi. Chem. 6(6) (2004), which is incorporated herein by reference in its entirety) and normal phase silica chromatography.

### Compounds of Formula (1):

Provided herein are compounds of formula (1):

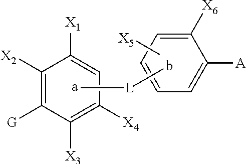

or a pharmaceutically acceptable salt form thereof, wherein: A is selected from the group consisting of:

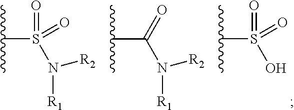

L is a linking group selected from:

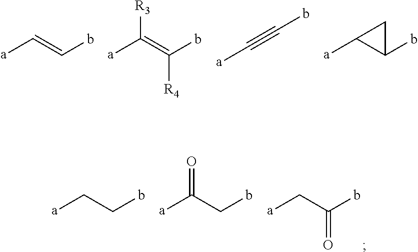

G is a heteroatom-containing group capable of accepting a hydrogen bond or donating a hydrogen bond, or G is fused to X₂ or X₃ to form a heterocyclic ring system capable of accepting or donating a hydrogen bond;

X₁ and X₄ are independently selected from the group consisting of: H, C₁₋₁₀ alkyl, Cl₁₋₁₀ perfluoroalkyl, halogen, nitrile, hydroxy, C₁₋₁₀ alkoxy, C₁₋₁₀ perfluoroalkoxy, C₁₋₁₀ thioalkyl, C₁₋₁₀ perfluoroalkyl, amine, alkylamino, C₁₋₁₀ acylamino, aryl, heteroaryl, carboxamido, carboxyl, and carboalkoxy;

X₂ and X₃ are independently selected trom the group consisting of: H, C₁₋₁₀ alkyl, C₁₋₁₀ perfluoroalkyl, halogen, nitrile, hydroxy, C₁₋₁₀ alkoxy, C₁₋₁₀ perfluoroalkoxy, C₁₋₁₀ thioalkyl, C₁₋₁₀ perfluoroalkyl, amine, alkylamino, C₁₋₁₀ acylamino, aryl, heteroaryl, carboxamide, and C₂₋₁₀ acyl;

optionally, X₁ and X₂ may come together to form a cycloalkyl, heterocycloalkyl, aromatic or heteroaromatic ring system;

X₅ and X₆ are independently selected from the group consisting of: H, C₁₋₁₀ alkyl, C₁₋₁₀ alkoxy, C₁₋₁₀ perfluoroalkyl, halogen, and nitrile;

R₁ is selected from the group consisting of: substituted or unsubstituted aryl, substituted or unsubstituted heteroaryl, and substituted or unsubstituted C₁₋₁₀ alkyl;

R₂ is selected from the group consisting of: H and C₁₋₁₀ alkyl;

optionally, R₁ and R₂ may come together to form a substituted or unsubstituted heterocycloalkyl ring system; and

R₃ and R₄ are independently selected from the group consisting of: H and C₁₋₁₀ alkyl.

In some embodiments, A is:

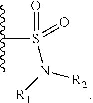

In some embodiments, L is selected from the group consisting of:

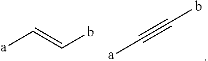

G can be any suitable heteroatom-containing group capable of accepting a hydrogen bond or donating a hydrogen bond. For example, G can be selected from OH, CH₂OH, NH₂, SH, C(O)H, CO₂H, OC(O)HCN, NHC(O)H, NH(SO₂)H, NHC(O)NH₂, NHCN, CH(CN)₂, F, Cl, OSO₃H, ONO₂H, and NO₂. In some embodiments, G is OH or an OH bioisostere (e.g., CH₂OH, NH₂, SH, NHC(O)H, NH(SO₂)H, NHC(O)NH₂, NHCN, and CH(CN)₂). In some embodiments, G is fused to X₂ or X₃ to form a heterocyclic ring system capable of accepting or donating a hydrogen bond. For example, a heterocyclic ring system can be selected from: azetidinyl, pyrrolyl, imidazolyl, pyrazolyl, pyridinyl, pyrazinyl, pyrimidinyl, pyridazinyl, indolizinyl, isoindolyl, indolyl, dihydroindolyl, indazolyl, furanyl, purinyl, quinolizinyl, isoquinolinyl, quinolinyl, phthalazinyl, naphthylpyridinyl, quinoxalinyl, quinazolinyl, cinnolinyl, pteridinyl, carbazolyl, carbolinyl, phenanthridinyl, acridinyl, phenanthrolinyl, isothiazolyl, phenazinyl, isoxazolyl, phenoxazinyl, phenothiazinyl, imidazolidinyl, imidazolinyl, imidazolyl, piperidinyl, piperazinyl, indolinyl, phthalimidyl, 1,2,3,4-tetrahydroisoquinolinyl, 4,5,6,7-tetrahydrobenzo[b]thiophenyl, thiazolyl, thiazolidinyl, thiophenyl, benzo[b]thiophenyl, morpholino, thiomorpholino, piperidinyl, pyrrolidinyl, and tetrahydrofuranyl.

For example, a compound of formula (1) can be a compound of formula (1A):

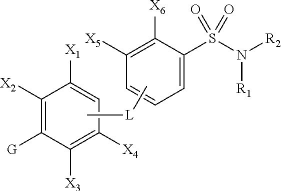

or a pharmaceutically acceptable salt form thereof, wherein: L is selected from the group consisting of:

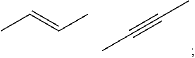

G is selected from OH, CH₂OH, NH₂, SH, C(O)H, CO₂H, OC(O)HCN, NHC(O)H, NH(SO₂)H, NHC(O)NH₂, NHCN, CH(CN)₂, F, Cl, OSO₃H, ONO₂H, and NO₂, or G is fused to X₂ to form a heterocyclic ring system capable of accepting or donating a hydrogen bond;
X₁ is a protected or unprotected amine;
X₂ and X₃ are independently selected from the group consisting of: H, C₁₋₁₀ alkyl, halogen;

### X₄, X₅, and X₆ are H;

R₁ is selected the group consisting of: substituted C₁₋₁₀ alkyl, aryl, and heteroaryl;

### R₂ is H.

In some embodiments, G is OH or an OH bioisostere as described above. For example, G can be OH.

Non-limiting examples of a compound of formula (1) include:

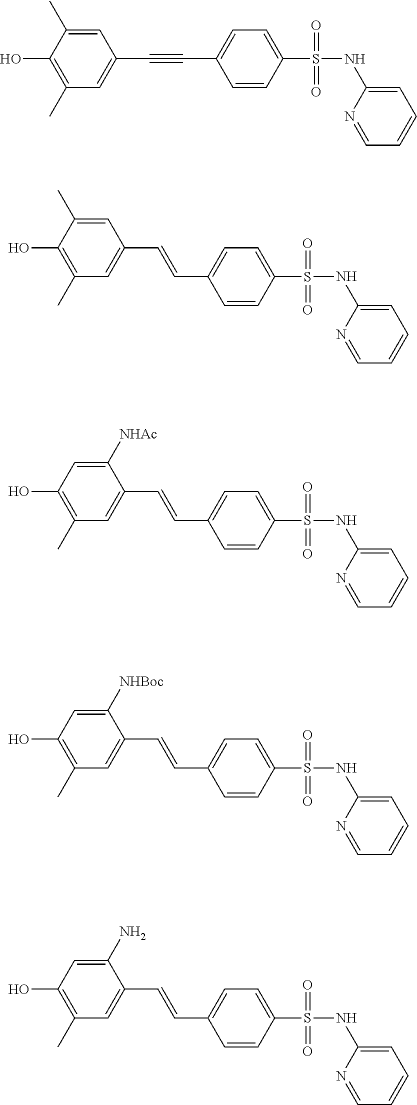 [SMILES: Cc1cc(C#Cc2ccc(S(=O)(=O)Nc3ccccn3)cc2)cc(C)c1O ; Cc1cc(/C=C/c2ccc(S(=O)(=O)Nc3ccccn3)cc2)cc(C)c1O ; CC(=O)Nc1cc(O)c(C)cc1/C=C/c1ccc(S(=O)(=O)Nc2ccccn2)cc1 ; Cc1cc(/C=C/c2ccc(S(=O)(=O)Nc3ccccn3)cc2)c(NC(=O)OC(C)(C)C)cc1O ; Cc1cc(/C=C/c2ccc(S(=O)(=O)Nc3ccccn3)cc2)c(N)cc1O]

or a pharmaceutically acceptable salt form thereof.

A compound of formula (1) can be prepared, for example, as shown in Scheme 1 and described in Example 1.

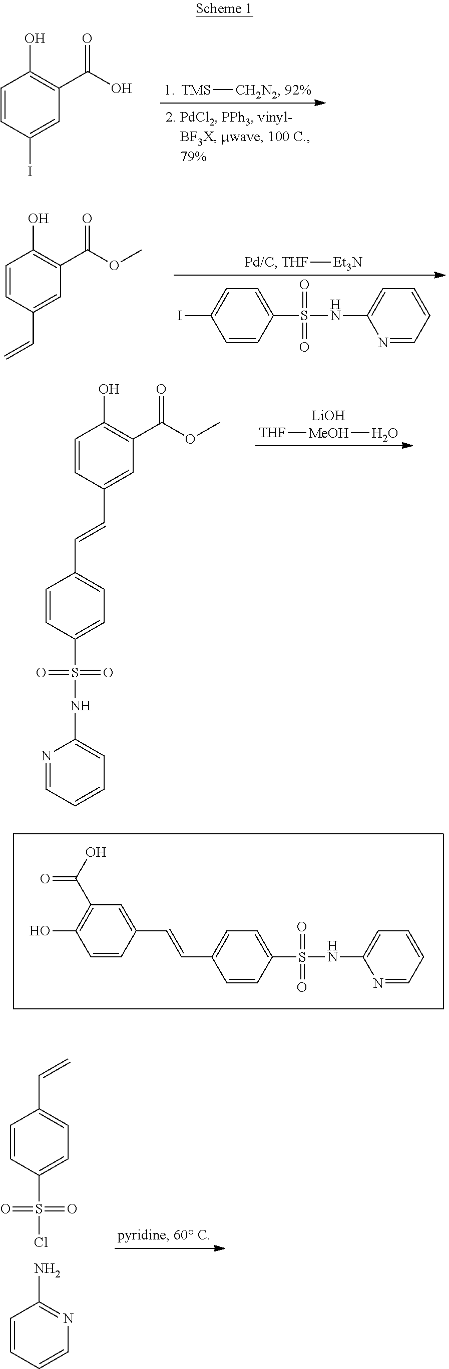

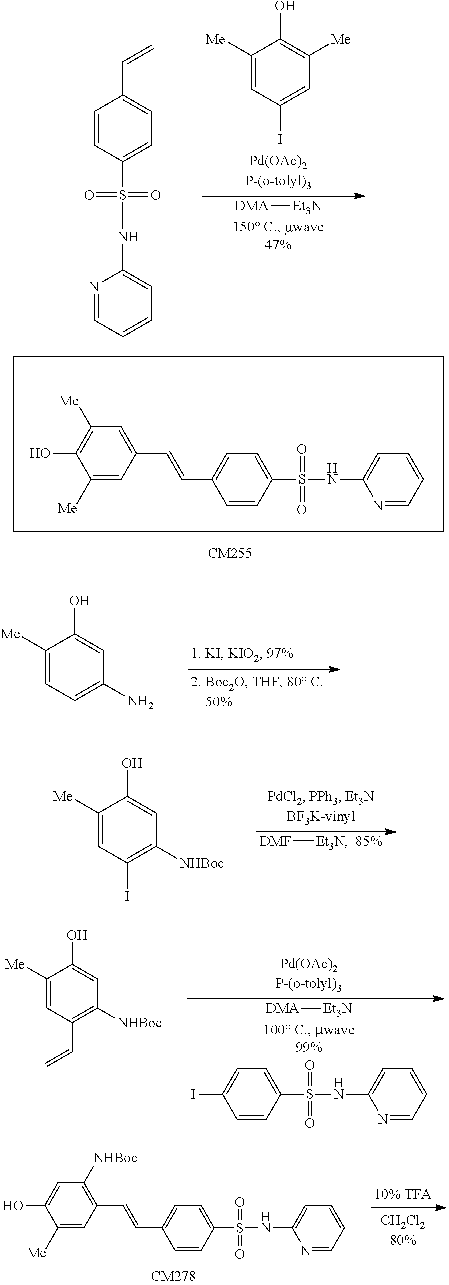

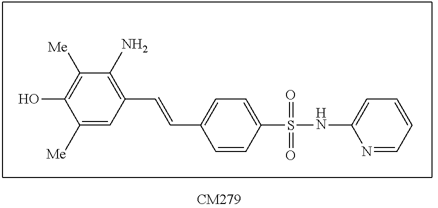 [SMILES: Cc1cc(/C=C/c2ccc(S(=O)(=O)Nc3ccccn3)cc2)c(N)c(C)c1O]

### Compounds of Formula (2):

Also provided herein are compounds of formula (2):

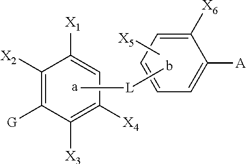

or a pharmaceutically acceptable salt form thereof, wherein:

A is selected from the group consisting of:

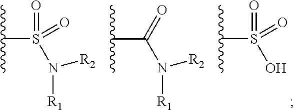

L is:

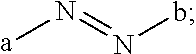

G is a heteroatom containing group capable of accepting a hydrogen bond or donating a hydrogen bond, or G is fused to X₂ or X₃ to form a heterocyclic ring system capable of accepting or donating a hydrogen bond;

X₁ and X₄ are independently selected from the group consisting of: H, C₁₋₁₀ alkyl, C₁₋₁₀ perfluoroalkyl, halogen, nitrile, hydroxy, C₁₋₁₀ alkoxy, C₁₋₁₀ perfluoroalkoxy, C₁₋₁₀ thioalkyl, C₁₋₁₀ perfluoroalkyl, amine, alkylamino, C₁₋₁₀ acylamino, aryl, heteroaryl, carboxamido, carboxyl, and carboalkoxy;

X₂ and X₃ are independently selected from the group consisting of: H, C₁₋₁₀ alkyl, C₁₋₁₀ perfluoroalkyl, halogen, nitrile, hydroxy, C₁₋₁₀ alkoxy, C₁₋₁₀ perfluoroalkoxy, C₁₋₁₀ thioalkyl, C₁₋₁₀ perfluoroalkyl, amine, alkylamino, C₁₋₁₀ acylamino, aryl, heteroaryl, carboxamide, and C₂₋₁₀ acyl;

optionally, X₁ and X₂ may come together to form a cycloalkyl, heterocycloalkyl, aromatic or heteroaromatic ring system;

X₅ and X₆ are independently selected from the group consisting of: H, C₁₋₁₀ alkyl, C₁₋₁₀ alkoxy, C₁₋₁₀ perfluoroalkyl, halogen, and nitrile;

R₁ is selected from the group consisting of: substituted or unsubstituted aryl, substituted or unsubstituted heteroaryl, and substituted or unsubstituted C₁₋₁₀ alkyl;

R₂ is selected from the group consisting of: H and C₁₋₁₀ alkyl;

optionally, R₁ and R₂ may come together to form a substituted or unsubstituted heterocycloalkyl ring system; and

R₃ and R₄ are independently selected from the group consisting of: H and C₁₋₁₀ alkyl.

In some embodiments, A is:

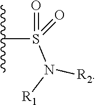

G can be any suitable heteroatom-containing group capable of accepting a hydrogen bond or donating a hydrogen bond. For example, G can be selected from OH, CH₂OH, NH₂, SH, C(O)H, CO₂H, OC(O)HCN, NHC(O)H, NH(SO₂)H, NHC(O)NH₂, NHCN, CH(CN)₂, F, Cl, OSO₃H, ONO₂H, and NO₂. In some embodiments, G is OH or an OH bioisostere (e.g., CH₂OH, NH₂, SH, NHC(O)H, NH(SO₂)H, NHC(O)NH₂, NHCN, and CH(CN)₂). In some embodiments, G is fused to X₂ or X₃ to form a heterocyclic ring system capable of accepting or donating a hydrogen bond. For example, a heterocyclic ring system can be selected from: azetidinyl, pyrrolyl, imidazolyl, pyrazolyl, pyridinyl, pyrazinyl, pyrimidinyl, pyridazinyl, indolizinyl, isoindolyl, indolyl, dihydroindolyl, indazolyl, furanyl, purinyl, quinolizinyl, isoquinolinyl, quinolinyl, phthalazinyl, naphthylpyridinyl, quinoxalinyl, quinazolinyl, cinnolinyl, pteridinyl, carbazolyl, carbolinyl, phenanthridinyl, acridinyl, phenanthrolinyl, isothiazolyl, phenazinyl, isoxazolyl, phenoxazinyl, phenothiazinyl, imidazolidinyl, imidazolinyl, imidazolyl, piperidinyl, piperazinyl, indolinyl, phthalimidyl, 1,2,3,4-tetrahydroisoquinolinyl, 4,5,6,7-tetrahydrobenzo[b]thiophenyl, thiazolyl, thiazolidinyl, thiophenyl, benzo[b]thiophenyl, morpholino, thiomorpholino, piperidinyl, pyrrolidinyl, and tetrahydrofuranyl.

For example, a compound of formula (2) can be a compound of formula (2A):

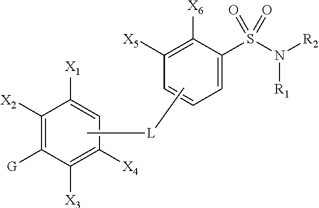

or a pharmaceutically acceptable salt form thereof, wherein:

L is:

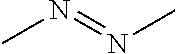

G is selected from OH, CH₂OH, NH₂, SH, C(O)H, CO₂H, OC(O)HCN, NHC(O)H, NH(SO₂)H, NHC(O)NH₂, NHCN, CH(CN)₂, F, Cl, OSO₃H, ONO₂H, and NO₂;

X₁ is H or a protected or unprotected amine;

X₂ and X₃ are independently selected from the group consisting of: H, halogen, hydroxyl, C₁₋₁₀ alkyl, C₁₋₁₀ perfluoroalkyl, and C₁₋₁₀ alkoxy;

X₄ is H;

X₅ and X₆ are independently selected from the group consisting of: H, halogen, hydroxyl, C₁₋₁₀ alkyl, and C₁₋₁₀ alkoxy;

R₁ is selected the group consisting of: substituted C₁₋₁₀ alkyl, aryl, and heteroaryl; and

R₂ is H.

In some embodiments, A is:

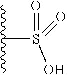 [SMILES: *S(=O)(=O)O]

In some embodiments, G is OH or an OH bioisostere, as described above. For example, G can be OH.

For example, a compound of formula (2) can be a compound of formula (2B):

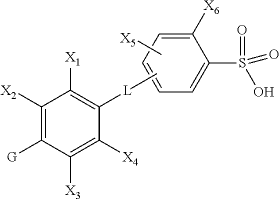

or a pharmaceutically acceptable salt form thereof, wherein:

L is:

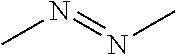

G is OH;

X₁ and X₄ are H;

X₂ and X₃ are independently selected from the group consisting of: H, halogen, hydroxyl, C₁₋₁₀ alkyl, C₁₋₁₀ perfluoroalkyl, and C₁₋₁₀ alkoxy; and

X₅ and X₆ are independently selected from the group consisting of: H, halogen, hydroxyl, C₁₋₁₀ alkyl, and C₁₋₁₀ alkoxy.

Non-limiting examples of a compound of formula (2) include:

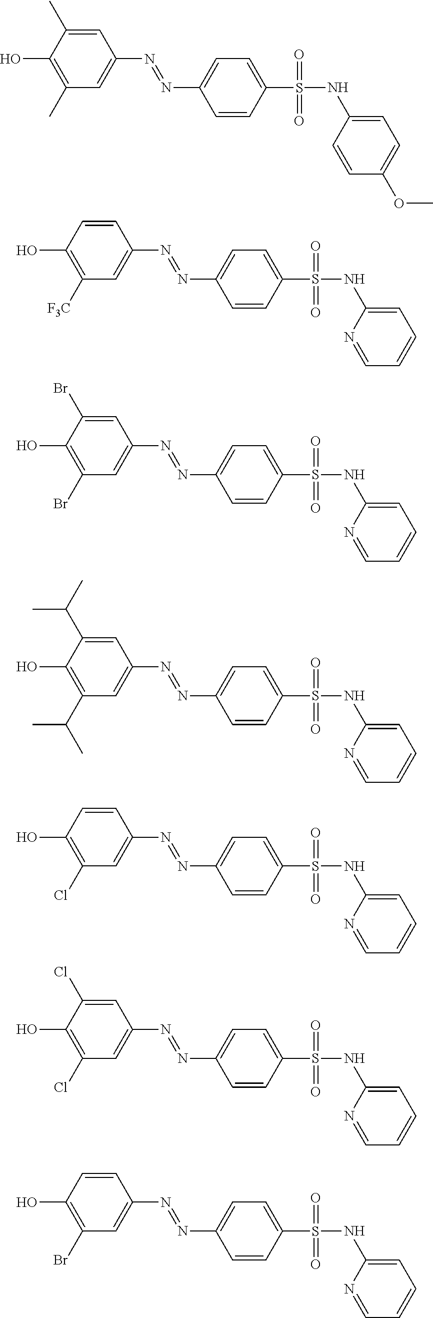

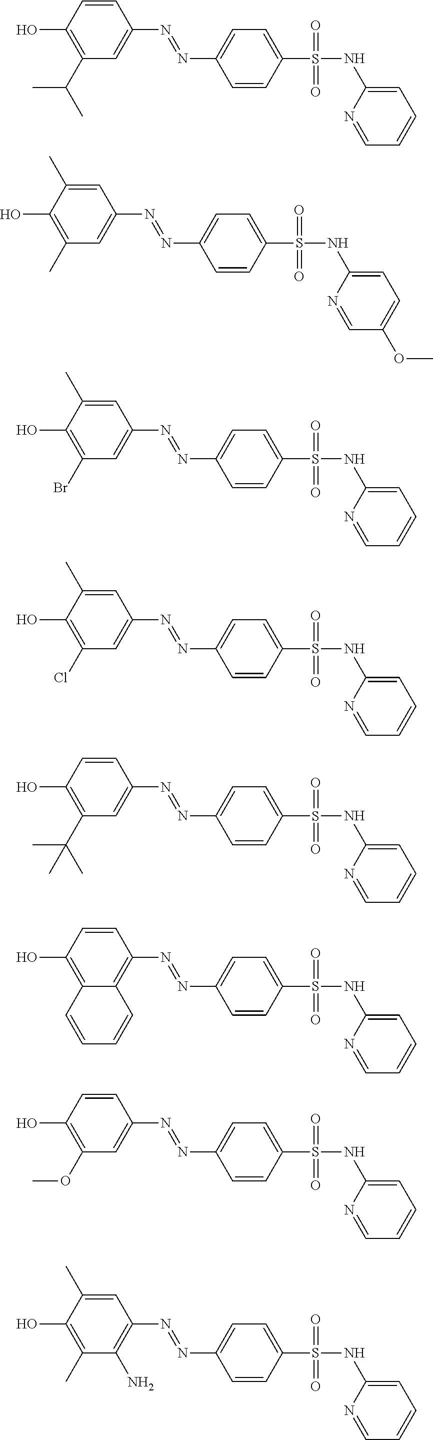

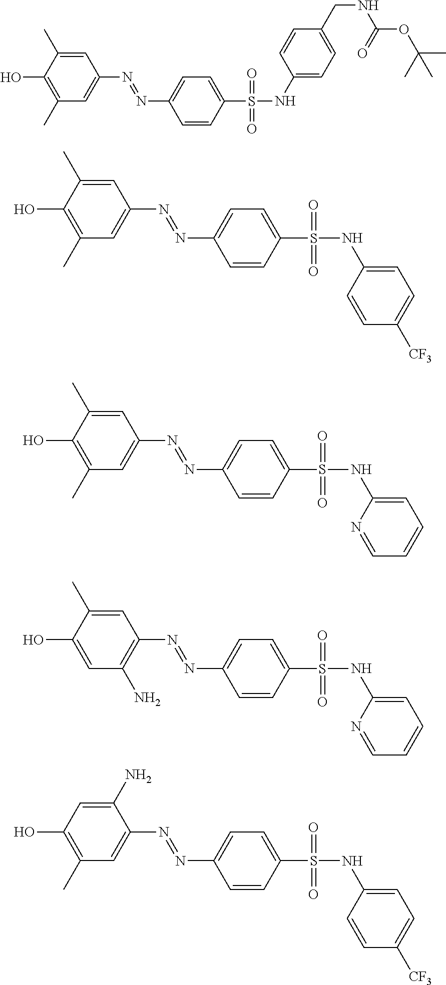

A compound of formula (2) can be prepared, tor example, as described in Examples 2-4.

### Pharmaceutically Acceptable Salts and Compositions

Pharmaceutically acceptable salts of the compounds described herein include the acid addition and base salts thereof.

Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, hydrogen phosphate, isethionate, D- and L-lactate, malate, maleate, malonate, mesylate, methylsulphate, 2-napsylate, nicotinate, nitrate, orotate, oxalate, palmitate, pamoate, phosphate/hydrogen, phosphate/phosphate dihydrogen, pyroglutamate, saccharate, stearate, succinate, tannate, D- and L-tartrate, 1-hydroxy-2-naphthoate tosylate and xinafoate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulphate and hemicalcium salts.

Compounds described herein intended for pharmaceutical use may be administered as crystalline or amorphous products. They may be obtained, for example, as solid plugs, powders, or films by methods such as precipitation, crystallization, freeze drying, spray drying, or evaporative drying. Microwave or radio frequency drying may be used for this purpose.

The compounds may be administered alone or in combination with one or more other compounds described herein or in combination with one or more other drugs (or as any combination thereof). Generally, they will be administered as a formulation in association with one or more pharmaceutically acceptable excipients. The term “excipient” is used herein to describe any ingredient other than the compound(s) of the invention. The choice of excipient will to a large extent depend on factors such as the particular mode of administration, the effect of the excipient on solubility and stability, and the nature of the dosage form.

Non-limiting examples of pharmaceutical excipients suitable for administration of the compounds provided herein include any such carriers known to those skilled in the art to be suitable for the particular mode of administration. Pharmaceutically acceptable excipients include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β, and γ-cyclodextrin, or chemically modified derivatives such as hydroxyalkylcyclodextrins, including 2- and 3-hydroxypropyl-b-cyclodextrins, or other solubilized derivatives can also be advantageously used to enhance delivery of compounds of the formulae described herein. In some embodiments, the excipient is a physiologically acceptable saline solution.

The compositions can be, in one embodiment, formulated into suitable pharmaceutical preparations such as solutions, suspensions, tablets, dispersible tablets, pills, capsules, powders, sustained release formulations or elixirs, for oral administration or in sterile solutions or suspensions for parenteral administration, as well as transdermal patch preparation and dry powder inhalers (see, e.g., Ansel Introduction to Pharmaceutical Dosage Forms, Fourth Edition 1985, 126).

The concentration of a compound in a pharmaceutical composition will depend on absorption, inactivation and excretion rates of the compound, the physicochemical characteristics of the compound, the dosage schedule, and amount administered as well as other factors known to those of skill in the art.

The pharmaceutical composition may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular patient, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

The pharmaceutical compositions are provided for administration to humans and animals in unit dosage forms, such as tablets, capsules, pills, powders, granules, sterile parenteral solutions or suspensions, and oral solutions or suspensions, and oil-water emulsions containing suitable quantities of the compounds or pharmaceutically acceptable derivatives thereof. The pharmaceutically therapeutically active compounds and derivatives thereof are, in one embodiment, formulated and administered in unit-dosage forms or multiple-dosage forms. Unit-dose forms as used herein refer to physically discrete units suitable for human and animal patients and packaged individually as is known in the art. Each unit-dose contains a predetermined quantity of the therapeutically active compound sufficient to produce the desired therapeutic effect, in association with the required pharmaceutical carrier, vehicle or diluent. Examples of unit-dose forms include ampoules and syringes and individually packaged tablets or capsules. Unit-dose forms may be administered in fractions or multiples thereof A multiple-dose form is a plurality of identical unit-dosage forms packaged in a single container to be administered in segregated unit-dose form. Examples of multiple-dose forms include vials, bottles of tablets or capsules or bottles of pints or gallons. Hence, multiple dose form is a multiple of unit-doses which are not segregated in packaging.

Liquid pharmaceutically administrable compositions can, for example, be prepared by dissolving, dispersing, or otherwise mixing an active compound as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, solubilizing agents, pH buffering agents and the like, for example, acetate, sodium citrate, cyclodextrine derivatives, sorbitan monolaurate, triethanolamine sodium acetate, triethanolamine oleate, and other such agents.

Dosage forms or compositions containing a compound as described herein in the range of 0.005% to 100% with the balance made up from non-toxic carrier may be prepared. Methods for preparation of these compositions are known to those skilled in the art. The contemplated compositions may contain 0.001%400% active ingredient, in one embodiment 0.1-95%, in another embodiment 75-85%.

Pharmaceutical compositions suitable for the delivery of compounds described herein and methods for their preparation will be readily apparent to those skilled in the art. Such compositions and methods for their preparation may be found, for example, in Remington's Pharmaceutical Sciences, 19th Edition (Mack Publishing Company, 1995).

### Methods of Use

The compounds and compositions provided herein can be used as to block the acetyl-lysine binding activity of a bromodomain containing transcriptional co-activator, transcription regulator protein, or chromatin remodeling regulator protein. See, for example, Examples 5-8. Such inhibition can lead to attenuated gene transcriptional activity that induces or contributes to the disease or disorder. In some embodiments, a compound as described herein makes hydrogen bond contacts with an acetyl-lysine binding asparagine residue of a bromodomain containing transcriptional co-activator, transcription regulator protein, or chromatin remodeling regulator protein. This bonding can lead to attenuated transcriptional activity that induces or contributes to the disease or disorder being treated.

The transcriptional co-activator, transcription regulator protein, or chromatin remodeling regulator protein can include one or more of PCAF, GCN5L2, p300/CBP, TAF1, TAF1L, Ash1L, MLL, SMARCA2, SMARCA4, BRPF1, ATAD2, BRD7, BRD2, BRD3, BRD4, BRDT, BAZ1B (WSTF), BAZ2B, BPTF, SP140L, TRIM24, and TRIM33.

In some embodiments, the transcriptional activity of NF-kB and its target genes are modulated. The compounds and compositions described herein can be useful in the treatment of diseases where NF-kB is over activated. In some embodiments, the transcriptional activity of human p53 and activation of its target genes are modulated by the compounds and compositions provided herein. Accordingly, the compounds and compositions can be useful in the treatment of disease or condition wherein p53 activity is hyper-activated under a stress-induced event such as trauma, hyperthermia, hypoxia, ischemia, stroke, a burn, a seizure, a tissue or organ prior to transplantation, or a chemo- or radiation therapy treatment. In some embodiments, the transcriptional activity of transcription co-activators CBP/p300 by binding to the bromodomain is modulated by the compounds and compositions provided herein. For example, the compounds and compositions can be useful in the treatment of disease or condition wherein CBP/p300 activity is inducing or promoting the disease or condition including cancer, acute myeloid leukemia (AML), chronic myeloid leukemia, circadian rhythm disorders, or drug addiction. In some embodiments, the transcriptional activity of Williams-Beuren syndrome transcription factor (WSTF) by binding to the bromodomain is modulated by the compounds and compositions provided herein. In some cases, the compounds and compositions are useful in the treatment of disease or condition wherein WSTF hyper-activity in over-expressed vitamin A receptor complexes is implicated, for example, in cancer of the breast, head and neck, and lungs, as well as leukemia and skin cancers.

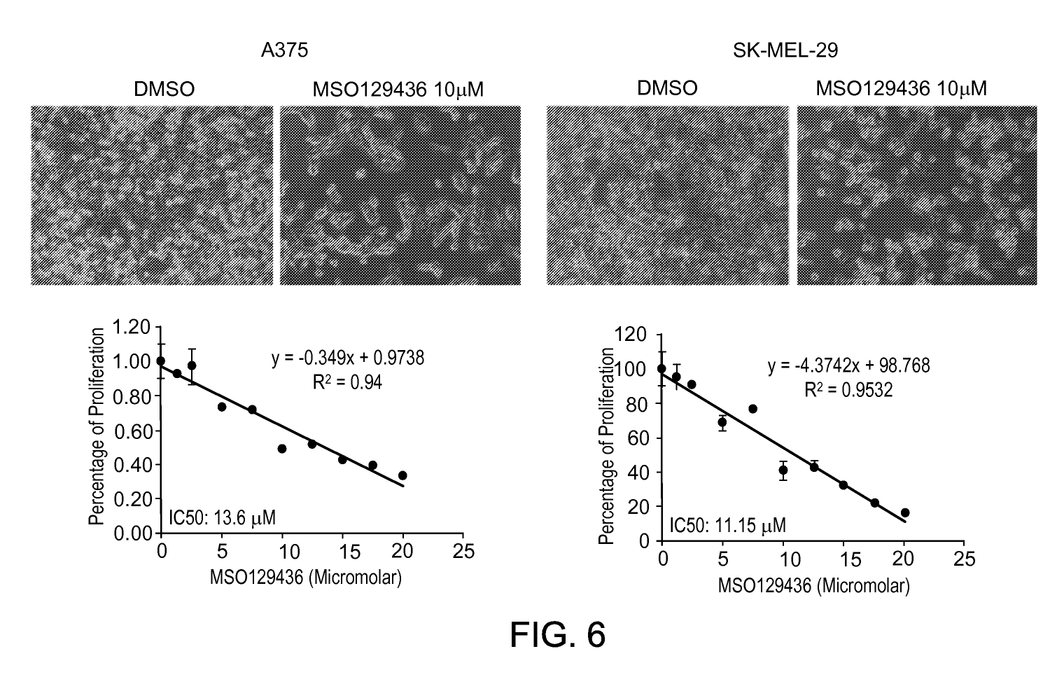

For example, in melanoma, metastatic potential and aggressiveness correlates with NF-kB over expression (see, e.g., J. Yang, Richmond Cancer Research 61:4901-4909 (2001); and Ryu, B. et al., PLoS ONE 7:e595 (July 2007). As is shown in FIG. 6, MS0129436 inhibits proliferation of melanoma cells in vitro but has no effect on mormal melanocytes. MS0129436 has the structure:

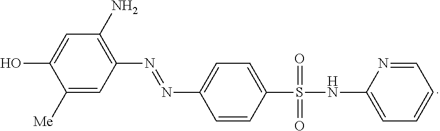 [SMILES: Cc1cc(/N=N/c2ccc(S(=O)(=O)Nc3ccccn3)cc2)c(N)cc1O]

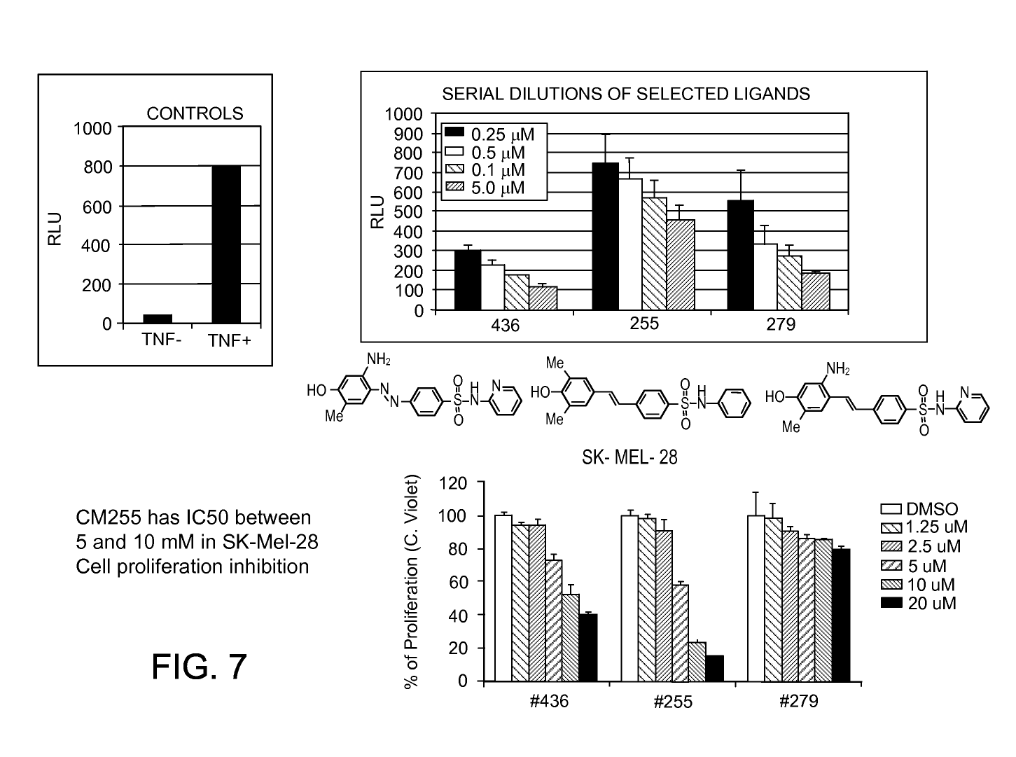

As shown in FIG. 7, compounds of formula (1), e.g., CM255 and CM279, are further capable of inhibiting melanoma cell proliferation.

Non-limiting examples of diseases which can be treated with the compounds and compositions provided herein include a variety of cancers, inflammatory diseases, neurological disorders, and viral infections (e.g., HIV/AIDS).

The biological activity of the compounds described herein can be tested using any suitable assay known to those of skill in the art. For example, the activity of a compound may be tested using one or more of the methods described in Example 5-8.

Non-limiting examples of such data are shown in the following tables.

TABLE 1
Structure-Activity Relationship Data of Bromodomain Inhibitors

| | Binding Affinity, Kd (μM) | | | |
|---|---|---|---|---|
| Compounds | PCAF | CBP | BRD4-1 | BRD4-2 |
| 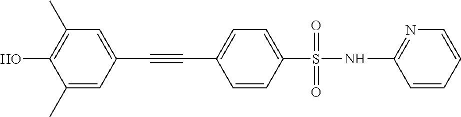 [SMILES: Cc1cc(C#Cc2ccc(S(=O)(=O)Nc3ccccn3)cc2)cc(C)c1O] CM0000254 | N/A | N/A | <1 uM | 1.6 |
| 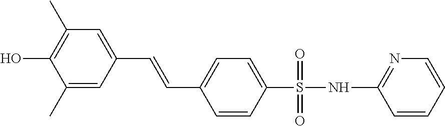 [SMILES: Cc1cc(/C=C/c2ccc(S(=O)(=O)Nc3ccccn3)cc2)cc(C)c1O] CM0000255 | <1 uM | 32.8 | <1 uM | <1 uM |
| 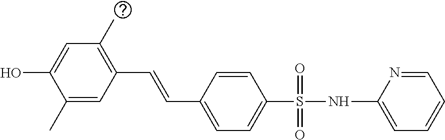 [SMILES: Cc1cc(/C=C/c2ccc(S(=O)(=O)Nc3ccccn3)cc2)c(C)cc1O] CM0000277 | N/A | N/A | <1 uM | N/A |
| 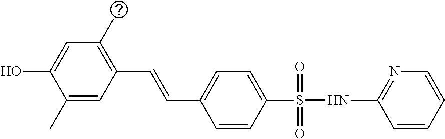 [SMILES: Cc1cc(/C=C/c2ccc(S(=O)(=O)Nc3ccccn3)cc2)c(C)cc1O] CM0000278 | N/A | N/A | 1.5 | <1 uM |
| 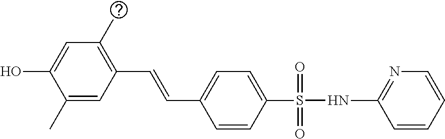 [SMILES: Cc1cc(/C=C/c2ccc(S(=O)(=O)Nc3ccccn3)cc2)c(C)cc1O] CM0000279 | N/A | N/A | 2.1 | <1 uM |

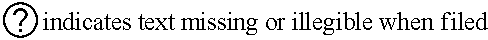

TABLE 2
Structure-Activity Relationship Data of Bromodomain Inhibitors

| | Binding Affinity, Kd (μM) | | | |
|---|---|---|---|---|
| Compounds | PCAF | CBP | BRD4-1 | BRD4-2 |
| 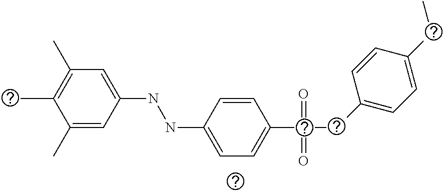 | 60.3 | N/A | 32.6 | 17 8 |
| 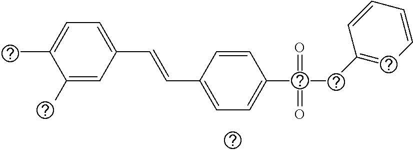 | 260.2 | 276.6 | 69.5 | 102. [symbol] |
| 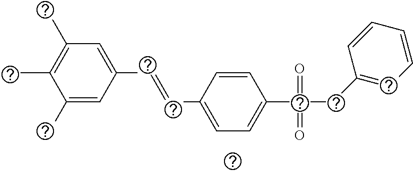 | 31.0 | 120. [symbol] | 111 0 | 19.3 |
| 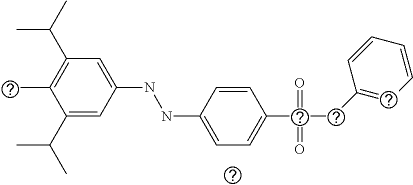 | 38. [symbol] | N/A | 505.2 | 44 9 |
| 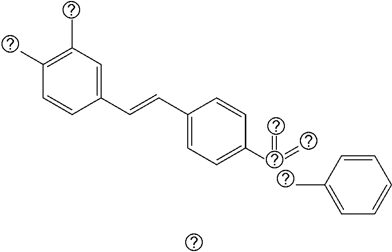 | N/A | 588.0 | 15 7 | 120.3 |
| 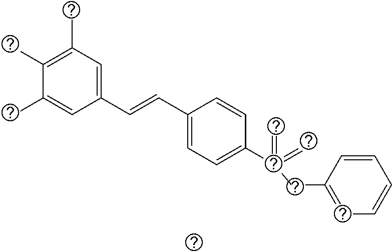 | 11.1 | 1,025.0 | 10.0 | 27.0 |
| 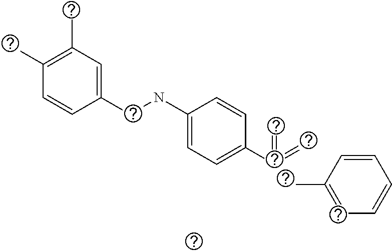 | 193.3 | N/A | 107 5 | 286.7 |
| 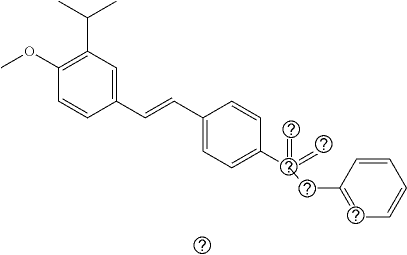 | N/A | N/A | N/A | 26.4 |
| 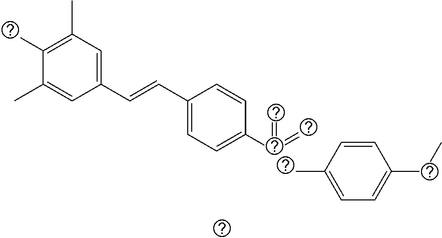 | 5 8 | N/A | 13 6 | 14.4 |
| 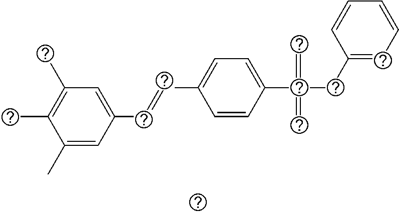 | 5.6 | N/A | 6.7 | 4.5 |
| 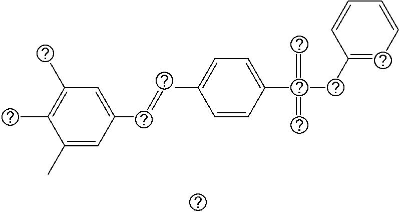 | 25 2 | N/A | 9.1 | 20.5 |
| 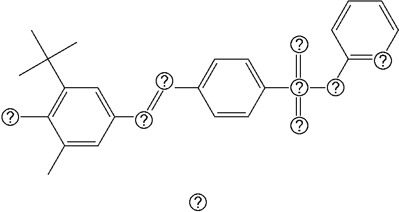 | N/A | N/A | [symbol] .0 | 29 4 |
| 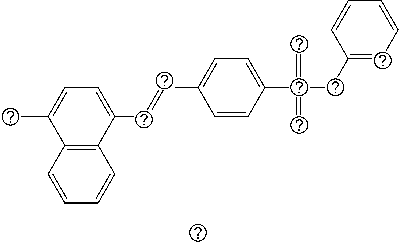 | 6 5 | N/A | 5 6 | 6.1 |
| 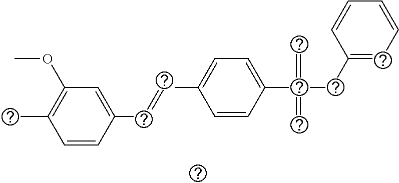 | N/A | N/A | 4.8 | 10.2 |
| 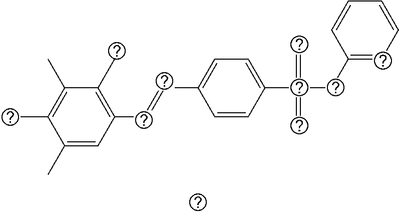 | <1 uM | 1.3 | <1 uM | <1 uM |
| 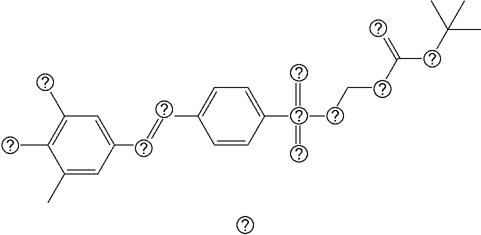 | N/A | N/A | <1 uM | N/A |
| 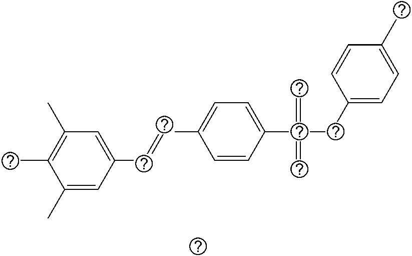 | N/A | N/A | 3.5 | N/A |
| 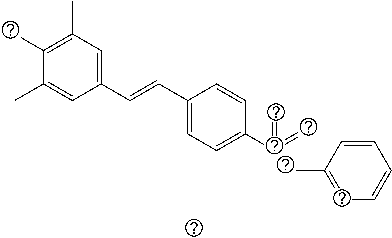 | 10 1 | N/A | 5.4 | [symbol] .2 |
| 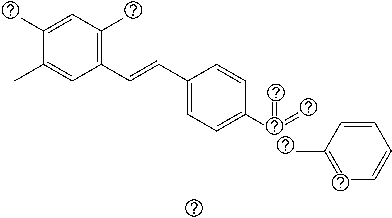 | 5.8 | 2.6 | 5.8 | 4.0 |
| 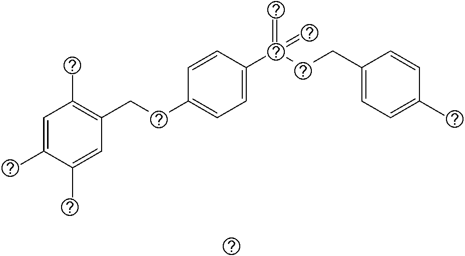 | N/A | N/A | 4.5 | 21.0 |

[symbol]

indicates data missing or illegible when filed

[symbol]

TABLE 3
Structure-Activity Relationships of Azobenzene Compounds in p53 Inhibition

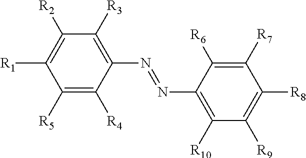

| | | | | | | | | | | | % |
|---|---|---|---|---|---|---|---|---|---|---|---|
| Compound | R1 | R2 | R3 | R4 | R5 | R6 | R7 | R8 | R9 | R10 | Inhibition |
| MS456 | OH | H | H | H | H | H | H | SO₃H | H | H | 4.6 |
| MS450 | OH | CH₃ | H | H | H | H | H | SO₃H | H | H | 85.6 |
| MS113 | OH | CH₃CH₂ | H | H | H | H | H | SO₃H | H | H | 25.7 |
| MS451 | OH | CH₃ | H | H | CH₃ | H | H | SO₃H | H | H | 87.4 |
| MS110 | OH | CH₂CHCH₂ | H | H | CH₃ | H | H | SO₃H | H | H | 86.2 |
| MS111 | OH | (CH₃)₃C | H | H | CH₃ | H | H | SO₃H | H | H | 22.8 |
| MS105 | OH | (CH₃)₂CH | H | H | (CH₃)₂CH | H | H | SO₃H | H | H | 26.4 |
| MS101 | OH | CH₃ | H | CH₃ | H | H | H | SO₃H | H | H | 82.9 |
| MS103 | OH | CH₃ | H | CH₃ | CH₃ | H | H | SO₃H | H | H | 38.9 |
| MS100 | OH | H | CH₃ | CH₃ | H | H | H | SO₃H | H | H | 36.4 |
| Ischemin/MS120 | OH | H | NH₂ | H | CH₃ | H | SO₃H | CH₃ | H | CH₃ | 104.5 |
| MS119 | OH | H | CH₃ | CH₃ | H | H | SO₃H | CH₃ | H | CH₃ | 54.0 |
| MS153 | OH | H | CH₃ | CH₃ | H | H | SO₃H | OH | Cl | H | 39.0 |
| MS131 | OH | Cl | H | H | H | H | SO₃H | CH₃ | H | CH₃ | 49.7 |
| MS124 | OH | CH₃CH₂ | H | H | H | H | SO₃H | CH₃ | H | CH₃ | 25.0 |
| MS126 | OH | CH₃CH₂CH₂ | H | H | H | H | SO₃H | CH₃ | H | CH₃ | 93.5 |
| MS127 | OH | CH₃CH₂CO | H | H | H | H | SO₃H | CH₃ | H | CH₃ | 86.8 |
| MS109 | OH | CH₃ | H | H | CH₃ | H | SO₃H | CH₃ | H | CH₃ | 60.1 |
| MS130 | OH | CH₂CHCH₂ | H | H | CH₃ | H | SO₃H | CH₃ | H | CH₃ | 40.4 |
| MS129 | OH | (CH₃)₂CH | H | H | CH₃ | H | SO₃H | CH₃ | H | CH₃ | 44.6 |
| MS128 | OH | (CH₃)₂CH | H | H | (CH₃)₂CH | H | SO₃H | CH₃ | H | CH₃ | 47.2 |
| MS135 | OH | NH₂ | H | CH₃ | H | H | SO₃H | CH₃ | H | CH₃ | 54.8 |
| MS118 | OH | CH₃ | H | CH₃ | H | H | SO₃H | CH₃ | H | CH₃ | 49.7 |
| MS146 | OH | CH₃ | H | CH₃ | CH₃ | H | SO₃H | CH₃ | H | CH₃ | 30.8 |

Notes:
1. All compounds were used at 50 μM concentrat:on.
2. Percent Inhibition was calculated by [1 − (A/B)]\hat{ }100. where A is the difference of luciferase activity measured between cells treated with a compound and doxorubicin and the negative control. and B is the difference of luciferase activity between cells treated with and without doxorubicin.
3. Compounds shown 80%+ inhibition of p53 activity are highlighted in blue.

TABLE 4
Bromodomain Binding is Retained in C═C Bridged Systems

| | | PCAF | CBP | BrD4-1 | BrD4-2 |
|---|---|---|---|---|---|
| Structure | Compound | (μM) | (μM) | (μM) | (μM) |
| 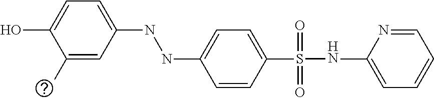 [SMILES: Cc1cc(NNc2ccc(S(=O)(=O)Nc3ccccn3)cc2)ccc1O] | Sulfasalazine (MS0123028) | 88 | 82 | 54 | 54 |
| 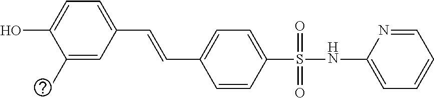 [SMILES: Cc1cc(/C=C/c2ccc(S(=O)(=O)Nc3ccccn3)cc2)ccc1O] | CM0000252 | 6 | 76 | 12 | 16 |
| 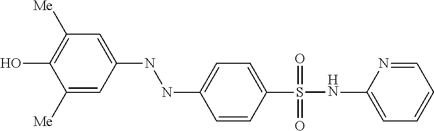 [SMILES: Cc1cc(NNc2ccc(S(=O)(=O)Nc3ccccn3)cc2)cc(C)c1O] | MS0129435 | 4.5 | >100 | 1.3 | 2.4 |
| 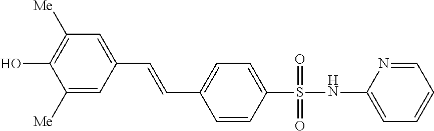 [SMILES: Cc1cc(/C=C/c2ccc(S(=O)(=O)Nc3ccccn3)cc2)cc(C)c1O] | CM0000255 | <1 | 33 | <1 | <1 |
| 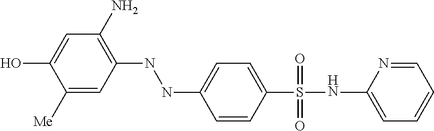 [SMILES: Cc1cc(NNc2ccc(S(=O)(=O)Nc3ccccn3)cc2)c(N)cc1O] | MS0129436 | 6.8 | 14.0 | 6.0 | 5.7 |
| 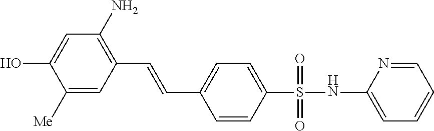 [SMILES: Cc1cc(/C=C/c2ccc(S(=O)(=O)Nc3ccccn3)cc2)c(N)cc1O] | CM0000279 | NA | NA | 2 | <1 |

Bromodomain binding is retained in C═C bridged systems-loss of PCAF and CBP affinity in CM0000279 vs MS0129436 is suprising

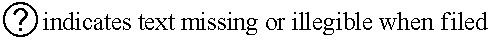

### EXAMPLES

### Example 1

### Preparation of a Compound of Formula (1)

### A. Procedures for Building Blocks and Intermediates for Compounds of Formula (1)

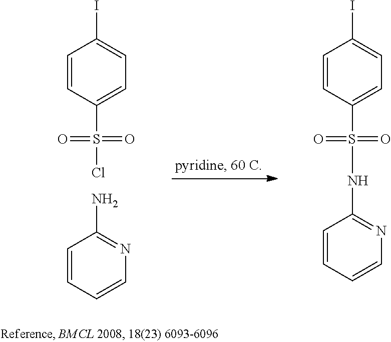

A solution of 2-aminopyridine (1.0 g, 10.6 mmol) in pyridine (5 mL) was cooled to 0° C. and treated with pipsyl chloride (3.37 g, 11.2 mmol) in several portions. The solution was heated to 60° C. for 1 h, then cooled to 25° C. The majority of solvent was removed in vacuo, and the residue was suspended in a minimal amount of MeOH (20 mL), and H₂O (100 mL). The white solid that had formed was collected by suction filtration. This solid was dissolved in a minimal amount of CH₂Cl₂ and precipitated by the addition of hexanes to afford the final compound as a white solid (3.34 g, 87%) that was used without further purification. ¹H NMR (600 MHz, DMSO-d₆) δ 7.97 (1H, d, J=4.8 Hz), 7.91 (2H, d, J=8.4 Hz), 7.75 (1H, t, J=7.2 Hz), 7.61 (2H, d, J=8.4 Hz), 7.16 (1H, d, J=8.4 Hz), 6.85 (1H, t, J=6.0 Hz). LCMS m/z 360.9686 ([M+H⁺], C₁₁H₉IN₂O₂S requires 360.9502). For reference the material runs to an approximate Rf of 0.5 in 1:1 EtOAc-hexanes).

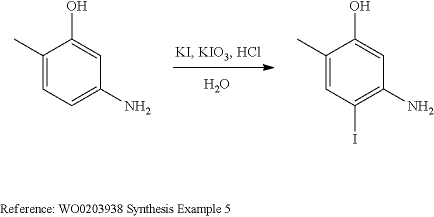

A solution of 5-amino cresol (3.08 grams, 25.0 mmol, 1 eq), was dissolved in H₂O (50 mL) and treated with concentrated HCl (2.06 mL, 37% solution, 25.0 mmol, 1 eq). This solution was cooled to 0° C. and treated dropwise with a combined solution of KI (2.77 g, 16.7 mmol, 0.66 eq) and KIO₃ (1.78 g, 8.33 mmol, 0.33 eq) dissolved in H₂O (25 mL). The solution was stirred for 1 h at 25° C. and then the brown solid that had formed was collected by suction filtration to afford 5-amino-4-iodo-2-methylphenol (6.04 g, 97%). The solid was dried on high vacuum overnight and used without further purification. (For reference the material runs to an approximate Rf of 0.6 in 10% EtOAc-hexanes). ¹H NMR (600 MHz, CDCl₃) δ 7.34 (1H, s), 6.26 (1H, s), 4.87 (2H, br s), 2.10 (3H, s). LCMS m/z 250.0634 ([M+H⁺], C₇H₈INO requires 249.9723)

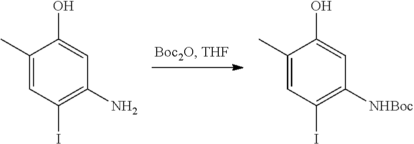

A solution of 5-amino-4-iodo-2-methylphenol (2.0 g, 8.03 mmol) in THF (10 mL) was treated with Boc₂O (2.63 g, 12.03 mmol, 1.5 eq) and heated to 80° C. for 14 h. The solution was cooled to 25° C., concentrated in vacuo and then purified by flash chromatography (0-15% EtOAc-hexanes). The purified fractions were combined, concentrated, and the residue was taken up in a minimal amount of Et₂O and treated with hexanes to afford the protected 5-amino-4-iodo-2-methylphenol as a white solid (1.39 g, 50%). ¹H NMR (600 MHz, CDCl₃) δ 7.51 (1H, s), 7.32 (1H, s), 6.62 (1H, br s), 2.03 (3H, s), 1.55 (9H, s). LCMS m/z 372.0167 ([M+Na⁺], C₁₂H₁₆INO₃ requires 372.0067).

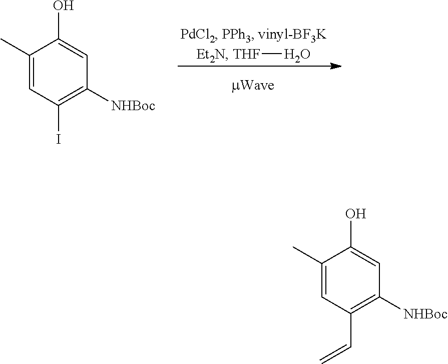

A solution of the starting material (1.0 g, 2.85 mmol) in 9:1 THF:H₂O (8.0 mL) was treated with PdCl₂ (0.010 g, 0.057 mmol, 0.02 eq), PPh₃ (0.045 g, 0.171 mmol, 0.06 eq), vinyl-BF₃K (0.381 g, 2.85 mmol, 1 eq) and Et₃N (1.18 mL, 8.55 mmol, 3 eq). The solution was heated to 120° C. in a microwave vial for 2 h. The solution was then filtered, concentrated, and purified by flash chromatography (0-10% EtOAc-hexanes) to afford the final product (0.604 g, 85%) as clear oil. ¹H NMR (600 MHz, CDCl₃) δ 7.13 (1H, s), 6.69 (1H, dd, J=6.2, 10.9 Hz), 6.42 (1H, br s), 5.53 (1H, d, J=17.4 Hz), 5.27 (1H, d, J=10.8 Hz), 2.18 (3H, s), 1.52 (9H, s). LCMS m/z 272.1821 ([M+Na⁺, C₁₂H₁₆INO₃ requires 272.1257).

### B. Example CM278

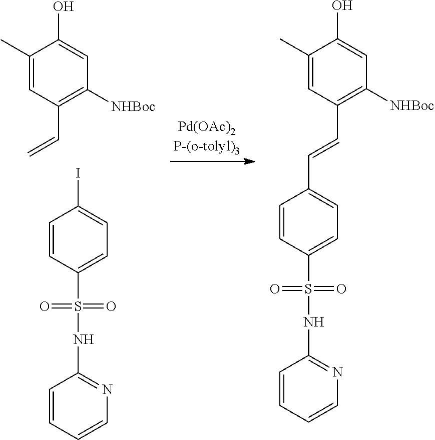

A solution of the starting material (0.807, 2.24 mmol, 1.1 eq, iodide) in 1:1 DMF:Et₃N (6.0 mL) was treated with Pd(OAc)₂ (0.091 g, 0.406 mmol, 0.02 eq), P(o-tolyl)₃ (0.371 g, 1.22 mmol, 0.06 eq), and alkene product (0.508 g, 2.03 mmol, 1 eq). The solution was heated to 100° C. in a microwave vial for 2 h. The solution was then filtered, concentrated in vacuo and purified by flash chromatography (0-3% MeOH—CH₂Cl₂) to afford CM278 (1.11 g, 99%) as clear oil. ¹H NMR (600 MHz, CDCl₃) δ 8.36 (1H, d, J=5.4 Hz), 7.89 (2H, d, J=8.4 Hz), 7.71 (1H, t, J=7.8 Hz), 7.53 (2H, d, J=8.4 Hz), 7.45 (1H, d, J=9.0 Hz), 7.30 (1H, s), 7.16 (1H, d, J=16.2 Hz), 6.86 (1H, d, J=16.2 Hz), 6.83 (1H, t, J=6.0 Hz), 6.52 (1H, s), 2.18 (3H, s), 1.51 (9H, s). LCMS m/z 482.1496 ([M+H⁺¹], C₂₅H₂₇N₃O₅S requires 482.1744).

### C. Example CM279

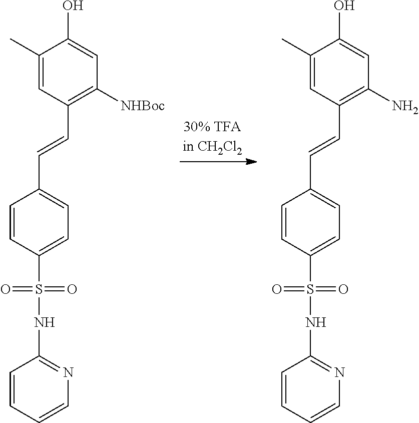 [SMILES: Cc1cc(/C=C/c2ccc(S(=O)(=O)Nc3ccccn3)cc2)c(NC(=O)OC(C)(C)C)cc1O ; Cc1cc(/C=C/c2ccc(S(=O)(=O)Nc3ccccn3)cc2)c(N)cc1O]

A solution of the starting material (0.613 g, 1.28 mmol) in CH₂Cl₂ (10.0 mL) was cooled to 0° C. and treated slowly and dropwise with trifluoroacetic acid (3.0 mL). The solution was warmed to 25° C., stirred for 1 h, and then concentrated under a stream of N₂. The crude material was dissolved in a minimal amount of CH₂Cl₂, and purified by flash chromatography (50% EtOAc-hexanes (to remove residual starting material and Iodide from the previous step), followed by 17:2:1 EtOAc-IPA-H₂O to elute the product. The fractions containing product were concentrated, taken up in a minimal amount of EtOAc-CH₂Cl₂ and precipitated by the dropwise addition of hexanes to afford xx as a gold solid (0.181 g, 37%) and a brown oil (0.208 g, 43%). ¹H NMR (600 MHz, CD₃OD) δ 7.98 (1H, d, J=4.8 Hz), 7.84 (2H, d, J=7.8 Hz), 7.61 (2H, d, J=8.4 Hz), 7.41 (1H, d, J=15.6 Hz), 7.22 (1H, d, J=8.4 Hz), 7.21 (1H, s), 6.88 (1H, m), 6.85 (1H, d, J=16.2 Hz), 2.04 (3H, s). LCMS m/z 382.1228 ([M+H⁺], C₂₀H₁₉N₃O₃S requires 382.1220.

### D. Example CM255

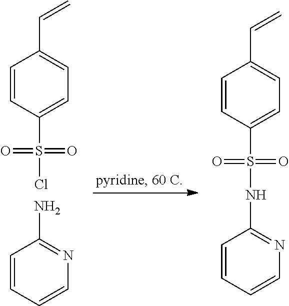

A solution of 2-aminopyridine (5.0 g, 53.1 mmol) in pyridine (20.0 mL) was cooled to 0° C. and treated dropwise with p-styrene sulfonyl chloride (8.6 mL, 55.8 mmol). The solution was heated to 60° C. for 1 h, then cooled to 25° C. and concentrated in vacuo. The residue was dissolved in EtOAc (500 mL) washed with 1 M aqueous HCl (2×200 mL), saturated aqueous NaCl (200 mL), dried (Na₂SO₄) and concentrated in vacuo. The crude residue was purified by flash chromatography (SiO₂, 0-3% MeOH—CH₂Cl₂). The pure fractions were combined, concentrated, taken up in minimal amount of EtOAc and precipitated by the addition of hexanes to afford the product as a white solid (10.4 g, 75%). ¹H NMR (600 MHz, CDCl₃) δ 8.33 (1H, d, J=4.8 Hz), 7.88 (2H, d, J=8.4 Hz), 7.69 (1H, td, J=7.2, 1.8 Hz), 7.48 (2H, d, J=8.4 Hz). 7.42 (1H, d, J=9.0 Hz), 6.82 (1H, t, J=6.6 Hz), 6.72 (1H, dd, J=6.6, 10.8 Hz), 5.84 (1H, d, J=18.0 Hz), 5.39 (1H, d, J=10.8 Hz). LCMS m/z 261.1192 ([M+H⁺], C₁₃H₁₂N₂O₂S requires 261.0692.)

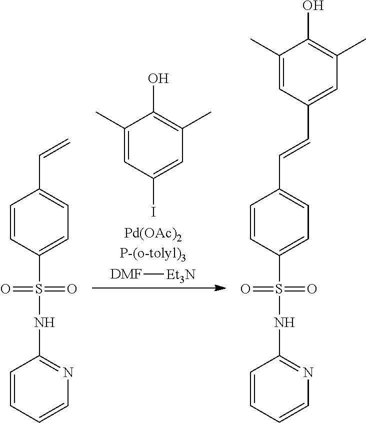

A solution of the starting material (1.00 g, 3.84 mmol) in 1:1 dimethyl acetamide:Et₃N (10 mL) was treated with Pd(OAc)₂ (0.172 g, 0.768 mmol), P(o-tolyl)₃ (0.701 g, 2.30 mmol), and 2,6-dimethyl-4-iodophenol (1.80 g, 7.68 mmol). The combined solution was degassed with a stream of Ar(g) for several minutes, the vial was then capped and heated to 150° C. (μwave) for 2 h. The vial was cooled to 25° C. and the solution was filtered through a pad of Celite. The organic solution was diluted into EtOAc (500 mL), washed with saturated aqueous NaCl (3×100 mL), dried (Na₂SO₄), and concentrated. The residue was then purified by flash chromatography (SiO₂, 30-60% hex-EtOAc, followed by 3% MeOH—CH₂Cl₂ to recover additional, albeit less pure, material which was later repurified by the same column conditions). The pure fractions from the EtOAc-hexanes eluent were concentrated, then taken up in a minimal amount of EtOAc and precipitated by the addition of hexanes to afford CM255 as a white solid (0.691 g, 47%). ¹H NMR (600 MHz, CD₃OD) δ 7.98 (1H, d, J=4.8 Hz), 7.85 (2H, d, J=8.4 Hz), 7.70 (1H, t, J=7.2 Hz), 7.59 (2H, d, J=8.4 Hz), 7.25 (1H, d, J=9.0 Hz), 6.96 (1H, d, J=16.2 Hz), 7.15 (2H, s), 7.14 (1H, d, J=16.2 Hz), 6.88 (1H, t, J=6.6 Hz), 2.18 (6H, s). LCMS m/z 382.1535 ([M+H⁺], C₂₁H₂₀N₂O₃S requires 381.1267).

### E. Example CM377

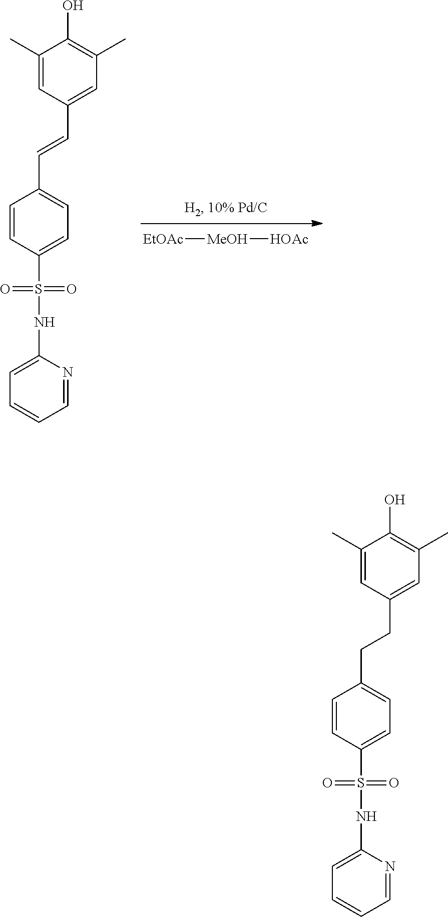

A solution of the starting material (0.100 g, 2.63 mmol) in 2:1:1 ethyl acetate:methanol:acetic acid (4.0 mL) was treated with 10% Pd/C (20 mg) and stirred vigorously under one atmosphere of H₂ (g) for 2 h. The mixture was filtered through Celite, and concentrated. The residue was dissolved in a minimal amount of ethyl acetate, and precipitated by slow addition of hexanes to afford CM377 as a white solid (0.716 g, 71%). ¹H NMR (600 MHz, CD₃OD) δ 7.97 (1H, d, J=4.8 Hz), 7.80 (2H, d, J=8.4 Hz), 7.69 (1H, td, J=8.4, 1.2 Hz), 7.26 (2H, d, J=8.4 Hz), 7.21 (1H, d, J=9.0 Hz), 6.88 (1H, t, J=6.0 Hz), 6.64 (2H, s), 2.88 (2H, t, J=7.2 Hz), 2.72 (2H, t, J=7.2 Hz), 2.11 (6H, s). LCMS m/z 383.1732 ([M+H⁺], C₂₁H₂₂N₂O₃S requires 383.1424).

### F. Example CM254

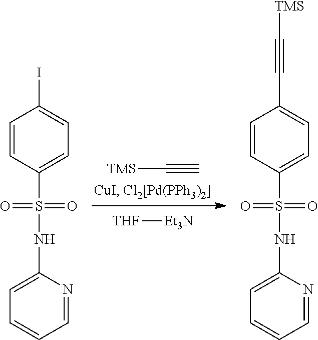

A solution of the starting material (2.0 g, 5.55 mmol) in 1:1 THF:Et₃N (27 mL) was treated with CuI (0.053 g, 0.278 mmol), Cl₂[Pd(PPh₃)₂] (0.195 g, 0.278 mmol), and TMS-alkyne (1.04 mL, 7.49 mmol). The combined solution was degassed with a stream of argon for several minutes, the vial capped, and heated to 70° C. for 14 h. The mixture was cooled to 25° C., filtered, concentrated and purified by flash chromatography (SiO₂, 33-50% EtOAc-hexanes) to afford the product as a white solid (1.57 g, 86%). ¹H NMR (600 MHz, CDCl₃) δ 8.34 (1H, d, J=6.0 Hz), 7.85 (2H, d, J=8.4 Hz), 7.69 (1H, td, J=7.2, 1.8 Hz), 7.53 (2H, d, J=8.4 Hz), 7.36 (1H, d, J=9.0 Hz), 6.81 (1H, t, J=6.6 Hz), 0.22 (9H, s). LCMS m/z 331.2019 ([M+H⁺], C₁₆H₁₈N₂O₂SSi requires 331.0931).

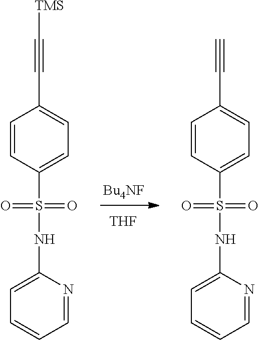 [SMILES: C[Si](C)(C)C#Cc1ccc(S(=O)(=O)Nc2ccccn2)cc1 ; C#Cc1ccc(S(=O)(=O)Nc2ccccn2)cc1 ; "THF"]

A solution of the starting material (1.57 g, 4.75 mmol) in THF (20 mL) was cooled to 0° C. and treated with a solution of Bu₄NF in THF (1.0 M, 5.0 mL). The combined solution was warmed to 25° C. and stirred for 1 h. The mixture was poured over saturated aqueous NaCl (100 mL) and extracted with EtOAc (3×200 mL). The combined organic layers were washed with saturated aqueous NaCl (2×100 mL), dried (Na₂SO₄), and concentrated in vacuo. The residue was taken up in a minimal amount of CH₂Cl₂ and purified by flash chromatography (SiO₂, 50% EtOAc-hexanes) to afford the product as a white solid (0.895 g, 73%). ¹H NMR (600 MHz, CDCl₃) δ 8.32 (1H, d, J=4.8 Hz), 7.89 (2H, d, J=8.4 Hz), 7.73 (1H, td, J=7.2, 1.8 Hz), 7.57 (2H, d, J=7.8 Hz), 7.42 (1H, d, J=9.0 Hz), 6.84 (1H, t, J=6.6 Hz), 3.22 (1H, s). LCMS m/z 259.0589 ([M+H⁺] C₁₃H₁₀N₂O₂S requires 259.0536).

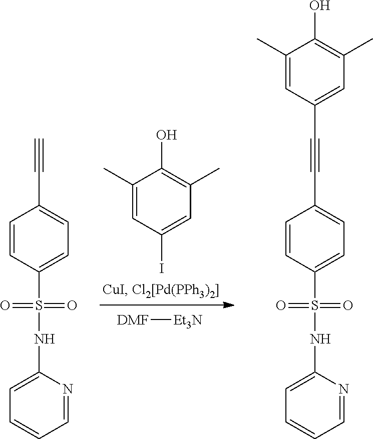

A solution of the starting material (0.050 g, 0.194 mmol) in 1:1 DMF:Et₃N (1.5 mL) was treated with CuI (0.0018 g, 0.0.0097 mmol), Cl₂[Pd(PPh₃)₂] (0.0.0068 g, 0.0097 mmol), and 2,6-dimethyl-4-iodophenol (0.053 g, 0.213 mmol). The combined solution was degassed with a stream of argon for several minutes, the vial was sealed and heated to 100° C. for 1 h in a microwave reactor. The mixture was cooled to 25° C. filtered, concentrated and purified by flash chromatography (SiO₂, 33-50% EtOAc-hexanes) to afford CM254 as a yellow solid (0.037 g, 50%). ¹H NMR (600 MHz, CDCl₃) δ 8.34 (1H, d, J=5.4 Hz), 7.88 (2H, d, J=8.4 Hz), 7.69 (1H, td, J=5.4, 1.8 Hz), 7.55 (2H, d, J=8.4 Hz), 7.40 (1H, d, J=9.0 Hz), 7.19 (2H, s), 6.81 (1H, t, J=6.6 Hz), 2.26 (6H, s). LCMS m/z 379.1233 ([M+H⁺], C₂₁H₁₈N₂O₃S requires 379.1111).

### Example 2

### Preparation of Compounds of Formula (2)

All reagents and solvents were obtained from commercial suppliers and used without further purification unless otherwise stated. Precoated silica gel plates (fluorescent indicator) were used for thin-layer analytical chromatography (Sigma-Aldrich) and compounds were visualized by LTV light or iodine. NMR spectra were recorded in deuterated solvents on a 600, 800 or 900 MHz Bruker NMR spectrometer and referenced internally to the residual solvent peak or TMS signals (δ_{H}=0.00 ppm, δ_{C}=0.00 ppm). Column chromatography was carried out employing Sigma-Aldrich silica gel (Kieselgel 60, 63-200 μm). MS (ESI) analysis was performed on LC-MS Aligent Technologies 1200 series.

### A. General Procedures for the Preparation of Azobenzene Compounds

Azobenzene compounds of formula (2) were synthesized using a two-step reaction procedure (Scheme 2). Specifically, the synthesis starts with treatment of a substituted sulfanilic acid (0.2 g, 1.154 mmol) with 5 ml of concentrated HCl and 1 g of crushed ice, and then cooled to 0° C. The resulting amine was diazotized by addition of 1 mL sodium nitrite to produce diazonium salt. After 2 hours diazonium salt was added drop-wise to a well-stirred, cold (0° C.) solution containing a substituted phenol (1.27 mmol) in 20 mL Aq. NaOH (10%). During the addition, the pH was kept above 8 by the periodic addition of cold (0° C.) 10% NaOH. After completion of the reaction pH of the solution was adjusted to 7 with 10% HCl, to give a yellow precipitate of a corresponding diazobenzene compound that was collected by filtration. The crude product was purified by column chromatography using DCM/MeOH (10%) as an eluent. For few compounds washing with proper solvent provided highly pure compounds (70-90% yield). For all compounds predominantly (E)-isomer were formed (>98% E)

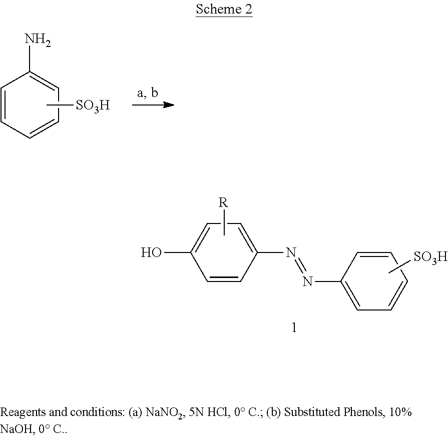

### B. Detailed Synthesis for the Individual Azobenzene Compounds

### 5-(2-amino-4-hydroxy-5-methylphenylazo)-2,4-dimethylbenzenesulfonic acid (Ischemin)

5-Amino-2,4-xylenesulfonic acid (0.23 g, 1.154 mmol) was mixed with 5 mL of concentrated HCl and 1 g of crushed ice, and then cooled to 0° C. The amine was diazotized by adding 1 mL of 1 N NaNO₂ with vigorous stirring. After 2 hours diazonium salt was added drop-wise to a well-stirred, cold (0° C.) solution containing 5-amino-2-methyl phenol (0.155 g, 1.27 mmol) in 20 mL Aq. NaOH (10%). During the addition, the pH was kept above 8 by the periodic addition of cold (0° C.) 10% NaOH. After completion of the reaction pH of the solution was adjusted to 7 with 10% HCl, to give a yellow precipitate that was collected by filtration. The crude product was purified by Column chromatography using DCM/MeOH (10%) as an eluent to afford the compound Ischemin (or MS 120) (0.327 g, 76.9% yield, 99% E-isomer). ¹H NMR (Methanol-d₄, 600 MHz) δ=8.11 (s, 1H), 7.55 (s, 1H), 7.16 (s, 1H), 6.80 (s, 1H), 2.60 (s, 3H), 2.59 (s, 3H), 2.55 (s, 3H). ¹³C NMR (800 MHz, MeOD) δ=155.5, 148.0, 144.2, 141.5, 139.4, 139.2, 138.6, 134.0, 119.3, 117.5, 116.8, 114.4, 19.6, 16.0, 15.9. MS (ESI) 336.11 (M⁺+1).

### 4-(4-hydroxy-2,6-dimethyl-phenylazo)-benzenesulfonic acid (MS100)

Compound (MS100) was obtained as a yellowish solid (70%). ¹HNMR (Methanol-d₄, 600 MHz) δ=8.10 (d, 2H), 7.98 (d, 2H), 6.71 (s, 2H), 2.64 (s, 6H); ¹³C NMR (900 MHz, MeOD) δ=164.4, 154.6, 144.8, 141.2, 136.7, 126.4, 121.0, 117.4, 19.9. MS (ESI) 307.08 (M⁺+1).

### 4-(4-hydroxy-2,5-dimethyl-phenylazo)-benzenesulfonic acid (MS101)

Compound (MS101) was obtained as a yellowish solid (70%). ¹HNMR (Methanol-d₄, 600 MHz) δ=7.82-7.83 (d, 2H, J=6 Hz), 7.70-7.72 (d, 2H, J=12 Hz), 7.48 (s, 1H), 6.51 (s, 1H), 2.49 (s, 3H), 2.07 (s, 3H); ¹³C NMR (800 MHz, MeOD) δ=154.5, 144.3, 141.6, 140.4, 136.6 126.4, 124.8, 121.2, 117.8, 117.2, 16.0, 15.3; MS (ESI) 307.08 (M⁺+1).

### 4-(4-hydroxy-2,3,5-trimethyl-phenylazo)-benzenesulfonic acid (MS103)

Compound (MS103) was obtained as a yellowish solid (78%). ¹HNMR (DMSO-d₆, 600 MHz) δ=7.80-7.81 (d, 2H, J=6 Hz), 7.77-7.79 (d, 2H, J=6 Hz), 6.7 (s, 1H), 2.66 (s, 3H), 2.22 (s, 3H), 2.13 (s, 3H); MS (ESI) 321.3 (M⁺+1).

### 4-(4-hydroxy-3,5-diisopropyl-phenylazo)-benzenesulfonic acid (MS105)

Compound (MS105) was obtained as a yellowish solid (76%). ¹HNMR (Methanol-d₄, 600 MHz) δ=8.10-8.12 (d, 2H, J=12 Hz), 8.01-8.02 (d, 2H, J=6 Hz), 7.4 (s, 2H) 3.52-3.57 (m, 2H), 1.43-1.44 (d, 12H); ¹³C NMR (900 MHz, MeOD) δ=168.3, 154.5, 143.5, 142.0, 137.7, 126.4, 120.6, 119.7, 26.1, 22.2. MS (ESI) 363.3 (M⁺+1).

### 5-(3,5-dimethyl-4-hydroxyphenylazo)-2,4-dimethylbenzenesulfonic acid (MS109)

Compound (MS109) was obtained as a yellowish solid (74%). ¹H NMR (Methanol-d₄, 600 MHz) δ=8.33 (s, 1H), 7.71 (s, 2H), 7.40 (s, 1H), 2.84 (s, 3H), 2.82 (s, 3H), 2.45 (s, 6H); ¹³C NMR (900 MHz, MeOD) δ=151.1, 143.4, 137.7, 135.1, 133.7, 132.6, 130.4, 129.5, 110.5, 108.5, 29.3, 22.6, 21.1. MS (ESI) 335.13 (M⁺+1).

### 4-(4-hydroxy-3-methyl-5-propene-phenylazo)-benzenesulfonic acid (MS110)

Compound (MS110) was obtained as a yellowish solid (76%). ¹HNMR (Methanol-d₄, 600 MHz)=8.09-8.10 (d, 2H, J=6 Hz), 7.97-7.99 (d, 2H, J=12 Hz), 7.6 (s, 2H) 6.19-6.26 (m, 1H), 5.21-5.27 (m, 2H), 3.60-3.61 (d, 2H, J=6 Hz), 2.45 (s, 3H). ¹³C NMR (800 MHz, MeOD) δ=150.9, 146.0, 145.1, 144.4, 135.6, 128.2, 126.6, 120.9, 119.8, 119.0, 96.2, 94.5, 43.8, 15.8. MS (ESI) 333.5 (M⁺+1).

### 4-(4-hydroxy-3-t-butyl-5-methyl-phenylazo)-benzenesulfonic acid (MS111)

Compound (MS111) was obtained as a yellowish solid (67%). ¹HNMR (Methanol-d₄, 600 MHz) δ=8.11-8.12 (d, 2H, J=6 Hz), 8.00-8.02 (d, 2H, J=12 Hz), 7.7 (s, 1H), 6.7 (s, 1H), 2.47 (s, 3H), 1.62 (s, 9H); ¹³C NMR (900 MHz, MeOD) δ=159.3, 153.0, 145.5, 145.0, 137.6, 126.5, 125.7, 123.2, 121.2, 34.4, 28.6, 15.6. MS (ESI) 349.5 (M⁺+1).

### 4-(4-hydroxy-3-ethyl-phenylazo)-benzenesulfonic acid (MS113)

Compound (MS113) was obtained as a yellowish solid (77%). ¹HNMR (Methanol-d₄, 600 MHz) δ=8.11-8.13 (d, 2H, J=12 Hz), 8.02-8.03 (d, 2H, J=6 Hz), 7.91 (s, 1H), 7.83-7.85 (d, 1H, J=12 Hz), 7.04-7.06 (d, 1H, J=12 Hz), 2.86 (q, 2H, J₁=24 Hz, J₂=6 Hz), 1.42 (t, 3H, J=6 Hz); ¹³C NMR (900 MHz, MeOD) δ=159.1, 153.6, 146.0, 145.8, 131.2, 126.5, 123.3, 123.0, 121.6, 114.5, 22.7, 12.9. MS (ESI) 307.5 (M⁺+1).

### 5-(2-amino-4-hydroxy-5-methoxy-phenylazo)-2,4-dimethylbenzenesulfonic acid (MS117)

Compound (MS117) was obtained as a yellowish solid (79%). ¹H NMR (Methanol-d₄, 600 MHz) δ=7.74 (s, 1H), 7.72 (s, 1H), 6.94 (s, 1H), 5.82 (s, 1H), 3.51 (s, 3H), 2.60 (s, 3H), 2.59 (s, 3H), MS (ESI) 352.44 (M⁺+1).

### 5-(3,6-dimethyl-4-hydroxyphenylazo-2,4-dimethylbenzenesulfonic acid (MS118)

Compound (MS118) was obtained as a yellowish solid (61%). ¹H NMR (Methanol-d₄, 600 MHz) δ=8.37 (s, 1H), 7.65 (s, 1H), 7.39 (s, 1H), 6.86 (s, 1H), 2.83 (s, 6H), 2.79 (s, 3H), 2.34 (s, 3H). ¹³C NMR (800 MHz, MeOD) δ=158.9, 148.6, 144.1, 141.4, 139.0, 138.4, 137.8, 133.8, 122.8, 117.8, 115.9, 114.5, 19.14, 16.0 (2C), 14.6. MS (ESI) 335.11 (M⁺+1).

### 5-(2,6-dimethyl-4-hydroxyphenylazo)-2,4-dimethylbenzenesulfonic acid (MS119)

Compound (MS119) was obtained as a yellowish solid (76.9%). ¹H NMR (Methanol-d₄, 600 MHz) δ=8.36 (s, 1H), 7.39 (s, 1H), 6.74 (s, 2H), 2.85 (s, 3H), 2.80 (s, 3H), 2.64 (s, 6H); ¹³C NMR (900 MHz, MeOD) δ=151.1, 143.4, 137.7, 135.1, 133.7, 132.6, 130.4, 129.5, 110.5, 108.5, 29.7, 29.1, 26.9. MS (ESI) 335.11 (M⁺+1).

### 4-(4-hydroxy-3-propyl-phenylazo)benzenesulfonic acid (MS123)

Compound (MS123) was obtained as a yellowish solid (74%). ¹H NMR (Methanol-d₄, 600 MHz) δ=7.87-7.89 (d, 2H, J=12 Hz), 7.77-7.79 (d, 2H, J=12 Hz), 7.63 (s, 1H), 7.58-7.59 (d, 1H, J=6 Hz), 6.80-6.81 (d, 1H, J=6 Hz), 2.56 (t, 2H, J=6 Hz), 1.59 (m, 2H), 1.15 (t, 3H, J=6 Hz). ¹³C NMR (800 MHz, MeOD) δ=159.4, 153.8, 146.1, 145.2, 129.6, 126.5, 124.6, 123.2, 121.9, 114.8, 31.9, 22.5, 13.1. MS (ESI) 349.7 (M⁺+1).

### 5-(3-ethyl-4-hydroxyphenylazo)-2,4-dimethylbenzenesulfonic acid (MS124)

Compound (MS124) was obtained as a yellowish solid (77%). ¹H NMR (Methanol-d₄, 600 MHz) δ=8.36 (s, 1H), 7.87 (s, 1H), 7.79-7.81 (d, 1H, J=12 Hz), 7.4 (s, 1H), 7.01-7.03 (d, 1H, J=12 Hz), 2.84 (s, 3H), 2.83 (s, 3H), 2.86-2.95 (m, 2H), 1.41 (t, 3H, J=6 Hz); ¹³C NMR (800 MHz, MeOD) δ=158.5, 148.2, 146.6, 141.4, 139.1, 138.1, 133.9, 131.1, 123.5, 122.2, 114.6, 114.0, 22.8, 19.1, 15.8, 13.0. MS (ESI) 335.13 (M⁺+1).

### 5-(4-hydroxy-3-propyl-phenylazo)-2,4-dimethylbenzenesulfonic acid (MS126)

Compound (MS126) was obtained as a yellowish solid (74%). ¹H NMR (Methanol-d₄, 600 MHz) δ=8.35 (s, 1H), 7.84 (s, 1H), 7.79-7.80 (d, 1H, J=6 Hz), 7.39 (s, 1H), 7.02-7.03 (d, 1H, J=6 Hz), 2.84 (s, 3H), 2.82 (s, 3H), 2.77-2.81 (m, 2H), 1.84 (t, 2H, J₁=6 Hz), 1.15 (t, 3H, J=6 Hz); ¹³C NMR (900 MHz, MeOD) δ=158.8, 149.4, 147.8, 141.9, 141.0, 140.1, 136.5, 131.6, 126.1, 124.2, 117.0, 115.8, 41.3, 31.8, 29.2, 26.4, 23.0. MS (ESI) 349.7 (M⁺+1).

### 5-(4-hydroxyphenylazo-3-(1-propanone))-2,4-dimethylbenzenesulfonic acid (MS127)

Compound (MS127) was obtained as a yellowish solid (83%). ¹HNMR (Methanol-d₄, 600 MHz) δ=8.37 (s, 1H), 8.11 (s, 1H), 7.93-7.95 (d, 1H, J=12 Hz), 7.13 (s, 1H), 6.94-6.95 (d, 1H, J=6 Hz), 2.95 (q, 2H, J₁=18 Hz, J₂=6 Hz), 2.56 (s, 3H), 2.47 (s, 3H), 1.12 (t, 3H, J=6 Hz); ¹³C NMR (900 MHz, MeOD) δ=164.5, 158.5, 148.2, 146.6, 141.4, 139.1, 138.1, 133.9, 131.1, 123.5, 122.2, 114.6, 114.0, 31.3, 19.1, 15.8, 12.8. MS (ESI) 363.5 (M⁺+1).

### 5-(4-hydroxy-3,5-isopropyl-phenylazo)-2,4-dimethylbenzenesulfonic acid (MS128)

Compound (MS128) was obtained as a yellowish solid (79%). ¹H NMR (Methanol-d₄, 600 MHz) δ=8.37 (s, 1H), 7.81 (s, 2H), 7.39 (s, 1H), 3.50-3.56 (m, 2H), 2.84 (s, 3H), 2.83 (s, 3H), 1.43-1.44 (d, 12H); ¹³C NMR (900 MHz, MeOD) δ=149.4, 147.7, 144.3, 141.2, 140.8, 139.6, 137.9, 136.4, 120.5, 115.8, 36.2, 31.9, 29.1, 26.2. MS (ESI) 391.7 (M⁺+1).

### 5-(4-hydroxy-3-isopropyl-5-methyl-phenylazo)-2,4-dimethylbenzenesulfonic acid (MS129)

Compound (MS129) was obtained as a yellowish solid (83%). ¹H NMR (Methanol-d₄, 600 MHz) δ=8.36 (s, 1H), 7.92 (s, 1H), 7.73 (s, 1H), 7.40 (s, 1H), 3.47 (m, 1H), 2.84 (s, 3H), 2.83 (s, 3H), 2.79 (s, 3H) 1.62 (d, 6H); ¹³C NMR (800 MHz, MeOD) δ=157.3, 148.2, 146.1, 141.5, 139.1, 138.1, 137.3, 133.8, 125.1, 122.4, 120.7, 114.1, 34.4, 28.7, 19.2, 16.0, 15.8. MS (ESI) 377.6 (M⁺+1).

### 5-(4-hydroxy-3-methyl-5-propene-phenylazo)-2,4-methylbenzenesulfonic acid (MS130)

Compound (MS130) was obtained as a yellowish solid (79%). ¹H NMR (Methanol-d₄, 600 MHz) δ=8.34 (s, 1H), 7.74 (s, 1H), 7.72 (s, 1H), 7.40 (s, 1H), 6.17-6.42 (m, 1H), 5.22-5.27 (m, 2H), 3.61-3.62 (d, 2H, J=6 Hz), 2.84 (s, 3H), 2.82 (s, 3H), 2.47 (s, 3H); ¹³C NMR (900 MHz, MeOD) δ=135.7, 128.2, 126.6, 120.9, 119.8, 119.0, 117.1, 115.5, 107.9, 106.3, 104.9, 102.9, 96.6, 94.5, 43.8, 29.4, 26.4, 25.8. MS (ESI) 361.6 (M⁺+1).

### 5-(3-chloro-4-hydroxyphenylazo)-2,4-dimethylbenzenesulfonic acid (MS131)

Compound (MS131) was obtained as a yellowish solid (68%). ¹H NMR (Methanol-d₄, 600 MHz) δ=8.37 (s, 1H), 8.03 (s, 1H), 7.92-7.94 (d, 1H, J=12 Hz), 7.7 (s, 1H), 7.20-7.22 (d, 1H, J=12 Hz), 2.85 (s, 3H), 2.84 (s, 3H); ¹³C NMR (800 MHz, MeOD) δ=155.9, 147.8, 146.6, 141.6, 139.7, 138.8, 134.0, 123.9, 123.3, 121.3, 116.3, 114.1, 19.2, 15.9. MS (ESI) 341.13 (M⁺+1).

### 5-(2,3,5-trimethyl-4-hydroxyphenylazo)-2,4-dimethylbenzenesulfonic acid (MS146)

Compound (MS146) was obtained as a yellowish solid (72%). ¹H NMR (Methanol-d₄, 600 MHz) δ=8.35 (s, 1H), 7.54 (s, 1H), 7.39 (s, 1H), 3.51 (s, 6H), 3.46 (s, 3H), 2.84 (s, 3H), 2.81 (s, 3H); ¹³C NMR (900 MHz, MeOD) δ=151.7, 148.2, 147.7, 144.0, 143.7, 142.8, 141.9, 139.6, 129.1, 128.1, 120.5, 119.4, 29.3, 26.6, 25.8, 22.6, 21.1. MS (ESI) 349.13 (M⁺+1).

### 5-(5-chloro-4-hydroxy-2-methyl-phenylazo)-2,4-dimethylbenzenesulfonic acid (MS154)

Compound (MS154) was obtained as a yellowish solid (77%). ¹H NMR (Methanol-d₄, 600 MHz) δ=7.62 (s, 1H), 7.51 (s, 1H), 7.12 (s, 1H), 6.84 (s, 1H), 2.36 (s, 3H), 2.27 (s, 3H), 2.00 (s, 3H); MS (ESI) 355.04 (M⁺+1).

### Example 3

### Preparation of Compounds of Formula (2)

### Synthesis Scheme

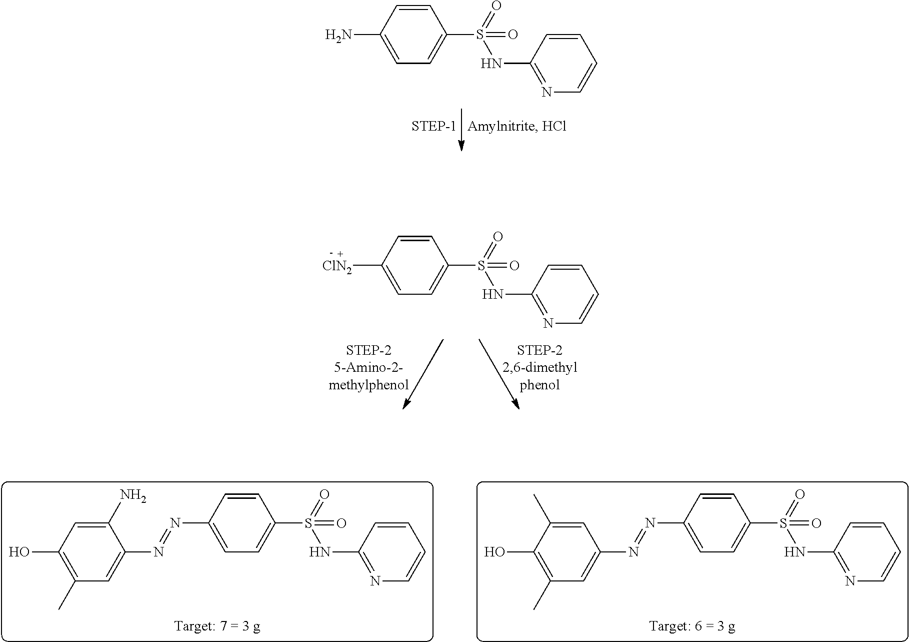

### Experimental Details:

### A. Synthesis of Target-6: MS0129435

To a stirred solution of amine (12 g, 0.04 mol) in methanol and ACN (1:1, 240 mL) was added conc. HCl (20.4 mL) and stirred at 0° C. to −2° C. for 5 min. Then isoamyl nitrite (6.48 mL, 0.055 mol) was added drop wise for 10 min under inert atmosphere and the reaction mixture was stirred at 0° C. Meanwhile a homogenous solution of 1,2-dimethyl phenol (5.84 g, 0.048 mol) and potassium carbonate (33.2 g, 0.24 mol) in water (520 mL) was prepared. This solution was de-gassed by purging with N₂ for 15 min at 0-5° C. and was added via cannula to the previously prepared diazonium salt solution at 0-5° C. and the resulting reaction mixture stirred at 0-5° C. for 1 h. The reaction mixture was then acidified with 1 N HCl (pH=3) and extracted with EtOAc (2×300 mL). The combined organic extracts were dried over Na₂SO₄ and concentrated under reduced pressure to obtain orange solid. This material was purified by column chromatography using 2% MeOH/DCM to afford target-6 (5.6 g, 30.46%).

### TLC: 5% MeOH/DCM, Rf: 0.5)

HPLC purity: 99.17%, IP 10040887
Melting point: 223.5° C.

### Mass: 382 (M+1)

¹HNMR (500 MHz, DMSO-d₆) δ: 12 (bs, 1H), 10.21 (s, 1H), 8.0 (s, 1H), 7.9 (d, 2H), 7.83 (d, 2H), 7.72 (t, 1H), 7.34 (s, 1H), 7.2 (d, 1H), 7.13 (s, 1H), 6.82 (t, 1H), 6.23 (s, 1H), 2 (s, 3H).

### B. Synthesis of Target-7: MS0129436

To a stirred solution of amine (12 g, 0.0481 mol) in methanol and ACN (1:1, 240 mL) was added conc. HCl (20.4 mL) and stirred at 0° C. to −2° C. for 5 min. Then isoamyl nitrite (6.48 mL, 0.553 mol) was added dropwise for 10 min under inert atmosphere and the reaction mixture was stirred at 0° C. for 45 min. Meanwhile homogenous solution of 5-aminocresol (5.92 g, 0.0481 mol) and potassium carbonate (33.2 g, 0.24067 mol) in water (500 mL) was prepared. This solution was de-gassed by purging N₂ for 15 min and then was added via cannula to the previously prepared diazonium salt solution at 0-5° C. and the resulting reaction mixture was stirred at 0-5° C. for 1 h. The reaction mixture was then acidified with 1 N HCl (pH=6) and the reaction was filtered. Fitrate was extracted with EtOAc (2×300 mL) and the solid ppt was stirred in isopropyl alcohol for 3 h at room temperature and filtered. The combined organic extracts were distilled under reduced pressure to obtain orange-red crude residue. The solid was purified by column chromatography (twice) using methanol/DCM to afford target 7 (2.6 g, 14% yield).

### TLC: 5% MeOH/DCM, Rf: 0.5)

HPLC purity: 98.63%, IP 10041325
Melting point: 217.2° C.

### Mass: 383 (M+1)

¹HNMR (500 MHz, DMSO-d₆) δ: 9.22 (bs, 1H), 8.0 (m, 3H), 7.9 (d, 2H), 7.72 (t, 1H), 7.54 (s, 2H), 7.2 (d, 1H), 6.8 (t, 1H), 2.21 (s, 6H).

### C. Synthesis of CM363

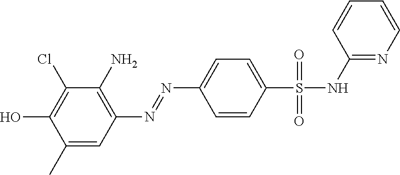 [SMILES: Cc1cc(/N=N/c2ccc(S(=O)(=O)Nc3ccccn3)cc2)c(N)c(Cl)c1O]

### Synthesis of (E)-4-(2-amino-3-chloro-4-hydroxy-5-methylphenyl)diazenyl)-N-(pyridin-2-yl)benzenesulfonamide

A 50 mL round bottom flask was charged with sulfapyridine (100.0 mg, 0.40 mmol, 1.0 eq.) and concentrated HCl (87.5 mg, 160 μL, 2.40 mmol, 5.98 eq.). The mixture was dissolved in a methanol/acetonitrile mixture (3 mL/3 mL). The solution was cooled to 0° C. and stirred for 15 min. Iso-amyl nitrite (47.0 mg, 54 μL, 0.40 mmol, 1.0 eq.) was added drop by drop under argon over 10 min. The solution was stirred at 0° C. for 45 min. Meanwhile, another 50 mL round bottom flask 3-amino-2-chloro-6-cresol (63.0 mg, 0.40 mmol, 1.0 eq.) and potassium carbonate (276.3 mg, 2.0 mmol, 5.0 eq.). To this mixture was added 1.0 mL methanol and 8.0 mL of DI H₂O. The solution was deoxygenated for 15 min. The resultant solution was cooled to 0° C. The previously prepared amber color diazonium ion was added drop wise under argon over 15 min. At the end of the addition, the pH of the solution was maintained between 8-10. The solution was allowed to stir at 0° C. for 1 h and then quenched with 1 N HCl to reach pH 1. Massive precipitation was observed. The product was filtered and dried under vacuum. The pure product appeared as a fine red powder (167.0 mg, 99%). ¹H NMR (DMSO) δ 11.51 (s, 1H), 8.04 (s, 1H), 7.97-7.78 (m, 3H), 7.78-7.62 (m, 3H), 7.53 (s, 1H), 7.15 (s, 1H), 6.89 (s, 1H), 6.73 (br s, 2H), 1.98 (s, 3H). MS calculated for C₁₈H₁₆ClN₅O₃S [M+H]⁺418.08, found 418.08. Purity >99%, t_{R}=5.5 min.

### D. Synthesis of CM267

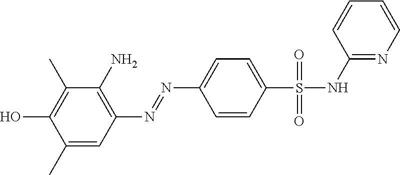 [SMILES: Cc1cc(/N=N/c2ccc(S(=O)(=O)Nc3ccccn3)cc2)c(N)c(C)c1O]

### Synthesis of (E)-4-((2-amino-4-hydroxy-3,5-dimethylphenyl)diazenyl)-N-(pyridin-2-yl)benzenesulfonamide

Following the same procedure as described for CM0000363, the title compound was synthesized. The pure product appeared as a fine brown powder (89%). ¹H NMR (DMSO) δ 8.02 (s, 1H), 7.85 (d, J=7.8, 2H), 7.80-7.61 (m, 3H), 7.39 (s, 1H), 7.15 (d, J=7.8, 1H), 6.88 (s, 1H), 2.01 (s, 3H), 1.88 (s, 3H). MS calculated for C₁₉H₁₉ClN₅O₃S [M+H]³⁰ 398.12, found 398.12. Purity >99%, t_{R}=5.4 min.

### E. Synthesis of C11298

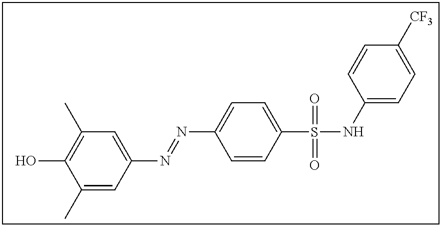 [SMILES: Cc1cc(/N=N/c2ccc(S(=O)(=O)Nc3ccc(C(F)(F)F)cc3)cc2)cc(C)c1O]

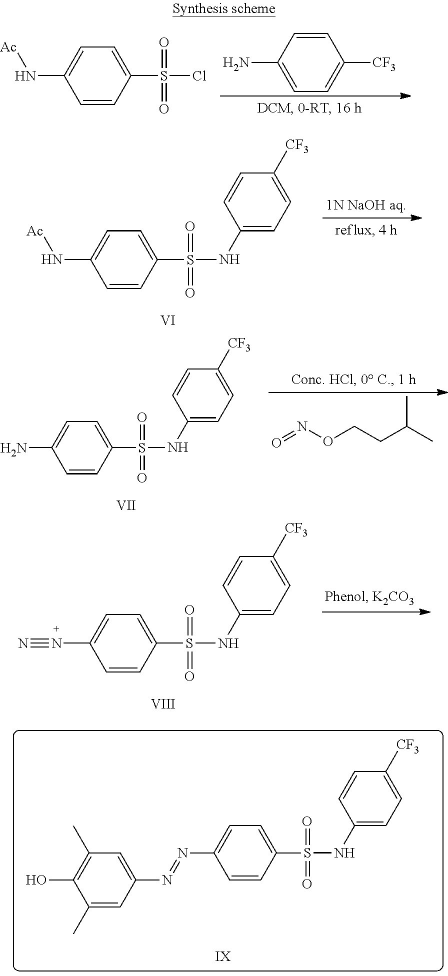

### Synthetic procedure for: (E)-4-(4-hydroxy-3,5-dimethylphenyl)diazenyl)-N-(4-(trifluoromethyl)phenyl)benzenesulfonamide (IX)

### N-(4-(N-(4-(trifluoromethyl)phenyl)sulfamoyl)phenyl)acetamide (VI)

The title compound appeared as a yellow powder. ¹H NMR (DMSO) δ 10.79 (s, 1H), 10.34 (s, 1H), 7.78-7.71 (m, 4H), 7.59 (d, J=8.4, 2H), 7.26 (d, J=8.4, 2H), 2.09 (s, 3H). MS calculated for C₁₅H₁₃F₃N₂O₃S [M+H]⁺359.07, found 359.07. Purity >99%, t_{R}=5.9 min.

### 4-amino-N-(4-(trifluoromethyl)phenyl)benzenesulfonamide (VII)

The procedure is exactly the same as describe for II. ¹H NMR (CDCl₃) δ 7.62 (d, J=8.4, 2H), 7.50 (d, J=8.4, 2H), 7.18 (d, J=8.4, 2H), 7.08 (s, 1H), 6.63 (d, J=8.4, 2H). MS calculated for C₁₅H₁₃F₃N₂O₃S [M+H]⁺ 317.06, found 317.08. Purity >95%, t_{R}=5.9 min.

### (E)-4-((4-hydroxy-3,5-dimethylphenyl)diazenyl)-N-(4-(trifluoromethyl)phenyl)benzenesulfonamide (CM298)

Following the same procedure as described in CM363, instead of the almost instantaneous precipitation, the “product” oiled out. After adjusting the pH to 1, the product oiled out. The resultant solution was extracted with diethyl ether (10 mL×3). The combined organic layer was washed with brine and dried over magnesium sulfate. Purification by automatic chromatography (40:60 ethyl acetate in hexane, R_{f}=0.49, 105.0 mg, 75%) provided the target molecule as a bright orange powder. ¹H NMR (DMSO) δ 11.02 (s, 1H), 9.38 (s, 1H), 7.98 (d, J=8.4, 2H), 7.63 (d, J=8.4, 2H), 7.59 (s, 2H), 7.31 (d, J=8.4, 2H), 2.26 (s, 6H). MS calculated for C₂₁H₁₈F₃N₃O₃S [M+H]⁺ 450.10, found 450.10. Purity >95%, t_{R}=6.5 min.

### F. Synthesis of CM280

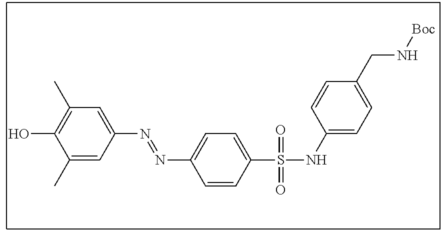 [SMILES: Cc1cc(/N=N/c2ccc(S(=O)(=O)Nc3ccc(CNC(=O)OC(C)(C)C)cc3)cc2)cc(C)c1O]

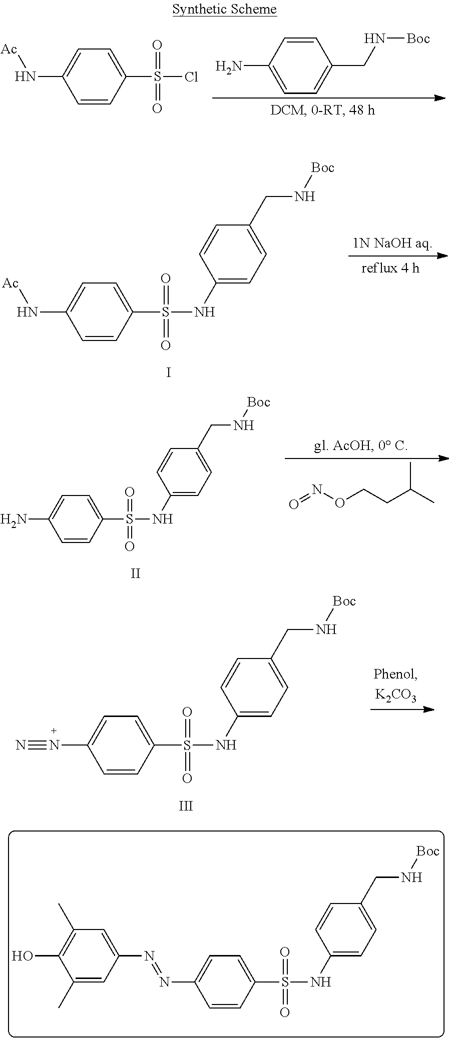

tert-butyl 4-(4-acetamidophenylsulfonamido)benzylcarbamate (1). A 100 mL round bottom flask was charged with N-Acetylsulfanilyl chloride (525.0 mg, 2.25 mmol, 1.0 eq.) and was dissolved in anhydrous pyridine (30 mL). After cooling to 0° C. in an ice bath, the solution was allowed to stir vigorously at the same temperature for 10 min. 4-(N-Boc)aminomethyl aniline (500.0 mg, 2.25 mmol, 1.0 eq.) was dissolved in pyridine (20 mL) and added carefully drop wise over 15 min. 1 h after the addition was complete, the solution was gradually warmed up to rt. The mixture was stirred at rt overnight. The pyridine was removed under reduced pressure by forming an azeotrope with toluene. Purification by automatic chromatography (1:20 methanol in dichloromethane, R_{f}=0.22, 542.0 mg, 58%) provided the title compound as a beautiful pink crystal. ¹H NMR (CDCl₃) δ 8.95 (s, 1H), 7.71 (t, 1H), 7.59 (d, J=8.4, 2H), 7.54 (d, J=8.4, 2H), 7.31 (m, 2H), 7.04 (m, 2H), 5.23 (s, 1H), 4.19 (s, 2H), 2.12 (s, 3H), 1.44 (s, 9H). MS calculated for C₂₀H₂₅N₃O₅S [M+Na]⁺ 442.14, found 442.14. Purity >99%, t_{R}=5.7 min.

### tert-butyl 4-(4-aminophenylsulfonamido)benzylcabamate (II)

A 200 mL round bottom flask was charged with compound I (542.0 mg, 1.29 mmol, 1.0 eq.) and ethanol (28.0 mL). To this solution was added NaOH aqueous solution (3N, 14 mL, 25.6 eq.). The solution was allowed to heat up to 100° C. and reflux for 7 h. The organic solvents were removed in vacuo. The pH of the aqueous solution was carefully neutralized to pH3 with 1.0 M HCl. At that time, large amount of cotton-like precipitate was observed. The resultant aqueous layer was extracted with ethyl acetate (20 mL×4). The combined organic layer was dried on sodium sulfate. After concentrated in vacuo, the residual was stored at 4° C. overnight. The pure product appeared as a beautiful yellow crystal (500 mg, 100%). ¹H NMR (DMSO) δ 9.80 (s, 1H), 7.37 (d, J=8.4, 2H), 7.05 (d, J=8.4, 2H), 6.99 (d, J=8.4, 2H), 6.52 (d, J=8.4, 2H), 4.00 (d, J=6.0, 2H), 1.37 (s, 9H). MS calculated for C₁₈H₂₃N₃O₄S [M+H]⁺ 400.14, found 400.14. Purity >99%, t_{R}=5.7 min.

### (E)-tert-butyl 4-(4-((4-hydroxy-3,5-dimethylphenyl)diazenyl)phenylsulfonamido)benzylcarbamate (CM280)

A 50 mL round bottom flask was charged with compound II (106.9 mg, 0.29 mmol, 1.0 eq.) and glacial acetic acid (1.37 g, 1.30 mL, 22.7 mmol, 22.0 eq.). The mixture was dissolved in a methanol/acetonitrile mixture (3 mL/3 mL). The reaction solution was cooled to 0° C. and stirred for 15 min. tert-butyl nitrite (2.08 g, 2.39 mL, 20.2 mmol, 19.5 eq.) was added drop by drop under argon over 10 min. The yellow solution was stirred at 0° C. for 45 min. Meanwhile, 2,6-dimethylphenol (125.0 mg, 1.02 mmol, 1.0 eq.) and potassium carbonate (707.1 mg, 5.1 mmol, 5.0 eq.) were mixed in a separate 50 mL round bottom flask and dissolved in methanol (1.5 mL). To this solution was added DI H₂O (8.0 mL). The resultant solution was degassed with argon for 15 min before it was cooled to 0° C. The previously prepared amber color diazonium ion (III) was added drop wise under argon over 15 min. At the end of the addition, the pH of the solution was maintained between 8-10. The solution was allowed to stir at 0° C. for 1 h and then rt overnight. At the end of the reaction, the pH of the solution was carefully adjusted to pH 3 using 1 M HCl. The resultant mixture was extracted with diethyl ether (10 mL×3). The organic layer was washed with brine and then dried over sodium sulfate. The volatiles were removed in vacuo. Purification by automatic chromatography (3:2 ethyl acetate in hexane, R_{f}=0.36, 45.0 mg, 30%) provided the title compound as orange oil. ¹H NMR (CDCl₃) δ 9.70 (br s, 1H), 7.76 (d, J=7.8, 2H), 7.42 (d, J=6.6, 2H), 7.29 (s, 2H), 7.17 (d, J=7.8, 2H), 7.06 (d, J=6.6, 2H), 4.88 (br s, 1H), 4.26 (d, J=4.2, 2H), 2.05 (s, 6H), 1.46 (s, 9H). MS calculated for C₂₆H₃₀N₄O₅S [M+Na]⁺ 533.18, found 533.18. Purity >99%, t_{R}=6.4 min.

### Example 4

### Preparation of Compounds of Formula (2)

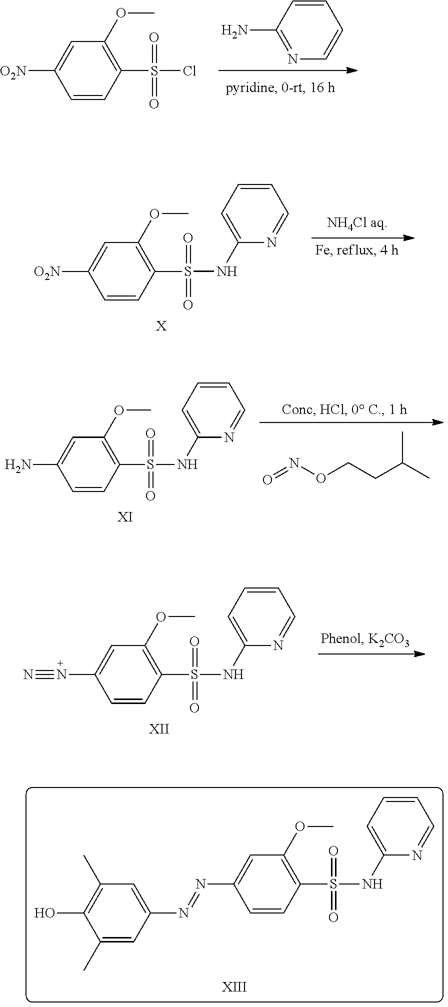

### A. Synthesis of (E)-4-(4-hydroxy-3,5-dimethylphenyl)diazenyl)-2-methoxy-N-(pyridin-2-yl)benzenesulfonamide (XIII)

### 2-methoxy-4-nitro-N-(pyridin-2-yl)benzenesulfonamide (X)

A 4 mL scintillation vial was charged with 4-nitrobenzenesulfonyl chloride (50.0 mg, 0.20 mmol, 1.0 eq.), 2-aminopyridine (18.7 mg, 0.20 mmol, 1.0 eq.), and pyridine (0.5 mL). The solution was allowed to stir vigorously at 0° C. for 10 min. 1 h after the addition was complete, the solution was gradually warmed up to rt. As the reactions progressed, the solution turned dirt yellow and a lot of precipitate was observed. The solvent pyridine was removed under reduced pressure by forming an azeotrope with toluene. Purification by automatic chromatography (5:95 methanol in dichloromethane, R_{f}=0.70) provided the target molecule as a light yellow crystal (40.0 mg, 65%). ¹H NMR (DMSO) δ 8.11 (d, J=8.4, 2H), 8.00-7.90 (m, 3H), 7.85 (s, 1H), 7.80 (m, 1H), 7.30 (br s, 1H), 6.87 (m, 1H), 3.83 (s, 3H). MS calculated for C₁₂H₁₁N₃O₅S [M+H]⁺ 310.05, found 310.08. Purity >99%, t_{R}=5.2 min.

### B. Synthesis of CM280

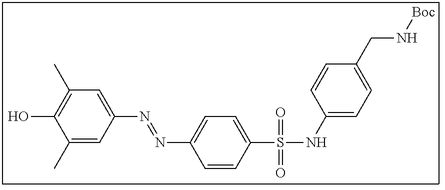 [SMILES: Cc1cc(/N=N/c2ccc(S(=O)(=O)Nc3ccc(CNC(=O)OC(C)(C)C)cc3)cc2)cc(C)c1O]

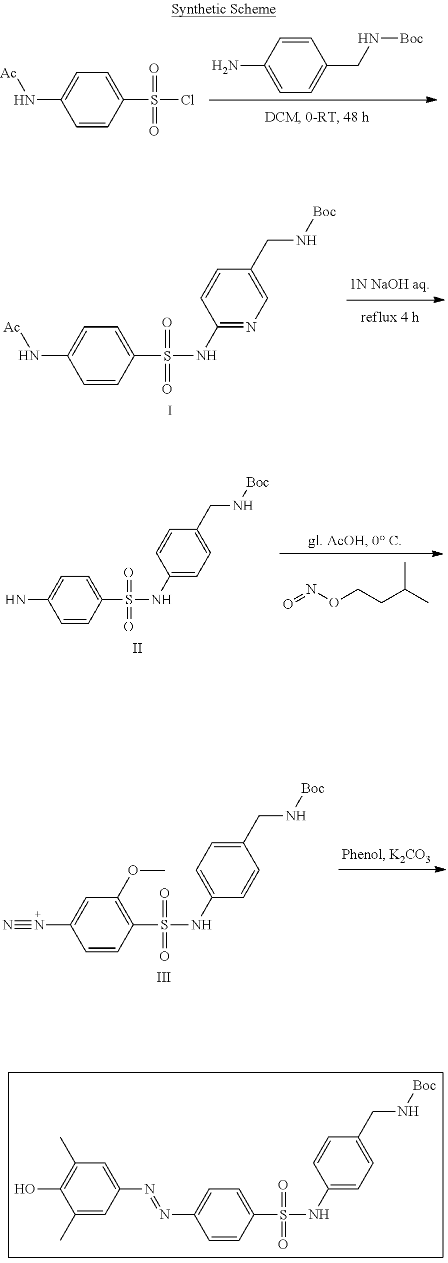

### tert-butyl 4-(4-acetamidophenylsulfonamido)benzylcarbamate (I)

A 100 mL round bottom flask was charged with N-acetylsulfanilyl chloride (525.0 mg, 2.25 mmol, 1.0 eq.) and was dissolved in anhydrous pyridine (30 mL). After cooling to 0° C. in an ice bath, the solution was allowed to stir vigorously at the same temperature for 10 min. 4-(N-Boc)aminomethyl aniline (500.0 mg, 2.25 mmol, 1.0 eq.) was dissolved in pyridine (20 mL) and added carefully drop wise over 15 min. 1 h after the addition was complete, the solution was gradually warmed up to rt. The mixture was stirred at rt overnight. The pyridine was removed under reduced pressure by forming an azeotrope with toluene. Purification by automatic chromatography (1:20 methanol in dichloromethane, R_{f}=0.22, 542.0 mg, 58%) provided the title compound as a beautiful pink crystal. ¹H NMR (CDCl₃) δ 8.95 (s, 1H), 7.71 (t, 1H), 7.59 (d, J=8.4, 2H), 7.54 (d, J=8.4, 2H), 7.31 (m, 2H), 7.04 (m, 2H), 5.23 (s, 1H), 4.19 (s, 2H), 2.12 (s, 3H), 1.44 (s, 9H). MS calculated for C₂₀H₂₅N₃O₃S [M+Na]⁺ 442.14, found 442.14. Purity >99%, t_{R}=5.7 min.

### tert-butyl 4-(4-aminophenylsulfonamido)benzylcarbamate (II)

A 200 mL round bottom flask was charged with compound I (542.0 mg, 1.29 mmol, 1.0 eq.) and ethanol (28.0 mL). To this solution was added NaOH aqueous solution (3N, 14 mL, 25.6 eq.). The solution was allowed to heat up to 100° C. and reflux for 7 h. The organic solvents were removed in vacuo. The pH of the aqueous solution was carefully neutralized to pH3 with 1.0 M HCl. At that time, large amount of cotton-like precipitate was observed. The resultant aqueous layer was extracted with ethyl acetate (20 mL×4). The combined organic layer was dried on sodium sulfate. After concentrated in vacuo, the residual was stored at 4° C. overnight. The pure product appeared as a beautiful yellow crystal (500 mg, 100%). ¹H NMR (DMSO) δ 9.80 (s, 1H), 7.37 (d, J=8.4, 2H), 7.05 (d, J=8.4, 2H), 6.99 (d, J=8.4, 2H), 6.52 (d, J=8.4, 2H), 4.00 (d, J=6.0, 2H), 1.37 (s, 9H). MS calculated for C₁₈H₂₃N₃O₄S [M+H]⁺ 400.14, found 400.14. Purity >99%, t_{R}=5.7 min.

### (E)-tert-butyl 4-(4-((4-hydroxy-3,5-dimethylphenyl)diazenyl)phenylsulfonamido)benzylcarbamate (CM280)

A 50 mL round bottom flask was charged with compound II (106.9 mg, 0.29 mmol, 1.0 eq.) and glacial acetic acid (1.37 g, 1.30 mL, 22.7 mmol, 22.0 eq.). The mixture was dissolved in a methanol/acetonitrile mixture (3 mL/3 mL). The reaction solution was cooled to 0° C. and stirred for 15 min. tert-butyl nitrite (2.08 g, 2.39 mL, 20.2 mmol, 19.5 eq.) was added drop by drop under argon over 10 min. The yellow solution was stirred at 0° C. for 45 min. Meanwhile, 2,6-dimethylphenol (125.0 mg, 1.02 mmol, 1.0 eq.) and potassium carbonate (707.1 mg, 5.1 mmol, 5.0 eq.) were mixed in a separate 50 mL round bottom flask and dissolved in methanol (1.5 mL). To this solution was added DI H₂O (8.0 mL). The resultant solution was degassed with argon for 15 min before it was cooled to 0° C. The previously prepared amber color diazonium ion (III) was added drop wise under argon over 15 min. At the end of the addition, the pH of the solution was maintained between 8-10. The solution was allowed to stir at 0° C. for 1 h and then rt overnight. At the end of the reaction, the pH of the solution was carefully adjusted to pH 3 using 1 M HCl. The resultant mixture was extracted with diethyl ether (10 mL×3). The organic layer was washed with brine and then dried over sodium sulfate. The volatiles were removed in vacuo. Purification by automatic chromatography (3:2 ethyl acetate in hexane, R_{f}=0.36, 45.0 mg, 30%) provided the title compound as orange oil. NMR (CDCl₃) δ 9.70 (br s, 1H), 7.76 (d, J=7.8, 2H), 7.42 (d, J=6.6, 2H), 7.29 (s, 2H), 7.17 (d, J=7.8, 2H), 7.06 (d, J=6.6, 2H), 4.88 (br s, 1H), 4.26 (d, J=4.2, 2H), 2.05 (s, 6H), 1.46 (s, 9H). MS calculated for C₂₆H₃₀N₄O₅S [M+Na]⁺ 533.18, found 533.18. Purity >99%, t_{R}=6.4 min.

### Example 5

### Inhibition of p53 Activiation Upon DNA Damaging Stress

### 5-(2-amino-4-hydroxy-5-methylphenylazo)-2,4-dimethylbenzenesulfonic acid (Ischemin)

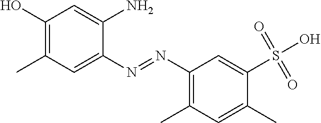 [SMILES: Cc1cc(/N=N/c2cc(S(=O)(=O)O)c(C)cc2C)c(N)cc1O]

### Cell Lines, Plasmids and Reagents

U20S cells were grown in DMEM (Eagle's minimal essential medium) (Mediatech) supplemented with 10% fetal bovine serum (Invitrogen) and antibiotics (Invitrogen). For p53 activation, doxorubicin (Sigma) was used. The compounds were dissolved in DMSO (Sigma). The antibodies used for immunoprecipitation and western blot are p53 (sc-6243), p21 (sc-397), 14-3-3 (sc-7683), lamin B (sc-6215) from Santa Cruz Biotech; p53Ser15p (9282), p53K382ac (2525), ATM (2873), ATMp1981 (4526), CHK (2345), CHKp (2341) and PUMA (4976) from Cell Signaling Tech; H3 (ab1791), H3KS10p (ab14955), H3K9ac (ab4441) from ABCAM; and Actin A4700) from Sigma.

### Western Blotting

U20S cells were harvested cells and lysed in lysis buffer (20 mM Tris (pH 8.0), 150 mM NaCl, 1 mM EGTA, 1% Triton X-100, and 50 mM NaF) containing protease inhibitor cocktail (Sigma). The cells were sonicated and spun down at 14,000 rpm for 30 min at 4° C. After protein estimation, 30-50 micrograms of lysates were subjected to SDS-PAGE, transferred onto nitrocellulose membranes, blocked with 5% milk/PBS and blotted with a primary antibody. Horse radish peroxidase-labeled secondary antibodies (goat anti-Mouse or anti-Rabbit) were added for 60 min at room temperature, and the blots were washed with TBS (20 mM Tris, 150 mM Nacl, and 0.05% tween −20) and subjected to autoradiography after development of reaction by ECL (GE health care).

### Luciferase Assay

U20S Cells were transfected with p21 luciferase (1 μg) and renilla luciferase (100 ng) vectors in 6 well plate format using Fugene 6 (Roche). Briefly, total of 1.1 micrograms of vector was incubated with 3 mL of Fugene 6 reagent for 30 min. After 3-4 hours of transfection, cell were treated with compounds for overnight, and then exposed to 300 nanogram of doxorubicin for next 24 hours. In these experiments, DMSO, transfected cells with empty vector and cell without doxorubicin were used as controls. DMSO concentration is maintained at 0.01%. Transfected cells with doxorubicin treatment were used as positive control. The luciferase activity was estimated by following the manufacturer's instruction (Promega) in a luminometer. Both active and passive lysis of cells yielded consistent results. The inhibitory activity (IC₅₀) of a small molecule on p21 luciferase activity was obtained from the average of three biological replicates using PRISM software.

### BRDU Cell Cycle Analysis

BRDU incorporation assay for cell cycle evaluation was performed in 96 well plates using calorimetric based kit from Calbiochem (Cat# QiA58). Hundred microliter of 1×10⁵/ml cells were plated in DMEM media (Mediatech) with 10% fetal bovine serum (FBS). After 12 hours cells were treated with compounds ischemin and MS 119 (50 μM) with or without doxorubicin treatment (5 μM). The controls were DMSO and untreated cells. BRDU was added for 24 hours treatment. After 24 hours cells were fixed and treated with anti-BRDU antibody. After washings, the wells were incubated with peroxidase. After final wash, the color was developed using TMB as substrate and the reaction was stopped with stop solution and optical density was estimated at 450 nm.

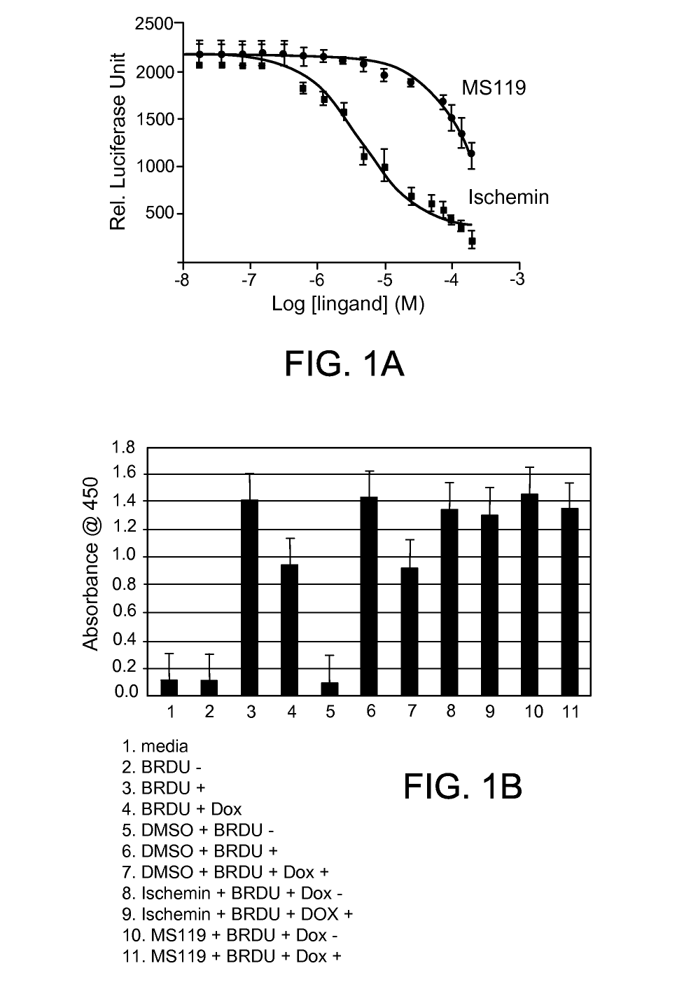

DNA damage induced by doxorubicin leads to p53 stimulated cellular responses including cell cycle arrest, damage repair, and apoptosis. To determine the effect of ischemin on dividing U20S cells, U20S cells were treated with 5-bromo-2-deoxyuridine (BRDU) and the incorporated BRDU during in DNA synthesis was measured using an ELISA assay. The result showed that doxorubicin treatment of U20S cells resulted in a 45% decrease of BRDU incorporation, indicative of doxorubicin induced cell cycle arrest. However, the presence of ischemin or MS 119 (50 μM) almost completely prevented U20S cells from undergoing doxorubicin-induced cell cycle arrest (FIG. 1). Note that these results also indicate that ischemin is not toxic to the cells at this concentration.

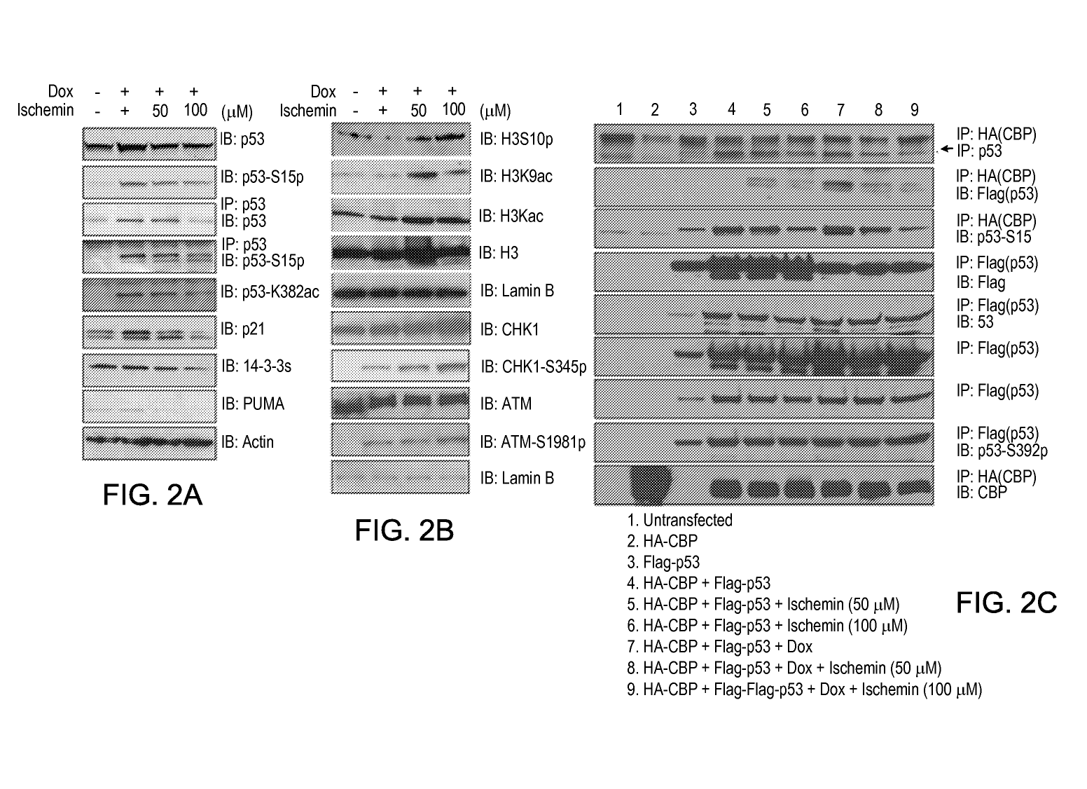

The biochemical effects of ischemin on p53 stability and function as transcription factor was examined. U20S cells were incubated in the presence of doxorubicin with or without ischemin at concentration of 50 or 100 μM for 24 hours. Subsequently, cellular proteins were subjected to western blot analysis (as described above). As shown in FIG. 2A, the doxorubicin-induced increased levels of p53 protein, its Ser15-phosphorylated (p53S15p) and Lys382-acetylated (p53K382ac) forms underwent marked reduction in the presence of ischemin as assessed by direct western blots of cell lysate or following immunoprecipitation. Further, it was observed that p53 directed expression of its target genes p21, PUMA and 14-3-3s induced by doxorubicin retreatment was significantly decreased in the presence of ischemin whereas the level of actin remained the same.

### HA-CBP and Flag-p53 Pull-Down Assay

HA-CBP and Flag-p53 were transfected into human embryonic kidney (HEK) 293T cells with recommended amount of Fugene 6 (Roche). After transfection, the HA-CBP and Flag-p53 co-transfected cells were treated with ischemin in the presence or absence of doxorubicin. To test the inhibitory potential of ischemin against CBP and p53 association, CBP was first immuno-precipitated by pulling-down with HA-agarose beads (Sigma) and its association with p53 was then determined with western blot using anti-Flag antibody (Sigma).

As a transcription factor, p53 ability to activate gene expression is also dependent upon chromatin modifications. Since CBP acetylates both histones and p53, the possible changes of epigenetic marks on p53 and global histones in presence of ischemin was evaluated. The western blot analysis of the nuclear extracts from U20S cells revealed that p53 inhibition by ischemin is associated with an increase in histone H3 phosphorylation at Ser10 and a decrease in H3 acetylation at Lys9 (FIG. 2B). These changes of post-translational modifications on p53 and histone H3 are associated with down-regulation of p21, PUMA and 14-3-3, but not the controls of actin, histone H3 and lamin B. In addition, ischemin treatment did not affect the level or functional phosphorylation state of ATM and CHK1, which are the upstream signal transducers of p53 (FIG. 2B). Collectively, these results suggest that ischemin inhibits doxorubicin-induced p53 activation and transcriptional functions by altering post-translational modification states on p53 and histones.

It was also investigated whether ischemin down-regulates p53 by blocking p53 binding to CBP. Haemaglutinin-tagged CBP(HA-CBP) and Flag-tagged p53 (Flag-p53) was overexpressed in human embryonic kidney (HEK) 293T cells. Treatment of the 293T cells with ischemin in the presence or absence of doxorubicin did not affect the expression of HA-CBP or Flag-p53, or acetylation and phosphorylation levels on p53 as assessed by immunoprecipitation with anti-Flag antibody followed by Western blot analysis using specific antibodies (FIG. 2C). The results reveal that ischemin was capable of inhibiting in a dose-dependent manner p53 binding to CBP, particularly upon under doxorubicin treatment (FIG. 2C, lanes 8 and 9 vs. lane 7). Note that p53 associated with HA-CBP is phosphorylated on Ser15, indicating that p53 is transcriptionally active. These results confirm that ischemin inhibits p53-induced p21 activation upon doxorubicin exposure by blocking p53 recruitment of CBP, which is required for p53 target gene activation.

### Example 6

### Inhibition of p53 Cellular Signaling Pathways

### Microarray Analysis

The selectivity of ischemin in transcription inhibition of p53 target genes was evaluated using a RT-PCR array analysis of RNA isolated from biological samples of U20S cells. The array was performed on RNA isolated from three different biological repeats in U20S cells using a set of primers selected for a group of genes that are known to be associated within p53 signaling pathways. The differentially expressed genes in treated related to untreated groups, i.e. doxorubicin treated versus untreated, or doxorubicin plus ischemin versus doxorubicin alone, were subjected to pathway analysis by using the Ingenuity System software. The fold changes of these genes were converted to log2Ratio and then imported into IPA tool along with gene symbols. The enriched pathways in the gene list were identified by Fisher exact test at p value of 0.05 and visualized in Canonical pathway explorer.

The results show that doxorubicin treatment up-regulated p53 target genes that include CCNB2, CCNH, CDC25C, and CDK4, but did not affect housekeeping genes GAPDH, 13-2 microglobulin (B2M) and actin (ACTB). On the other hand, ischemin can differentially reduce doxorubicin-induced expression of p53 target genes CCNE2, CCNG2, CDC2, CDC25A, CDKN1A, CDKN2A (p21), GADD45A, E2F1, E2F3, PCNA, SESN1 and SESN2. These gene products are known to participate in different cellular pathways driven by p53, of which the best known is CDKN1A (p21) that functions as an inhibitor for cell cycle progression. Taken together, these results confirm our hypothesis that small-molecule inhibition of the acetyl-lysine binding activity of the CBP BRD could down-regulate p53 activation and its ability to activate its target genes under stress conditions.

### Example 7

### Cellular Protective Agent Against Myocardial Ischemic Stress

The ability of ischemin to inhibit apoptosis in cardiomyocytes under DNA damage stress was evaluated. Primary neonatal rat cardiomyocytes were isolated and maintained in culture, then, treated with doxorubicin for 24 hours to induce DNA damage in the presence or absence of ischemin. The DNA damage induced by apoptosis was analyzed by the TUNEL (terminal deoxynucleotidyl transferase dUTP nick and end labeling) assay, in which a terminal deoxynucleotidyl transferase was used to identify 3′-OH of DNA generated by DNA fragmentation resulting from apoptosis, and then labels it with biotinylated dUTP. The latter was then detected with avidin-conjugated FITC for specific staining.

### Cardiac Myocyte Isolation

Neonatal rat ventricular myocytes (NRVMs) were isolated by enzymatic dissociation of cardiac ventricle from 1-to-2-day-old Sprague-Dawley pups using the Worthington neonatal cardiomyocyte isolation system (Worthington). Briefly, the pups were anesthetized and their hearts were excised. The ventricular tissues were minced in ice cold HBSS and then digested with trypsin overnight at 4° C. followed by collagenase treatment for 45 min at 37° C. Cells were collected by centrifugation at 800 rpm for 5 min and subsequently underwent two rounds of preplating on culture dishes to minimize nonmyocyte contamination. The enriched cardiomyocytes were cultured in DMEM/F12 nutrient mixture (Invitrogen) with 10% horse serum and 5% fetal calf serum (Invitrogen). After 48 hours, the medium was changed to DMEM/F12 containing 1% insulin, transferrin, and selenium media supplement (ITS; Invitrogen) and 0.1% BSA.

Apoptosis Assays in Cardiomyocytes

Caspase 3/7 and TUNEL assays were performed to assess inhibition of apoptosis by ischemin. Caspase assay and TUNEL assays were performed using Caspase-Glo 3/7 and DeadEnd kits from Promega. Caspase assay was performed on live cardiomyocytes in 96 wells plate on three different days. Similarly, TUNEL assay was performed in triplicate on three different days. For caspase assay 7500 cardiomyocytes were plated in 96 well plates. After treatment with compounds overnight and then doxorubicin for 24 hours, the intensities of luminecnce were read. Similarly, the TUNEL assay was performed on cardiomyocytes attached on coverslips. Briefly, cells were fixed with 4% paraformaldehyde in phosphate buffer saline and permeablized with 0.5% Tween 20 for 10 minutes. The TUNEL reaction was performed on cells with nucleotide labeled with FITC by following manufacturer's instruction.

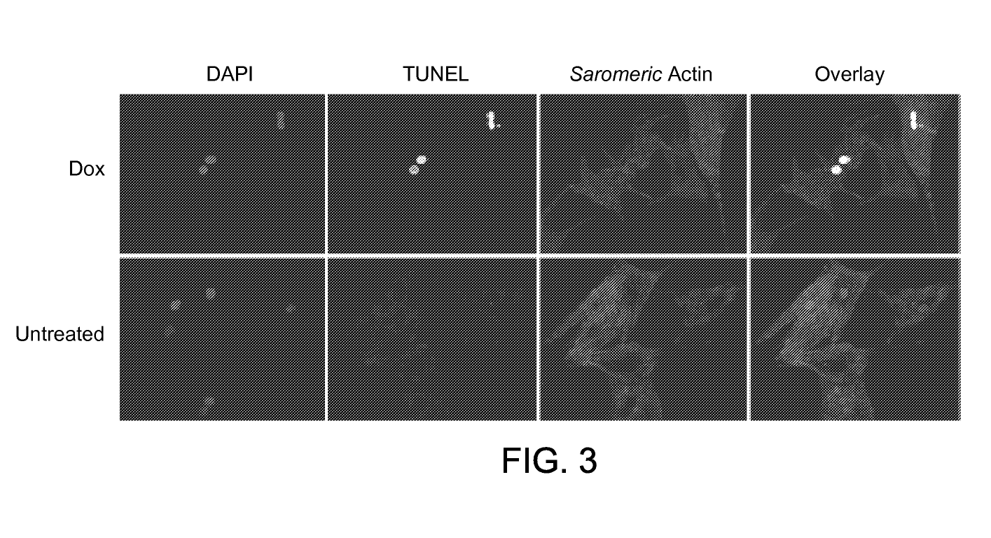

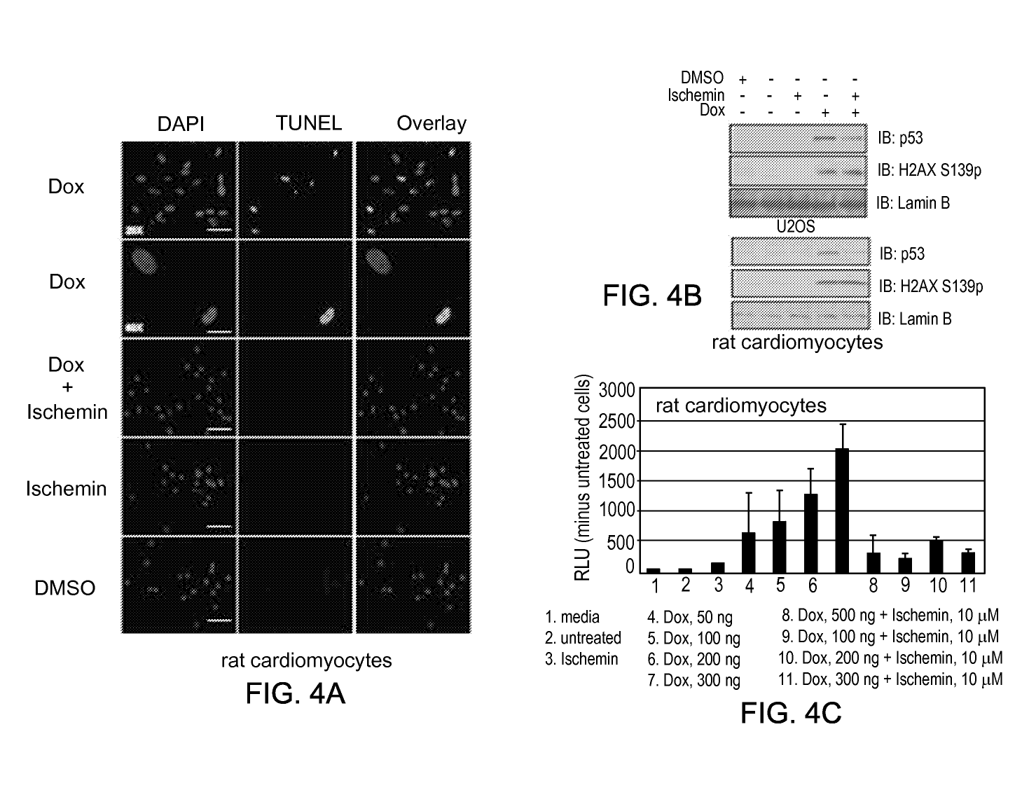

Using this TUNEL assay it was observed that doxorubicin treatment induces apoptosis in the cardiomyocytes (FIG. 3), and observed that ischemin, which has no toxicity of its own, can effectively inhibit doxorubicin-induced apoptosis in the cardiomyocytes (FIG. 4A). Further, similar to U20S cells, it was confirmed that ischemin was able to inhibit doxorubicin induced p53 activation in the primary neonatal rat cardiomycocytes, but did not alter H2AX phosphorylation at Ser139 (FIG. 4B). The latter argues that ATM is active in presence of ischemin, which is consistent with our analysis using Western blots (FIG. 2B). Ischemin likely blocks apoptosis in cardiomyocytes by inhibiting caspase 3/7 activity in a dose-dependent manner (FIG. 4C). Finally, it was ruled out that ischemin's ability to directly inhibit the lysine acetyltransferase activity of CBP/p300 towards a histone H3 peptide substrate in a fluorescence-based assay (data not shown). Taken together, these results demonstrate that ischemin is cell permeable and capable of functioning as a cellular protective agent against myocardial damage by down-regulating p53-induced apoptosis under the stress conditions.

### Example 8

### Inhibition of Gene Transcriptional Activity of NF-kB in Inflammation by BRD Inhibitors

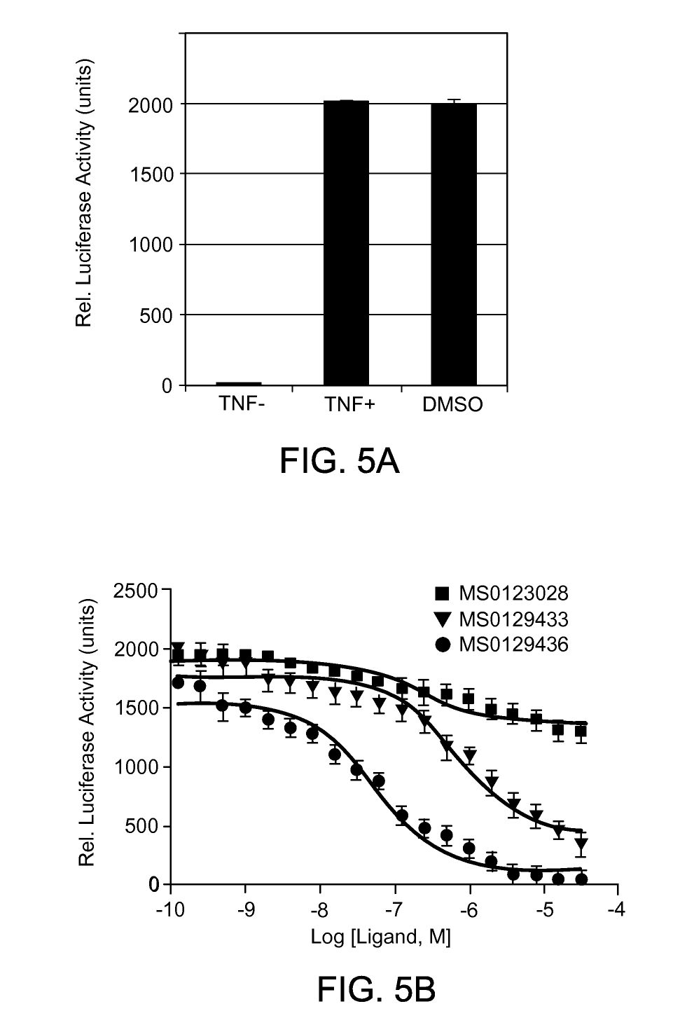

Dysregulation of macrophages and T cell functions trigger inflammatory responses contributing to IBD progression. Given its pro-inflammatory functions, NF-κB inhibition has anti-inflammatory effects, as shown by inhibition of IKK activity, which prevents phoshorylation and release of IκBα from NF-κB. Our study shows that bromodomain inhibitors can inhibit NF-κB pro-inflammatory functions by blocking its acetylation by p300/CBP or PCAF, or its acetylation-mediated recruitment of transcriptional cofactor BRD4 required for target gene activation. As shown in FIG. 5 and Table 5, it was observed that treatment of NF-κB-response element stabilized HEK293 cells with a BRD inhibitor MS0123028, identified as a HTS hit, results in inhibition of TNFα-induced activation of NF-κB in a dose-dependent manner (IC₅₀=220 nM), and the inhibition is more profound with our newly developed compounds MS0129433 and MS0129436 (IC50=57 nM) (related to compounds of formula (1) and (2) that bind to bromomdomains of p300/CBP and BRD4 with higher affinity. These results support the notion that inhibition of lysine-acetylated NF-κB binding to transcriptional co-activators or cofactors with small molecule bromomdomain inhibitors represents a novel mechanism that can modulate NF-κB proinflammatory activity in cells.

TABLE 5

| | | PCAF | CBP | BrD4-1 | BrD4-2 |
|---|---|---|---|---|---|
| Structure | Compound | (μM) | (μM) | (μM) | (μM) |
| 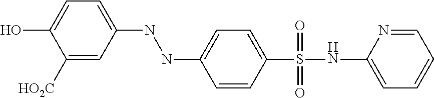 [SMILES: O=C(O)c1cc(NNc2ccc(S(=O)(=O)Nc3ccccn3)cc2)ccc1O] | Sulfasalazine (MS0123028) | 88 | 82 | 54 | 54 |
| 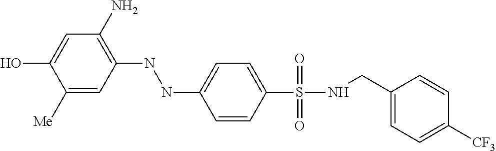 [SMILES: Cc1cc(NNc2ccc(S(=O)(=O)NCc3ccc(C(F)(F)F)cc3)cc2)c(N)cc1O] | MS0129433 | >100 | >100 | 4.5 | 21.0 |
| 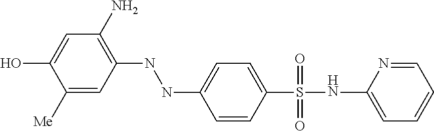 [SMILES: Cc1cc(NNc2ccc(S(=O)(=O)Nc3ccccn3)cc2)c(N)cc1O] | MS0129436 | 8.6 | 14.0 | 6.0 | 5.7 |

### Example 9

### Molecular Basis of Lead Recognition by the CBP BRD

To understand the molecular basis of CBP BRD recognition of the diazobenzenes, the three-dimensional structure of the ischemin/CBP BRD complex was determined by using NMR. NMR samples contained a protein/ligand complex of ˜0.5 mM in 100 mM phosphate buffer, pH 6.5 that contains 5 mM perdeuterated DTT and 0.5 mM EDTA in H₂O/²H₂O (9/1) or ²H₂O. All NMR spectra were collected at 30° C. on NMR spectrometers of 800, 600 or 500 MHz. The ¹H, ¹³C and ¹⁵N resonances of a protein of the complex were assigned by triple-resonance NMR spectra collected with a ¹³C/¹⁵N-labeled and 75% deuterated protein bound to an unlabeled ligand (Clore and Gronenborn, 1994). The distance restraints were obtained in 3D ¹³C- or ¹⁵N-NOESY spectra. Slowly exchanging amides, identified in 2D ¹⁵N-HSQC spectra recorded after a H₂O buffer was changed to a ²H₂O buffer, were used with structures calculated with only NOE distance restraints to generate hydrogen-bond restraints for final structure calculations. The intermolecular NOEs were detected in ¹³C-edited (F₁), ¹³C/¹⁵N-filtered (F₃) 3D NOESY spectrum. Protein structures were calculated with a distance geometry-simulated annealing protocol with X-PLOR (Brunger, 1993). Initial structure calculations were performed with manually assigned NOE-derived distance restraints. Hydrogen-bond distance restraints, generated from the H/D exchange data, were added at a later stage of structure calculations for residues with characteristic NOEs. The converged structures were used for iterative automated NOE assignment by ARIA for refinement (Nilges and O'Donoghue, 1998). Structure quality was assessed by Procheck-NMR (Laskowski et al., 1996). The structure of the protein/ligand complex was determined using intermolecular NOE-derived distance restraints.

The overall position and orientation of ischemin bound to CBP BRD is similar to that of the initial hit MS456. It is worth noting that binding ischemin caused severe line broadening of several protein residues at the ligand-binding site, which include Pro1110, Phe1111, Ile1122, Tyr1125, Ile1128, and Tyr1167. The ligand binding induced line-broadening resulted in a fewer number of intermolecular NOE-derived distance constraints used for the ischemin-bound structure determination than that for MS456, i.e. 25 versus 53, respectively. Nevertheless, the ischemin/CBP BRD structure is better defined than the latter, consistent with its higher affinity. Ischemin binds across the entrance of the acetyl-lysine binding pocket in an extended conformation with its phenoxyl group forming a hydrogen bond (˜2.8 Å) to the amide nitrogen of Asn1168 in CBP. The latter is a highly conserved residue in the BRDs whose amide nitrogen is hydrogen-bonded to the acetyl oxygen of the acetyl-lysine in a biological binding partner as seen with acetylated-lysine 20 of histone H4 recognition by the CBP BRD (FIG. 1B vs. 1C). The sulfonate group forms electrostatic interactions with quanidinium group of Arg1173 in the BC loop and possibly also with side chain amide of G1n1113 in the ZA loop.

Ischemin in the acetyl-lysine binding pocket is sandwiched through hydrophobic and aromatic interactions between the diazobenzene and Leu1109, Prol 110 and Val 174 on one side, Leu1120 and I1e1122 in the ZA loop on the other. Since all the diazobenzenes contain a para-phenoxyl group, a hydrogen bond between the phenoxyl with Asn1168 is likely present in all the compounds when bound to the CBP BRD. As such, this structure explains the SAR data presented in Table 3. For instance, with a para-sulfonate in the diazonbenzene, ortho- but not meta-substitution of methyl groups on the phenol ring results in a marked increase in the lead's ability to inhibit p53-dependent p21 luciferase activity, e.g. MS450, MS451, and MS101 versus MS453 and MS110. Ortho-substitution of a larger alkyl group such as ethyl (MS113), propyl (MS123), isopropyl (MS105), or t-butyl (MS111) showed reduced activity on p21 inhibition as compare to that of ortho-methyl. The small hydrophobic group at ortho-position is due to its possible interaction with a small hydrophobic cavity formed with I1e1122, Tyr1125 and Tyr1167 that is positioned next to the conserved Asn1168 in the acetyl-lysine binding pocket.

When resided at meta-position in diazobenzene, sulfonate establishes electrostatic interactions with quanidinium side chain of Arg1173; this alters CBP preference for substitutions on the aromatic ring. For instance, inhibition of p21 expression seems less sensitive to variations of size and position of hydrophobic substituent groups on the phenol. Nevertheless, ortho-propyl (MS126) and ortho-ethyl-keto (MS127) substituted diazobenzenes exhibit 93.5% and 86.8% inhibition activity, respectively. This preferred ortho-substituent likely interacts with side chains of I1e1122, Tyr1125 and Tyr1167, a small hydrophobic pocket embedded in the acetyl-lysine binding site. With a meta-amino substituent, which electron-donating functionality may aid formation of a hydrogen bond between the phenoxyl in the diazobenzene and side chain amide of Asn1168 of the protein, ischemin nearly completely suppresses the p21 expression.

Monitoring change of intrinsic tryptophan fluorescence of a protein induced by ligand binding can be used to determine ligand binding affinity (K_{D}). This assay was used to assess ligand binding to the CBP BRD and ischemin binding to the BRDs from other transcription proteins as follows. The chemical ligands were prepared at 500-850 μM in the PBS buffer. Their serial dilutions by a factor of 1.5 in a 384-wells black plate were carried out using a Tecan EVO200 liquid handler down to a concentration of 0.5 nM. Protein was added to the compounds to a final concentration in each well of 5 μM. Tryptophan fluorescence of the protein was measured (with excitation set at 280 nm, emission at 350 nm) on a Tecan Safire2 reader. Inner filter correction was introduced to take into account the possible intrinsic fluorescence of the compound. The results were plotted using the equation: (Fo-F)/Fo=Bmax*[ligand free]/(K_{D}+[ligand free]), where Fo is fluorescence of the free protein, (Fo-F)/Fo, Fraction bound, Bmax, ideally equal to 1 (reaches saturation). K_{D} was calculated based on the curve fitting.

While many ischemin binding residues in the acetyl-lysine binding pocket are conserved among human BRDs, it was observed that ischemin exhibits up to five-fold selectivity for the CBP BRD over several other human BRDs including PCAF, BRD41, BAZ1B and BAZ2B as determined by an in vitro tryptophan fluorescence binding assay described above. The level of selectivity may attribute to several ischemin binding residues in CBP such as Pro1110, G1n1113 and Arg1173 that are not conserved in other human BRDs. Collectively, the new structure provides the detailed molecular basis of ischemin recognition by the CBP BRD.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A compound of formula (1): 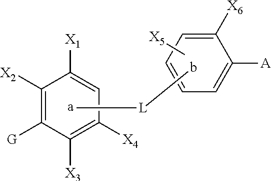
or a pharmaceutically acceptable salt form thereof, wherein:
A is selected from the group consisting of: 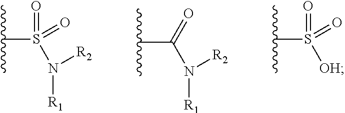
L is a linking group selected from: 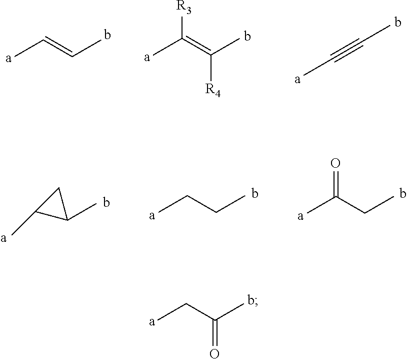
G is a heteroatom containing group capable of accepting a hydrogen bond or donating a hydrogen bond, or G is fused to X₂ or X₃ to form a heterocyclic ring system capable of accepting or donating a hydrogen bond;
X₁ and X₄ are independently selected from the group consisting of: H, C₁₋₁₀ alkyl, C₁₋₁₀ perfluoroalkyl, halogen, nitrile, hydroxy, C₁₋₁₀ alkoxy, C₁₋₁₀ perfluoroalkoxy, C₁₋₁₀ thioalkyl, C₁₋₁₀ perfluoroalkyl, amine, alkylamino, C₁₋₁₀ acylamino, aryl, heteroaryl, carboxamido, carboxyl, and carboalkoxy;
X₂ and X₃ are independently selected from the group consisting of: H, C₁₋₁₀ alkyl, C₁₋₁₀ perfluoroalkyl, halogen, nitrile, hydroxy, C₁₋₁₀ alkoxy, C₁₋₁₀ perfluoroalkoxy, C₁₋₁₀ thioalkyl, C₁₋₁₀ perfluoroalkyl, amine, alkylamino, C₁₋₁₀ acylamino, aryl, heteroaryl, carboxamide, and C₂₋₁₀ acyl;
optionally, X₁ and X₂ may come together to form a cycloalkyl, heterocycloalkyl, aromatic or heteroaromatic ring system;
X₅ and X₆ are independently selected from the group consisting of: H, C₁₋₁₀ alkyl, C₁₋₁₀ alkoxy, C₁₋₁₀ perfluoroalkyl, halogen, and nitrile;
R₁ is selected from the group consisting of: substituted or unsubstituted aryl, substituted or unsubstituted heteroaryl, and substituted or unsubstituted C₁₋₁₀ alkyl;
R₂ is selected from the group consisting of: H and C₁₋₁₀ alkyl;
optionally, R₁ and R₂ may come together to form a substituted or unsubstituted heterocycloalkyl ring system; and
R₃ and R₄ are independently selected from the group consisting of: H and C₁₋₁₀ alkyl.

2. The compound of claim 1, wherein A is:

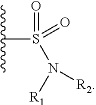

3. The compound of claim 1, wherein L is selected from the group consisting of:

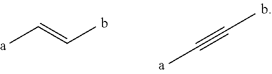

4-27. (canceled)

28. The compound of claim 1, wherein the compound is a compound of formula (1A): 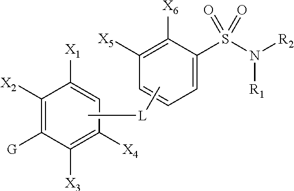
or a pharmaceutically acceptable salt form thereof, wherein:
L is selected from the group consisting of: 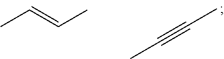
G is selected from the group consisting of: OH, CH₂OH, NH₂, SH, C(O)H, CO₂H, OC(O)HCN, NHC(O)H, NH(SO₂)H, NHC(O)NH₂, NHCN, CH(CN)₂, F, Cl, OSO₃H, ONO₂H, and NO₂, or G is fused to X₂ to form a heterocyclic ring system capable of accepting or donating a hydrogen bond;
X₁ is a protected or unprotected amine;
X₂ and X₃ are independently selected from the group consisting of: H, C₁₋₁₀ alkyl, halogen;
X₄, X₅, and X₆ are H;
R₁ is selected the group consisting of: substituted C₁₋₁₀ alkyl, aryl, and heteroaryl;
R₂ is H.

29-37. (canceled)

38. The compound of claim 1, wherein the compound is selected from the group consisting of:

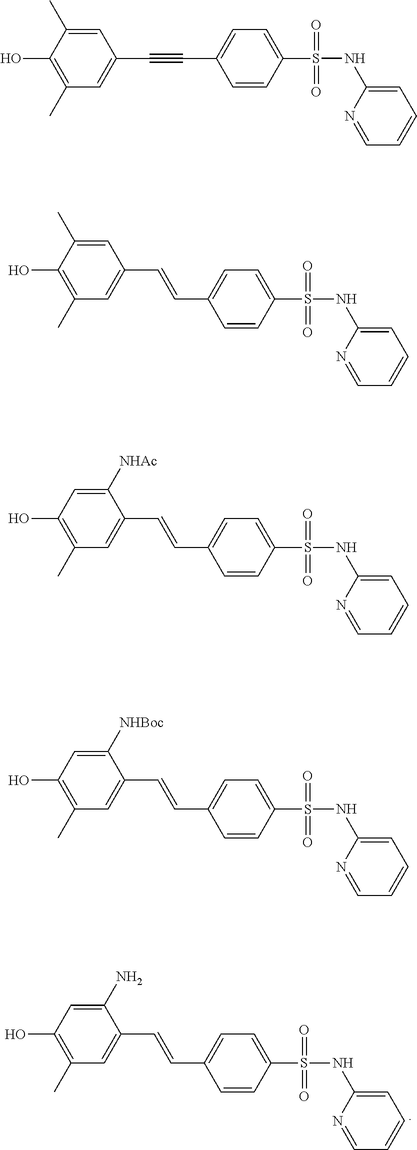 [SMILES: Cc1cc(C#Cc2ccc(S(=O)(=O)Nc3ccccn3)cc2)cc(C)c1O ; Cc1cc(/C=C/c2ccc(S(=O)(=O)Nc3ccccn3)cc2)cc(C)c1O ; CC(=O)Nc1cc(O)c(C)cc1/C=C/c1ccc(S(=O)(=O)Nc2ccccn2)cc1 ; Cc1cc(/C=C/c2ccc(S(=O)(=O)Nc3ccccn3)cc2)c(NC(=O)OC(C)(C)C)cc1O ; Cc1cc(/C=C/c2ccc(S(=O)(=O)Nc3ccccn3)cc2)c(N)cc1O]

39. A compound of formula (2): 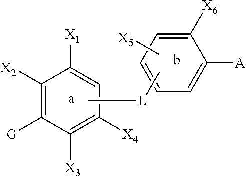
or a pharmaceutically acceptable salt form thereof, wherein:
A is selected from the group consisting of: 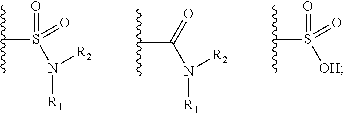
L is: 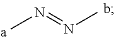
G is a heteroatom containing group capable of accepting a hydrogen bond or donating a hydrogen bond, or G is fused to X₂ or X₃ to form a heterocyclic ring system capable of accepting or donating a hydrogen bond;
X₁ and X₄ are independently selected from the group consisting of: H, C₁₋₁₀ alkyl, C₁₋₁₀ perfluoroalkyl, halogen, nitrile, hydroxy, C₁₋₁₀ alkoxy, C₁₋₁₀ perfluoroalkoxy, C₁₋₁₀ thioalkyl, C₁₋₁₀ perfluoroalkyl, amine, alkylamino, C₁₋₁₀ acylamino, aryl, heteroaryl, carboxamido, carboxyl, and carboalkoxy;
X₂ and X₃ are independently selected from the group consisting of: H, C₁₋₁₀ alkyl, C₁₋₁₀ perfluoroalkyl, halogen, nitrile, hydroxy, C₁₋₁₀ alkoxy, C₁₋₁₀ perfluoroalkoxy, C₁₋₁₀ thioalkyl, C₁₋₁₀ perfluoroalkyl, amine, alkylamino, C₁₋₁₀ acylamino, aryl, heteroaryl, carboxamide, and C₂₋₁₀ acyl;
optionally, X₁ and X₂ may come together to form a cycloalkyl, heterocycloalkyl, aromatic or heteroaromatic ring system;
X₅ and X₆ are independently selected from the group consisting of: H, C₁₋₁₀ alkyl, C₁₋₁₀ alkoxy, C₁₋₁₀ perfluoroalkyl, halogen, and nitrile;
R₁ is selected from the group consisting of: substituted or unsubstituted aryl, substituted or unsubstituted heteroaryl, and substituted or unsubstituted C₁₋₁₀ alkyl;
R₂ is selected from the group consisting of: H and C₁₋₁₀ alkyl;
optionally, R₁ and R₂ may come together to form a substituted or unsubstituted heterocycloalkyl ring system; and
R₃ and R₄ are independently selected from the group consisting of: H and C₁₋₁₀ alkyl.

40. The compound of claim 39, wherein A is:

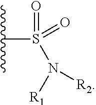

41. The compound of claim 39, wherein G is fused to X₂ or X₃ to form a heterocyclic ring system capable of accepting or donating a hydrogen bond.

42-57. (canceled)

58. The compound of claim 39, wherein the compound is a compound of formula (2A): 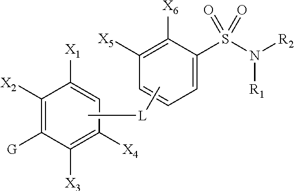
or a pharmaceutically acceptable salt form thereof, wherein:
L is: 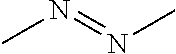
G is selected from the group consisting of: OH, CH₂OH, NH₂, SH, C(O)H, CO₂H, OC(O)HCN, NHC(O)H, NH(SO₂)H, NHC(O)NH₂, NHCN, CH(CN)₂, F, Cl, OSO₃H, ONO₂H, and NO₂;
X₁ is H or a protected or unprotected amine;
X₂ and X₃ are independently selected from the group consisting of: H, halogen, hydroxyl, C₁₋₁₀ alkyl, C₁₋₁₀ perfluoroalkyl, and C₁₋₁₀ alkoxy;
X₄ is H;
X₅ and X₆ are independently selected from the group consisting of: H, halogen, hydroxyl, C₁₋₁₀ alkyl, and C₁₋₁₀ alkoxy;
R₁ is selected the group consisting of: substituted C₁₋₁₀ alkyl, aryl, and heteroaryl; and
R₂ is H.

59-64. (canceled)

65. The compound of claim 39, wherein the compound is a compound of formula (2B): 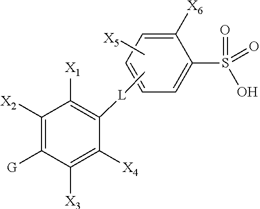
or a pharmaceutically acceptable salt form thereof, wherein:
L is: 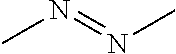
G is OH;
X₁ and X₄ are H;
X₂ and X₃ are independently selected from the group consisting of: H, halogen, hydroxyl, C₁₋₁₀ alkyl, C₁₋₁₀ perfluoroalkyl, and C₁₋₁₀ alkoxy; and
X₅ and X₆ are independently selected from the group consisting of: H, halogen, hydroxyl, C₁₋₁₀ alkyl, and C₁₋₁₀ alkoxy.

66. The compound of claim 39, wherein the compound is selected from the group consisting of:

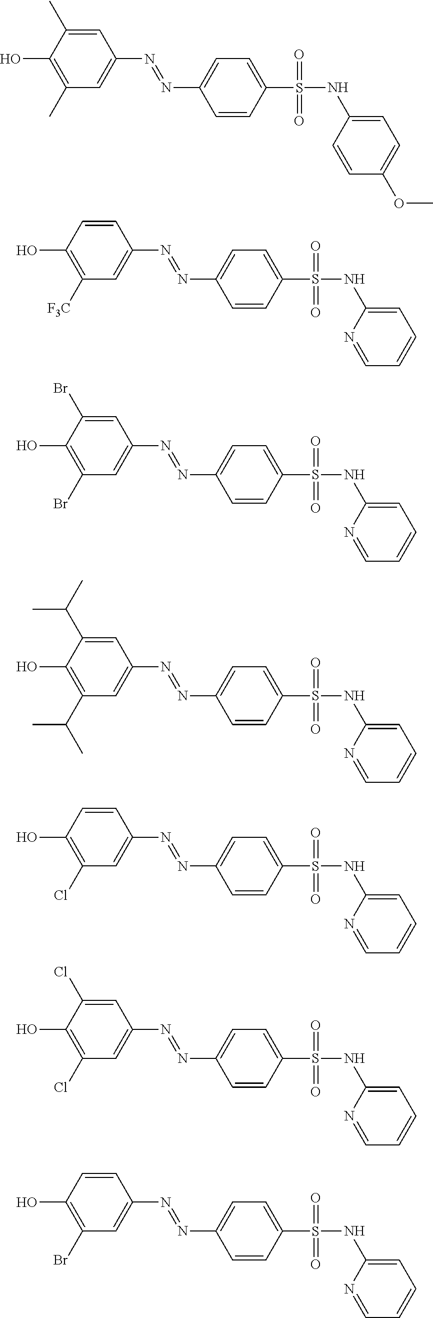

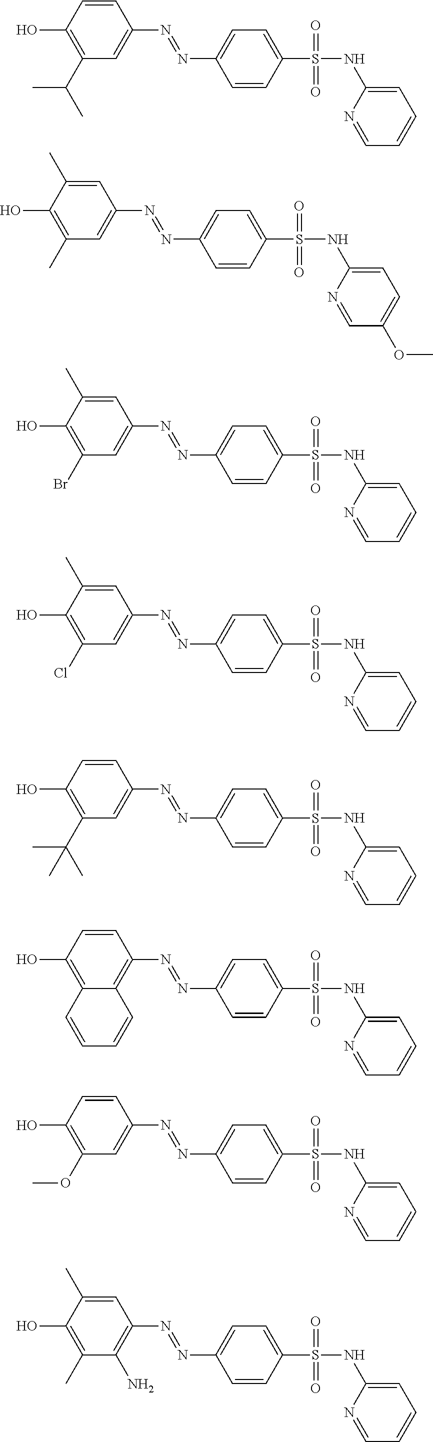

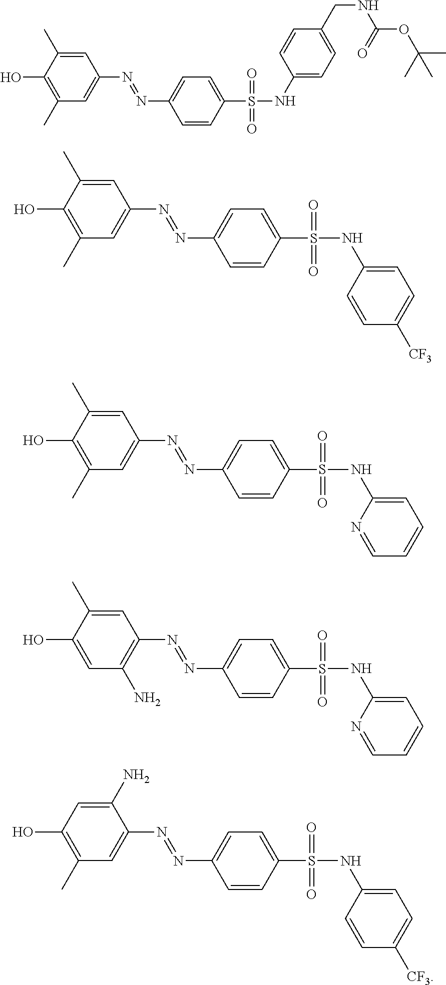

67. (canceled)

68. A method of treating cancer in a patient, the method comprising administering a therapeutically effective amount of a compound of claim 1, or a pharmaceutically acceptable salt form thereof, to the patient.

69. (canceled)

70. The method of claim 68, wherein the method further comprises administering a therapeutically effective amount of an anticancer agent to the patient.

71-78. (canceled)

79. A method for treating HIV/AIDS in a patient, the method comprising administering a therapeutically effective amount of a compound of claim 1, or a pharmaceutically acceptable salt form thereof, to the patient.

80-88. (canceled)

89. A method of treating an inflammatory disease or autoimmune disease in a patient, the method comprising administering a therapeutically effective amount of a compound of claim 1, or a pharmaceutically acceptable salt form thereof, to the patient.

90-92. (canceled)

93. A method of treating a neurological disorder in a patient where NF-kB is implicated in the pathology of the disorder, the method comprising administering a therapeutically effective amount of a compound of claim 1, or a pharmaceutically acceptable salt form thereof, to the patient.

94-98. (canceled)

99. A method for treating a retroviral infection in a patient, the method comprising administering a therapeutically effective amount of a compound of claim 1, or a pharmaceutically acceptable salt form thereof, to the patient.

100. A method for treating myocardial hypertrophy in a patient, the method comprising administering a therapeutically effective amount of a compound of claim 1, or a pharmaceutically acceptable salt form thereof, to the patient.

101-129. (canceled)

130. A method of treating cancer in a patient, the method comprising administering a therapeutically effective amount of a compound of claim 39, or a pharmaceutically acceptable salt form thereof, to the patient.

131. A method for treating HIV/AIDS in a patient, the method comprising administering a therapeutically effective amount of a compound of claim 39, or a pharmaceutically acceptable salt form thereof, to the patient.

132. A method of treating an inflammatory disease or autoimmune disease in a patient, the method comprising administering a therapeutically effective amount of a compound of claim 39, or a pharmaceutically acceptable salt form thereof, to the patient.

133. A method of treating a neurological disorder in a patient where NF-kB is implicated in the pathology of the disorder, the method comprising administering a therapeutically effective amount of a compound of claim 39, or a pharmaceutically acceptable salt form thereof, to the patient.

134. A method for treating a retroviral infection in a patient, the method comprising administering a therapeutically effective amount of a compound of claim 39, or a pharmaceutically acceptable salt form thereof, to the patient.

135. A method for treating myocardial hypertrophy in a patient, the method comprising administering a therapeutically effective amount of a compound of claim 39, or a pharmaceutically acceptable salt form thereof, to the patient.
